SEQUENCE LISTING (3 sequences)

SEQ ID NO: 1 - protein, 373 aa, Homo sapiens [sequence omitted]

SEQ ID NO: 2 - protein, 373 aa, Homo sapiens
MTGWSCLVTGAGGFLGQRIIRLLVKEKELKEIRVLDKAFGPELREEFSKLQNKTKLTVLEGDILDEPFLKRACQDVSVIIHTACIIDVFGVTHRESIMNVNVKGTQLLLEACVQASVPVFIYTSSIEVAGPNSYKEIIQNGHEEEPLENTWPAPYPHSKKLAEKAVLAANGWNLKNGGTLYTCALRPMYIYGEGSRFLSASINEALNNNGILSSVGKFSTVNPVYVGNVAWAHILALRALQDPKKAPSIRGQFYYISDDTPHQSYDNLNYTLSKEFGLRLDSRWSFPLSLMYWIGFLLEIVSFLLRPIYTYRPPFNRHIVTLSNSVFTFSYKKAQRDLAYKPLYSWEEAKQKTVEWVGSLVDRHKETLKSKTQ

SEQ ID NO: 3 - protein, 386 aa, synthetic construct
MHHHHHHENLYFQGTGWSCLVTGAGGFLGQRIIRLLVKEKELKEIRVLDKAFGPELREEFSKLQNKTKLTVLEGDILDEPFLKRACQDVSVIIHTACIIDVFGVTHRESIMNVNVKGTQLLLEACVQASVPVFIYTSSIEVAGPNSYKEIIQNGHEEEPLENTWPAPYPHSKKLAEKAVLAANGWNLKNGGTLYTCALRPMYIYGEGSRFLSASINEALNNNGILSSVGKFSTVNPVYVGNVAWAHILALRALQDPKKAPSIRGQFYYISDDTPHQSYDNLNYTLSKEFGLRLDSRWSFPLSLMYWIGFLLEIVSFLLRPIYTYRPPFNRHIVTLSNSVFTFSYKKAQRDLAYKPLYSWEEAKQKTVEWVGSLVDRHKETLKSKTQ

(19) United States
(12) Patent Application Publication
(10) Pub. No.: US 2025/0241905 A1
(43) Pub. Date: Jul. 31, 2025

(54) 3-BETA-HSD1 INHIBITORS AND COMPOSITIONS AND USES THEREOF

(71) Applicant: The Cleveland Clinic Foundation, Cleveland, OH (US)

(72) Inventors: Nima Sharifi, Cleveland, OH (US); Shaun R. Stauffer, Cleveland, OH (US); Joseph Alvarado, Cleveland, OH (US); Christopher M. Goins, Cleveland, OH (US); Dhiraj P. Sonawane, Cleveland, OH (US); Steven R. Martinez, Cleveland, OH (US); Sang Hoon Han, Cleveland, OH (US)

(21) Appl. No.: 18/878,161

(22) PCT Filed: Jun. 23, 2023
(86) PCT No.: PCT/US2023/068982

§ 371 Date: Dec. 23, 2024

Related U.S. Application Data
(60) Provisional application No. 63/354,988, filed on Jun. 23, 2022.

(51) Int. Cl. A61K 31/4709; A61K 31/4355; A61K 31/436; A61K 31/4375; A61K 31/47; A61K 31/4747; A61K 31/498; A61K 31/517; A61P 35/00; C07D 215/14; C07D 221/04; C07D 239/74; C07D 241/42; C07D 401/04; C07D 401/06; C07D 405/12; C07D 413/04; C07D 471/04; C07D 491/052; C07D 491/107; C07D 491/113

(52) U.S. Cl. CPC A61K 31/4709; A61K 31/4355; A61K 31/436; A61K 31/4375; A61K 31/47; A61K 31/4747; A61K 31/498; A61K 31/517; A61P 35/00; C07D 215/14; C07D 221/04; C07D 239/74; C07D 241/42; C07D 401/04; C07D 401/06; C07D 405/12; C07D 413/04; C07D 471/04; C07D 491/052; C07D 491/107; C07D 491/113

(57) ABSTRACT

Disclosed herein are compounds that inhibit the function of 3β-hydroxysteroid dehydrogenase (3βHSD1), pharmaceutical compositions comprising the compounds, and methods of using the compounds, e.g., for the treatment of prostate cancer, breast cancer, and other diseases dependent on the activity of 3βHSD1.

### CROSS-REFERENCE TO RELATED APPLICATIONS

This application claims priority to and the benefit of U.S. Provisional Patent Application No. 63/354,988, filed on Jun. 23, 2022, which is incorporated herein by reference in its entirety.

### STATEMENT REGARDING FEDERALLY SPONSORED RESEARCH

This invention was made with government support under CA261995, CA236780, CA172382 and CA249279 awarded by the National Institutes of Health, and W81XWH-20-1-0137 awarded by the Department of Defense. The government has certain rights in the invention.

### SEQUENCE LISTING STATEMENT

The contents of the electronic sequence listing titled CCF-40846.601.xml (Size: 4,726 bytes; and Date of Creation: Jun. 23, 2023) is herein incorporated by reference in its entirety.

### TECHNICAL FIELD

The present disclosure relates generally to compounds that inhibit the function of 3β-hydroxysteroid dehydrogenase (3βHSD1), pharmaceutical compositions comprising the compounds, and methods of using the compounds, e.g., for the treatment of prostate cancer, breast cancer, endometrial cancer, and other diseases dependent on the activity of 3βHSD1.

### BACKGROUND

The long-standing frontline and most effective treatment for advanced prostate cancer had been androgen deprivation therapy (ADT), with medical or surgical castration. Gonadal testosterone is the major physiologic source of androgens in men, and tumor responses occur in 80-90% of patients treated with ADT. In this context, median response durations vary widely, reflecting tumor heterogeneity. Prostate cancer eventually becomes resistant to ADT, often by way of mechanisms that allow tumor generation of testosterone and/or dihydrotestosterone (DHT) from extragonadal (mainly adrenal-derived) precursor steroids and other mechanisms that allow for re-activation of the androgen receptor (AR). Next-generation hormonal therapies have been developed to counter resistance mechanisms that drive the development of castration-resistant prostate cancer (CRPC). Specifically, abiraterone inhibits the enzyme 17-hydroxylase/17,20-lyase (CYP17A1) and blocks the synthesis of adrenal precursor steroids, and enzalutamide and apalutamide are potent competitive inhibitors of the AR. However, new therapies for treating prostate cancer, and related cancers, are needed.

### SUMMARY

Disclosed herein are compounds of formula (I):

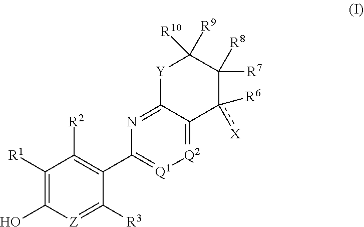

and pharmaceutically acceptable salts thereof, wherein:
R¹ is halo, cyano, or C₁-C₄ haloalkyl;
Z is selected from CR⁴ and N;
R², R³, and R⁴ are independently selected from hydrogen, halo, and C₁-C₄ alkyl;
Q₁ is CH or N;
Q₂ is CR⁵ or N;
R⁵ is selected from hydrogen, C₁-C₄ alkyl, halo, and C₁-C₄ haloalkyl;
--- represents the presence or absence of a bond; wherein, when --- represents the presence of a bond, X is oxo and R⁶ is absent; and wherein, when --- represents the absence of a bond, X is selected from —OR^{a} and —NR^{b}R^{c}, and R⁶ is selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, and aryl-C₁-C₄-alkyl;
Y is selected from —CH₂—, —NR^{d}—, —O—, —S—, —CH₂CH₂—, —NHCH₂—, —OCH₂—, —SCH₂—, and a bond; and
R⁷, R⁸, R⁹, and R¹⁰ are each independently selected from hydrogen, C₁-C₆ alkyl, C₂-C₆ alkenyl, C₂-C₆ alkynyl, C₁-C₆ alkoxy, hydroxy, cyano, halo, C₃-C₆ cycloalkyl, monocyclic 3- to 6-membered heterocyclyl having one heteroatom selected from O and N, aryl, heteroaryl, C₁-C₄-alkoxy-C₁-C₆-alkyl, hydroxy-C₁-C₆-alkyl, halo-C₁-C₆-alkyl, carboxy-C₁-C₆-alkyl, amino-C₁-C₆-alkyl, C₃-C₆-cycloalkyl-C₁-C₄-alkyl, aryl-C₁-C₄-alkyl, and heteroaryl-C₁-C₄-alkyl, wherein R⁷ and R⁸, together with the carbon atom to which they are attached, are optionally taken together to form a 3- to 6-membered ring; and
R^{a}, R^{b}, R^{c}, and R^{d} are each independently selected from hydrogen, C₁-C₄ alkyl, C₁-C₄-alkoxy-C₁-C₄-alkyl, heterocyclyl, and heterocyclyl-C₁-C₄-alkyl;
wherein each cycloalkyl, heterocyclyl, aryl, and heteroaryl is independently unsubstituted or substituted with 1, 2, or 3 substituents independently selected from C₁-C₆ alkyl, C₁-C₆ alkoxy, hydroxy, cyano, halo, and C₃-C₆ cycloalkyl.

In some embodiments, R¹ is fluoro, chloro, cyano, or trifluoromethyl. In some embodiments, R¹ is fluoro or cyano.

In some embodiments, R² and R³ are each independently selected from hydrogen, fluoro, and methyl. In some embodiments, R² and R³ are each independently selected from hydrogen and fluoro. In some embodiments, R² and R³ are each hydrogen.

In some embodiments, Z is CR⁴, and R⁴ is selected from hydrogen, halo, and methyl. In some embodiments, Z is CR⁴, and R⁴ is selected from hydrogen and halo. In some embodiments, Z is N.

In some embodiments, Q₁ is CH. In some embodiments, Q₁ is N.

In some embodiments, Q₂ is CR⁵, and R⁵ is selected from hydrogen and C₁-C₄-alkyl. In some embodiments, Q₂ is CR⁵, and R⁵ is hydrogen. In some embodiments, Q₂ is N.

In some embodiments, --- represents the presence of a bond, X is oxo, and R⁶ is absent. In some embodiments, --- represents the absence of a bond, X is selected from —OR^{a} and —NR^{b}R^{c}, and R⁶ is selected from hydrogen, C₁-C₈ alkyl, C₃-C₄ cycloalkyl, phenyl, a monocyclic 5- or 6-membered heteroaryl having 1 or 2 heteroatoms independently selected from N, O, and S, and phenyl-C₁-C₂-alkyl, wherein R^{a} is selected from hydrogen, C₁-C₃ alkyl, heterocyclyl, and heterocyclyl-C₁-C₄-alkyl, and R^{b} and R^{c} are each hydrogen, wherein the heterocyclyl is a monocyclic 4- to 6-membered heterocyclyl having one heteroatom selected from O and N. In some embodiments, --- represents the absence of a bond; X is selected from —OH and —NH₂; and R⁶ is selected from hydrogen, C₁-C₃ alkyl, C₃-C₄ cycloalkyl, phenyl, pyridyl, oxazolyl, and phenyl-C₁-C₂-alkyl.

In some embodiments, Y is selected from —CH₂—, —NR^{d}—, —O—, —CH₂CH₂—, and a bond, wherein R^{d} is selected from hydrogen, C₁-C₄ alkyl, and C₁-C₄-alkoxy-C₁-C₄-alkyl. In some embodiments, Y is selected from —CH₂—, —NH—, —CH₂CH₂—, and a bond.

In some embodiments, wherein R⁷ and R⁸ are each independently selected from hydrogen, C₁-C₃ alkyl, hydroxy, cyano, halo, C₃-C₆ cycloalkyl, a monocyclic 3- to 6-membered heterocyclyl having one heteroatom selected from O and N, aryl, C₁-C₂-alkoxy-C₁-C₃-alkyl, hydroxy-C₁-C₃-alkyl, halo-C₁-C₃-alkyl, carboxy-C₁-C₃-alkyl, amino-C₁-C₃-alkyl, C₃-C₄-cycloalkyl-C₁-C₂-alkyl, aryl-C₁-C₃-alkyl, and heteroaryl-C₁-C₂-alkyl, wherein the cycloalkyl and the heterocyclyl are each independently unsubstituted or substituted with one substituent selected from hydroxy, methyl, halo, and cyano. In some embodiments, R⁷ and R⁸ are each independently selected from hydrogen, C₁-C₃ alkyl, hydroxy, cyano, halo, C₃-C₆ cycloalkyl, aryl, halo-C₁-C₃-alkyl, carboxy-C₁-C₃-alkyl, C₃-C₄-cycloalkyl-C₁-C₃-alkyl, aryl-C₁-C₂-alkyl, and heteroaryl-C₁-C₃-alkyl. In some embodiments, R⁷ and R⁸, together with the carbon atom to which they are attached, are taken together to form a 3- to 6-membered cycloalkyl or a 3- to 6-membered monocyclic heterocyclyl having one or two oxygen atoms. In some embodiments, R⁷ and R⁸, together with the carbon atom to which they are attached, are taken together to form a 3- or 4-membered cycloalkyl.

In some embodiments, R⁹ and R¹⁰ are each independently hydrogen or C₁-C₄ alkyl. In some embodiments, R⁹ and R¹⁰ are each independently hydrogen or methyl.

In some embodiments, the compound is selected from the group consisting of:

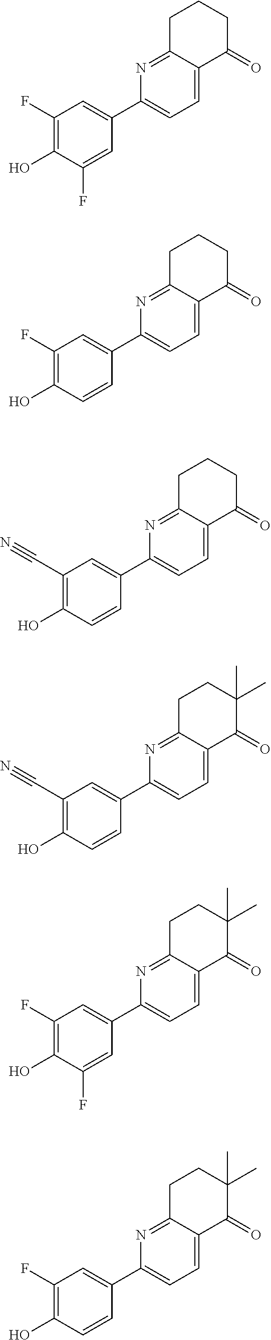 [SMILES: O=C1CCCc2nc(-c3cc(F)c(O)c(F)c3)ccc21 ; O=C1CCCc2nc(-c3ccc(O)c(F)c3)ccc21 ; N#Cc1cc(-c2ccc3c(n2)CCCC3=O)ccc1O ; CC1(C)CCc2nc(-c3ccc(O)c(C#N)c3)ccc2C1=O ; CC1(C)CCc2nc(-c3cc(F)c(O)c(F)c3)ccc2C1=O ; CC1(C)CCc2nc(-c3ccc(O)c(F)c3)ccc2C1=O]

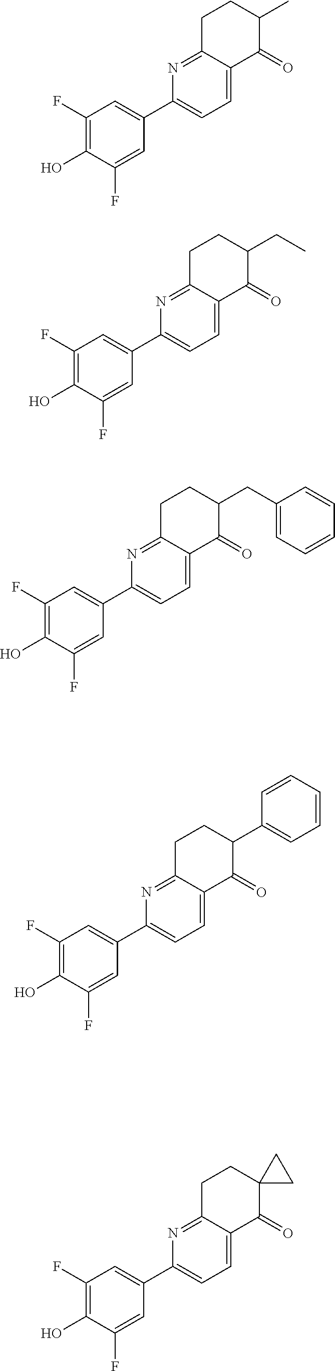 [SMILES: CC1CCc2nc(-c3cc(F)c(O)c(F)c3)ccc2C1=O ; CCC1CCc2nc(-c3cc(F)c(O)c(F)c3)ccc2C1=O ; O=C1c2ccc(-c3cc(F)c(O)c(F)c3)nc2CCC1Cc1ccccc1 ; O=C1c2ccc(-c3cc(F)c(O)c(F)c3)nc2CCC1c1ccccc1 ; O=C1c2ccc(-c3cc(F)c(O)c(F)c3)nc2CCC12CC2]

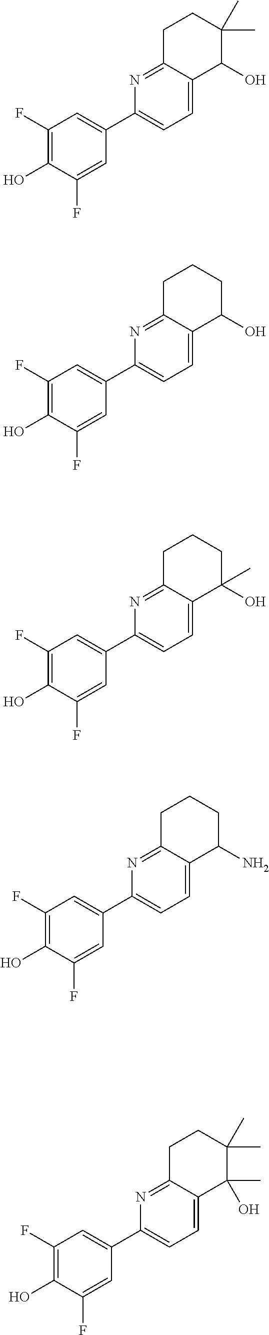 [SMILES: CC1(C)CCc2nc(-c3cc(F)c(O)c(F)c3)ccc2C1O ; Oc1c(F)cc(-c2ccc3c(n2)CCCC3O)cc1F ; CC1(O)CCCc2nc(-c3cc(F)c(O)c(F)c3)ccc21 ; NC1CCCc2nc(-c3cc(F)c(O)c(F)c3)ccc21 ; CC1(C)CCc2nc(-c3cc(F)c(O)c(F)c3)ccc2C1(C)O]

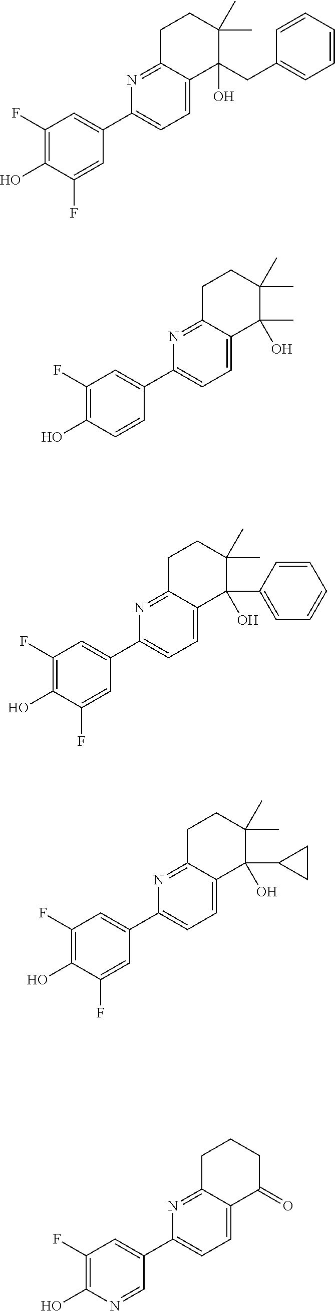 [SMILES: CC1(C)CCc2nc(-c3cc(F)c(O)c(F)c3)ccc2C1(O)Cc1ccccc1 ; CC1(C)CCc2nc(-c3ccc(O)c(F)c3)ccc2C1(C)O ; CC1(C)CCc2nc(-c3cc(F)c(O)c(F)c3)ccc2C1(O)c1ccccc1 ; CC1(C)CCc2nc(-c3cc(F)c(O)c(F)c3)ccc2C1(O)C1CC1 ; O=C1CCCc2nc(-c3cnc(O)c(F)c3)ccc21]

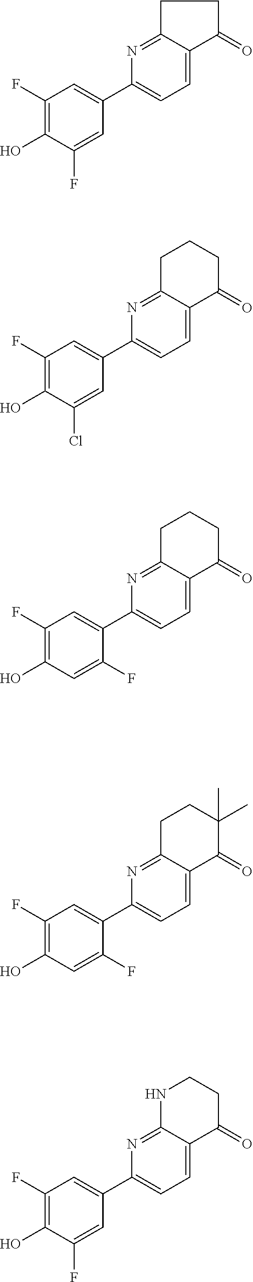 [SMILES: O=C1CCc2nc(-c3cc(F)c(O)c(F)c3)ccc21 ; O=C1CCCc2nc(-c3cc(F)c(O)c(Cl)c3)ccc21 ; O=C1CCCc2nc(-c3cc(F)c(O)cc3F)ccc21 ; CC1(C)CCc2nc(-c3cc(F)c(O)cc3F)ccc2C1=O ; O=C1CCNc2nc(-c3cc(F)c(O)c(F)c3)ccc21]

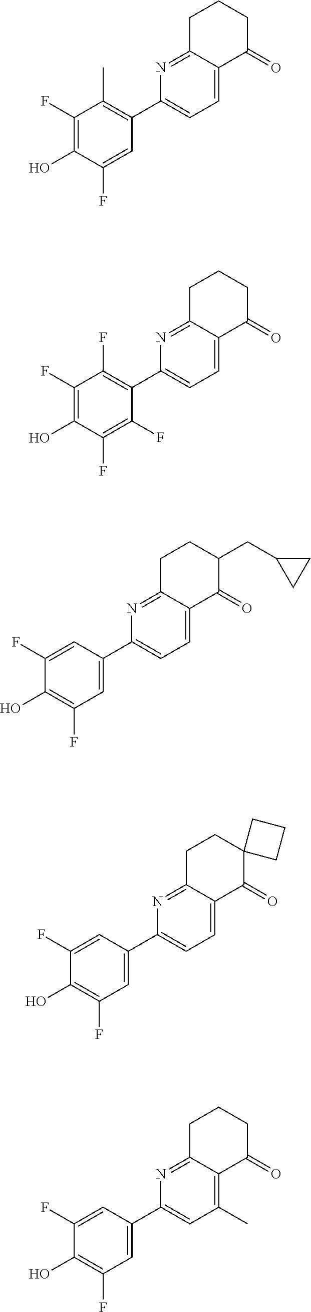 [SMILES: Cc1c(-c2ccc3c(n2)CCCC3=O)cc(F)c(O)c1F ; O=C1CCCc2nc(-c3c(F)c(F)c(O)c(F)c3F)ccc21 ; O=C1c2ccc(-c3cc(F)c(O)c(F)c3)nc2CCC1CC1CC1 ; O=C1c2ccc(-c3cc(F)c(O)c(F)c3)nc2CCC12CCC2 ; Cc1cc(-c2cc(F)c(O)c(F)c2)nc2c1C(=O)CCC2]

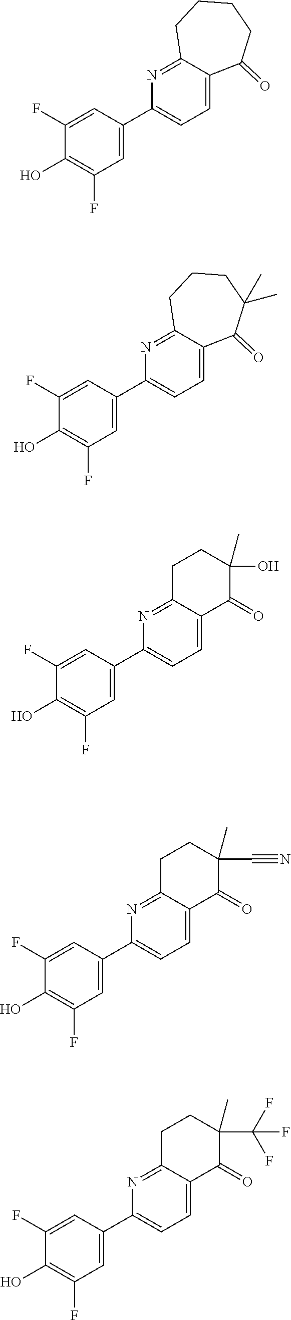 [SMILES: O=C1CCCCc2nc(-c3cc(F)c(O)c(F)c3)ccc21 ; CC1(C)CCCc2nc(-c3cc(F)c(O)c(F)c3)ccc2C1=O ; CC1(O)CCc2nc(-c3cc(F)c(O)c(F)c3)ccc2C1=O ; CC1(C#N)CCc2nc(-c3cc(F)c(O)c(F)c3)ccc2C1=O ; CC1(C(F)(F)F)CCc2nc(-c3cc(F)c(O)c(F)c3)ccc2C1=O]

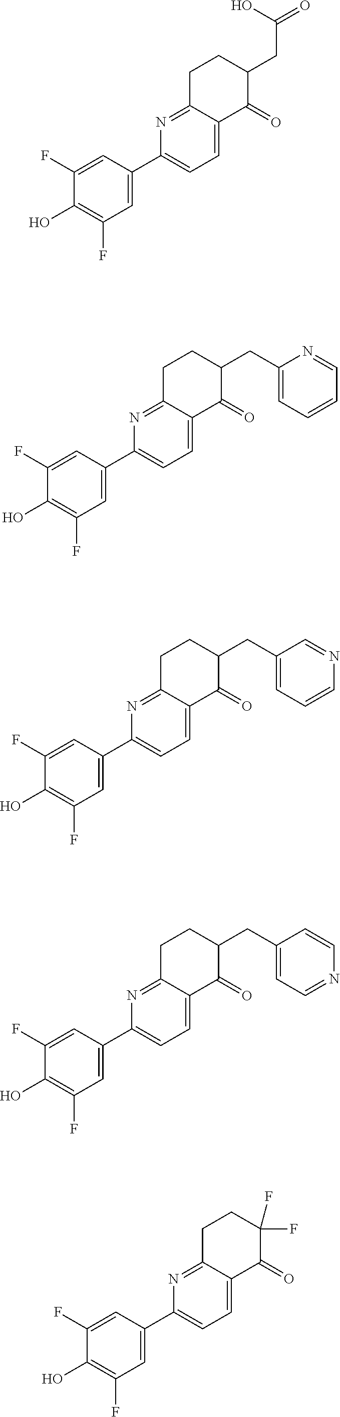 [SMILES: O=C(O)CC1CCc2nc(-c3cc(F)c(O)c(F)c3)ccc2C1=O ; O=C1c2ccc(-c3cc(F)c(O)c(F)c3)nc2CCC1Cc1ccccn1 ; O=C1c2ccc(-c3cc(F)c(O)c(F)c3)nc2CCC1Cc1cccnc1 ; O=C1c2ccc(-c3cc(F)c(O)c(F)c3)nc2CCC1Cc1ccncc1 ; O=C1c2ccc(-c3cc(F)c(O)c(F)c3)nc2CCC1(F)F]

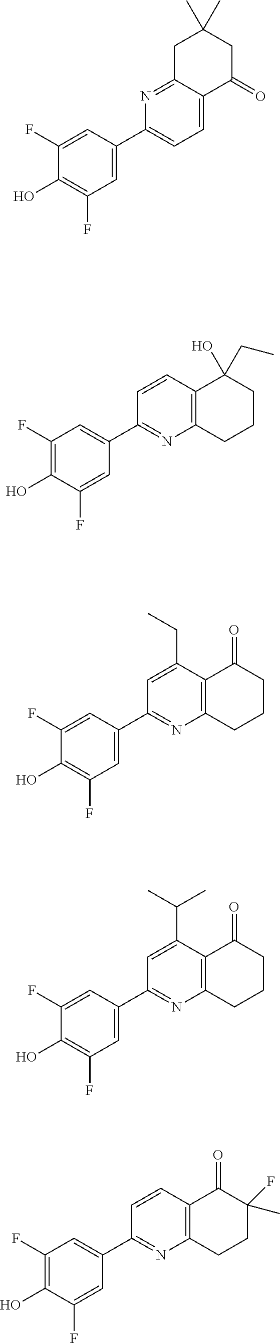 [SMILES: CC1(C)CC(=O)c2ccc(-c3cc(F)c(O)c(F)c3)nc2C1 ; CCC1(O)CCCc2nc(-c3cc(F)c(O)c(F)c3)ccc21 ; CCc1cc(-c2cc(F)c(O)c(F)c2)nc2c1C(=O)CCC2 ; CC(C)c1cc(-c2cc(F)c(O)c(F)c2)nc2c1C(=O)CCC2 ; CC1(F)CCc2nc(-c3cc(F)c(O)c(F)c3)ccc2C1=O]

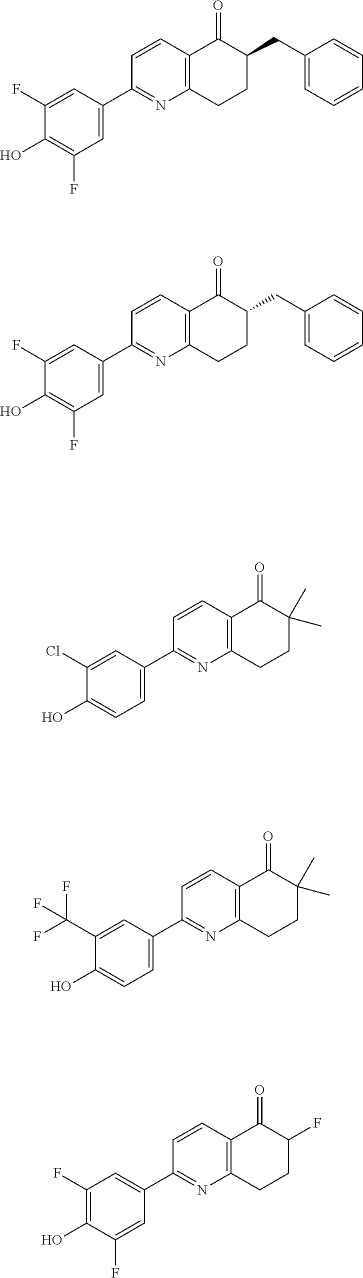 [SMILES: O=C1c2ccc(-c3cc(F)c(O)c(F)c3)nc2CC[C@@H]1Cc1ccccc1 ; O=C1c2ccc(-c3cc(F)c(O)c(F)c3)nc2CC[C@H]1Cc1ccccc1 ; CC1(C)CCc2nc(-c3ccc(O)c(Cl)c3)ccc2C1=O ; CC1(C)CCc2nc(-c3ccc(O)c(C(F)(F)F)c3)ccc2C1=O ; O=C1c2ccc(-c3cc(F)c(O)c(F)c3)nc2CCC1F]

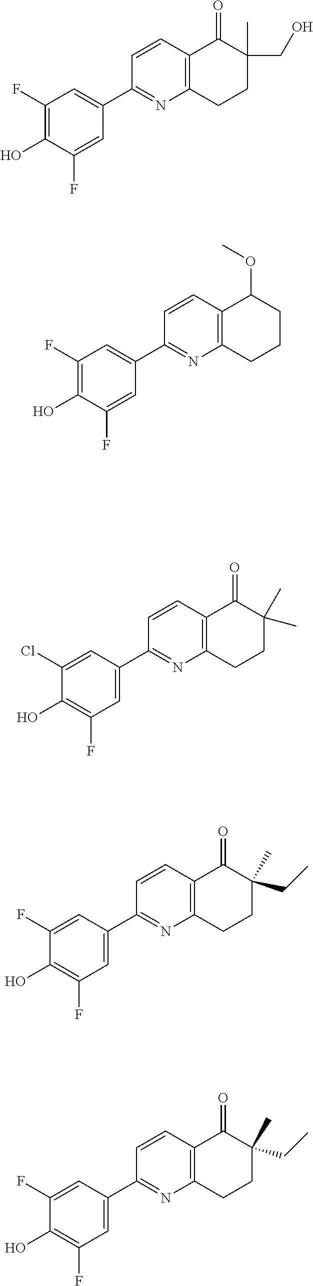 [SMILES: CC1(CO)CCc2nc(-c3cc(F)c(O)c(F)c3)ccc2C1=O ; COC1CCCc2nc(-c3cc(F)c(O)c(F)c3)ccc21 ; CC1(C)CCc2nc(-c3cc(F)c(O)c(Cl)c3)ccc2C1=O ; CC[C@@]1(C)CCc2nc(-c3cc(F)c(O)c(F)c3)ccc2C1=O ; CC[C@]1(C)CCc2nc(-c3cc(F)c(O)c(F)c3)ccc2C1=O]

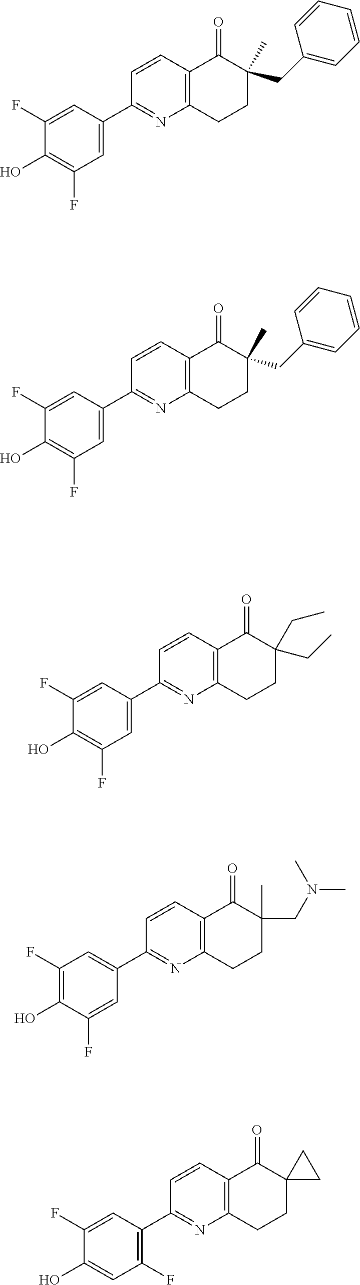 [SMILES: C[C@]1(Cc2ccccc2)CCc2nc(-c3cc(F)c(O)c(F)c3)ccc2C1=O ; C[C@@]1(Cc2ccccc2)CCc2nc(-c3cc(F)c(O)c(F)c3)ccc2C1=O ; CCC1(CC)CCc2nc(-c3cc(F)c(O)c(F)c3)ccc2C1=O ; CN(C)CC1(C)CCc2nc(-c3cc(F)c(O)c(F)c3)ccc2C1=O ; O=C1c2ccc(-c3cc(F)c(O)cc3F)nc2CCC12CC2]

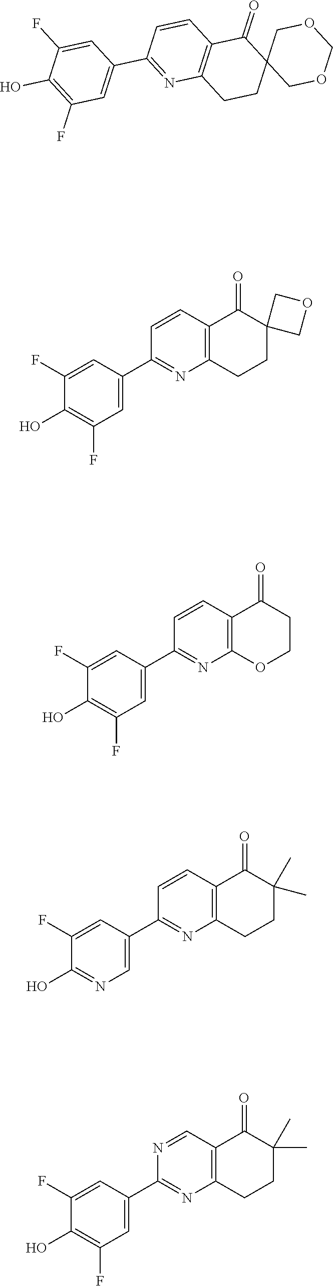 [SMILES: O=C1c2ccc(-c3cc(F)c(O)c(F)c3)nc2CCC12COCOC2 ; O=C1c2ccc(-c3cc(F)c(O)c(F)c3)nc2CCC12COC2 ; O=C1CCOc2nc(-c3cc(F)c(O)c(F)c3)ccc21 ; CC1(C)CCc2nc(-c3cnc(O)c(F)c3)ccc2C1=O ; CC1(C)CCc2nc(-c3cc(F)c(O)c(F)c3)ncc2C1=O]

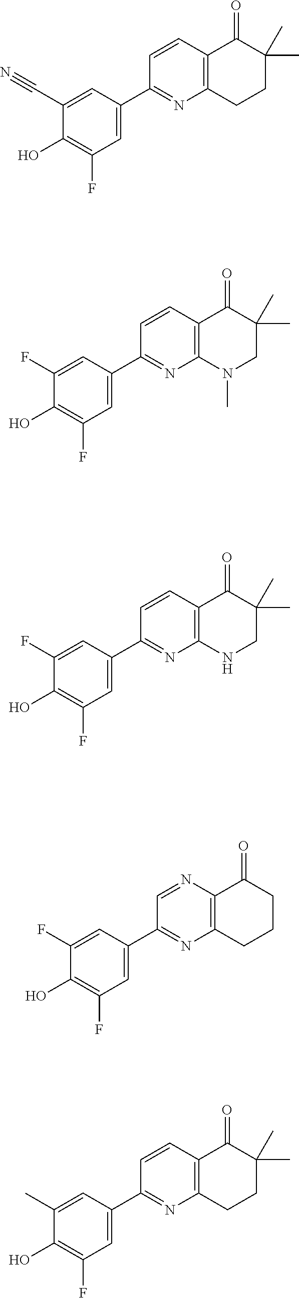

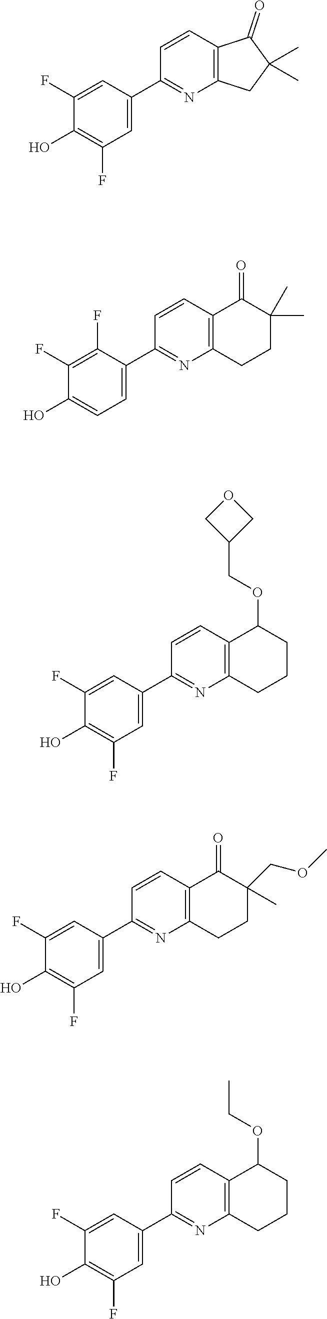 [SMILES: CC1(C)Cc2nc(-c3cc(F)c(O)c(F)c3)ccc2C1=O ; CC1(C)CCc2nc(-c3ccc(O)c(F)c3F)ccc2C1=O ; Oc1c(F)cc(-c2ccc3c(n2)CCCC3OCC2COC2)cc1F ; COCC1(C)CCc2nc(-c3cc(F)c(O)c(F)c3)ccc2C1=O ; CCOC1CCCc2nc(-c3cc(F)c(O)c(F)c3)ccc21]

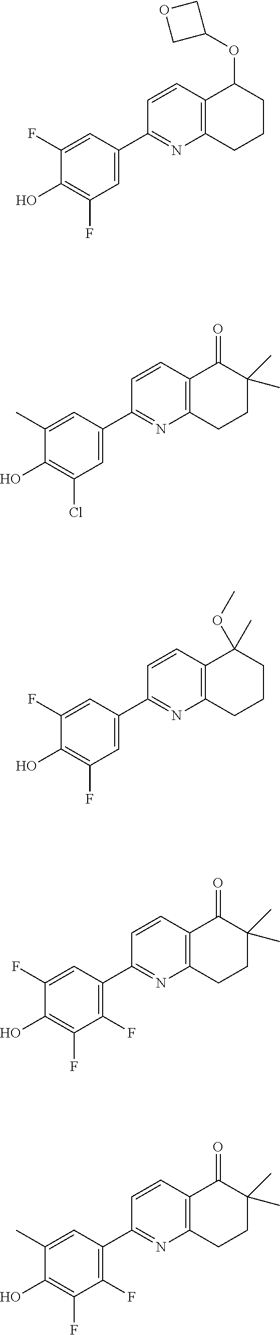

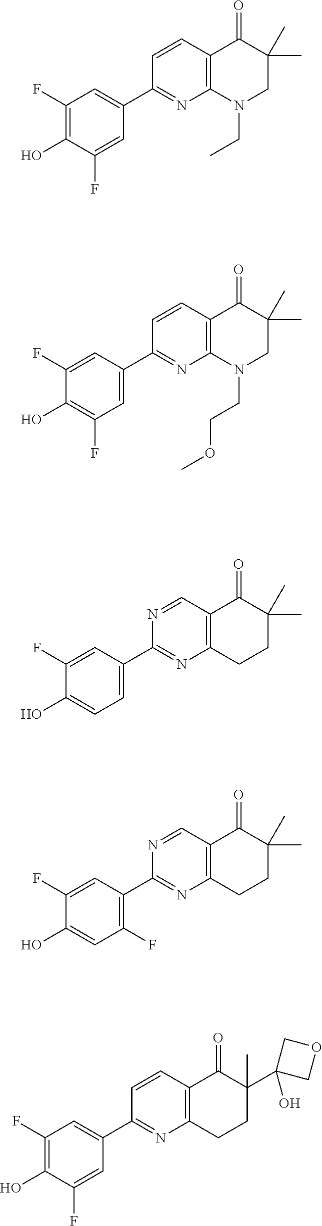 [SMILES: CCN1CC(C)(C)C(=O)c2ccc(-c3cc(F)c(O)c(F)c3)nc21 ; COCCN1CC(C)(C)C(=O)c2ccc(-c3cc(F)c(O)c(F)c3)nc21 ; CC1(C)CCc2nc(-c3ccc(O)c(F)c3)ncc2C1=O ; CC1(C)CCc2nc(-c3cc(F)c(O)cc3F)ncc2C1=O ; CC1(C2(O)COC2)CCc2nc(-c3cc(F)c(O)c(F)c3)ccc2C1=O]

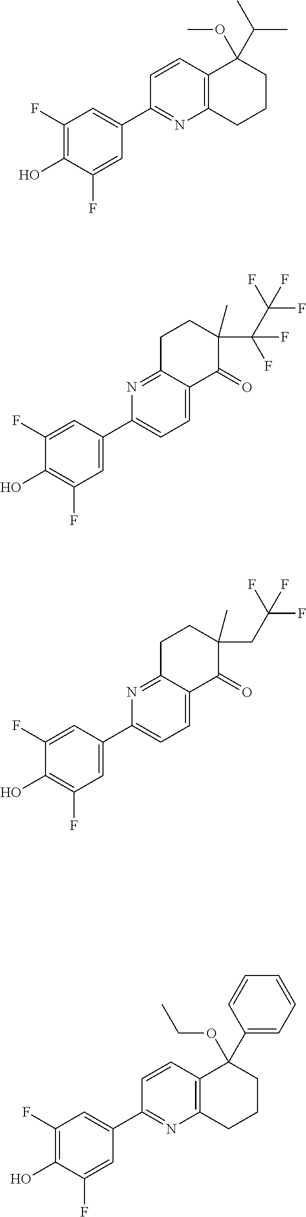 [SMILES: COC1(C(C)C)CCCc2nc(-c3cc(F)c(O)c(F)c3)ccc21 ; CC1(C(F)(F)C(F)(F)F)CCc2nc(-c3cc(F)c(O)c(F)c3)ccc2C1=O ; CC1(CC(F)(F)F)CCc2nc(-c3cc(F)c(O)c(F)c3)ccc2C1=O ; CCOC1(c2ccccc2)CCCc2nc(-c3cc(F)c(O)c(F)c3)ccc21]

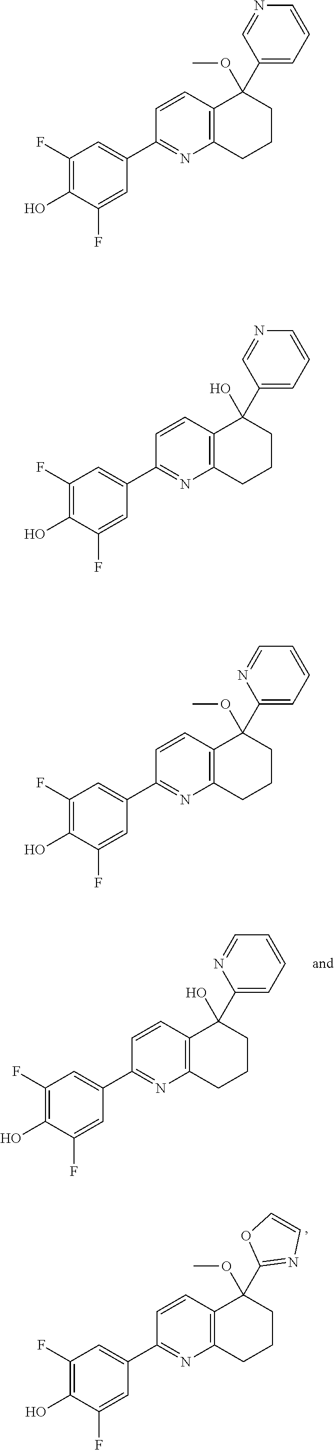 [SMILES: COC1(c2cccnc2)CCCc2nc(-c3cc(F)c(O)c(F)c3)ccc21 ; Oc1c(F)cc(-c2ccc3c(n2)CCCC3(O)c2cccnc2)cc1F ; COC1(c2ccccn2)CCCc2nc(-c3cc(F)c(O)c(F)c3)ccc21 ; Oc1c(F)cc(-c2ccc3c(n2)CCCC3(O)c2ccccn2)cc1F ; COC1(c2ncco2)CCCc2nc(-c3cc(F)c(O)c(F)c3)ccc21]

and pharmaceutically acceptable salts thereof.

In another aspect, disclosed herein is a pharmaceutical composition comprising a compound of formula (I), or a pharmaceutically acceptable salt thereof, and a pharmaceutically acceptable carrier.

In another aspect, disclosed herein is a method of treating cancer in a subject in need thereof, comprising administering to the subject a therapeutically effective amount of a compound of formula (I), or a pharmaceutically acceptable salt thereof. In some embodiments, the cancer is prostate cancer, breast cancer, or endometrial cancer. In some embodiments, the cancer is castration-resistant prostate cancer. In some embodiments, the method further comprises treating the subject with one or more additional therapies. In some embodiments, the one or more additional therapies are selected from surgery, chemotherapy, radiation therapy, hormone therapy, immunotherapy, cryotherapy, and thermotherapy, or any combination thereof.

In another aspect, disclosed herein is a method of inhibiting cancer cell proliferation, comprising contacting cancer cells with a compound of any one of claims 1-14, or a pharmaceutically acceptable salt thereof, in an amount effective to inhibit the cancer cell proliferation. In some embodiments, the cancer cells are prostate cancer cells, breast cancer cells, or endometrial cancer cells.

In another aspect, disclosed herein is a method of inhibiting the activity of 3β-hydroxysteroid dehydrogenase in a sample, comprising contacting the sample with a compound of any one of claims 1-14, or a pharmaceutically acceptable salt thereof, in an amount effective to inhibit the activity of 30-hydroxysteroid dehydrogenase.

In another aspects, disclosed herein is a method of treating cancer in a subject in need thereof, the method comprising determining that a cytosine nucleotide is present at position 1245 of the HSD3β1 gene or a threonine is present at position 367 of the 3βHSD1 protein by assaying a biological sample from the subject, and administering a therapeutically effective amount of a compound provided herein, or a pharmaceutically acceptable salt thereof, to the subject. In some embodiments, the cancer is prostate cancer, breast cancer, or endometrial cancer. In some embodiments, the biological sample comprises cancer cells.

Other aspects and embodiments will become apparent in light of the following description.

### BRIEF DESCRIPTION OF THE DRAWINGS

FIG. 1 shows the activity of a compound disclosed herein in a CRPC C4-2 flank tumor model in mice.

### DETAILED DESCRIPTION

Disclosed herein are compounds that inhibit the function of 38-hydroxysteroid dehydrogenase (3βHSD1). Also disclosed herein are pharmaceutical compositions comprising the compounds, and methods of using the compounds for the treatment of diseases dependent on the activity of 3βHSD1, including cancers such as prostate cancer, breast cancer, and endometrial cancer.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to +10% of the indicated number. For example, “about 10%” may indicate a range of 9% to 11%, and “about 1” may mean from 0.9-1.1. Other meanings of “about” may be apparent from the context, such as rounding off; for example, “about 1” may also mean from 0.5 to 1.4.

As used herein, the term “alkyl” refers to a radical of a straight or branched saturated hydrocarbon chain. The alkyl chain can include, e.g., from 1 to 24 carbon atoms (C₁-C₂₄ alkyl), 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 1 to 3 carbon atoms (C₁-C₃ alkyl), or 1 to 2 carbon atoms (C₁-C₂ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkenyl” refers to a radical of a straight or branched hydrocarbon chain containing at least one carbon-carbon double bond and no triple bonds (“alkenyl”; see below). The double bond(s) may be located at any position(s) with the hydrocarbon chain. The alkenyl chain can include, e.g., from 2 to 24 carbon atoms (C₂-C₂₄ alkenyl), 2 to 16 carbon atoms (C₂-C₁₆ alkenyl), 2 to 14 carbon atoms (C₂-C₁₄ alkenyl), 2 to 12 carbon atoms (C₂-C₁₂ alkenyl), 2 to 10 carbon atoms (C₂-C₁₀ alkenyl), 2 to 8 carbon atoms (C₂-C₈ alkenyl), 2 to 6 carbon atoms (C₂-C₆ alkenyl), 2 to 4 carbon atoms (C₂-C₄ alkenyl), 2 to 3 carbon atoms (C₂-C₃ alkenyl), or 2 carbon atoms (C₂ alkenyl). Representative examples of alkenyl include, but are not limited to, ethenyl, 1-propenyl, 2-propenyl, 1-butenyl, 2-butenyl, butadienyl, 2-methyl-2-propenyl, 3-butenyl, pentenyl, pentadienyl, hexenyl, heptenyl, octenyl, octatrienyl, and the like.

As used herein, the term “alkynyl” means a radical of a straight or branched hydrocarbon chain containing at least one carbon-carbon triple bond. The alkynyl chain can include, e.g., from 2 to 24 carbon atoms (C₂-C₂₄ alkynyl), 2 to 16 carbon atoms (C₂-C₁₆ alkynyl), 2 to 14 carbon atoms (C₂-C₁₄ alkynyl), 2 to 12 carbon atoms (C₂-C₁₂ alkynyl), 2 to 10 carbon atoms (C₂-C₁₀ alkynyl), 2 to 8 carbon atoms (C₂-C₅ alkynyl), 2 to 6 carbon atoms (C₂-C₆ alkynyl), 2 to 4 carbon atoms (C₂-C₄ alkynyl), 2 to 3 carbon atoms (C₂-C₃ alkynyl), or 2 carbon atoms (C₂ alkynyl). The triple bond(s) may be located at any position(s) with the hydrocarbon chain. Representative examples of alkynyl include, but are not limited to, ethynyl, 1-propynyl, 2-propynyl, 1-butynyl, 2-butynyl, and the like.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

As used herein, the term “amino” refers to a group —NRₓR_{y}, wherein Rₓ and R_{y} are selected from hydrogen and alkyl (e.g., C₁-C₄ alkyl).

As used herein, “aryl” refers to a radical of a monocyclic, bicyclic, or tricyclic 4n+2 aromatic ring system (e.g., having 6, 10, or 14π electrons shared in a cyclic array) having 6-14 ring carbon atoms and zero heteroatoms (“C₆-C₁₄ aryl”). In some embodiments, an aryl group has six ring carbon atoms (“C₆ aryl”; i.e., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracenyl and phenanthrenyl).

As used herein, the term “cycloalkyl” refers to a radical of a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “cyano” refers to a —CN group.

As used herein, the term “halogen” or “halo” refers to F, Cl, Br, or I.

The term “haloalkyl,” as used herein, refers to an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen. Representative examples of haloalkyl include, but are not limited to, fluoromethyl, difluoromethyl, trifluoromethyl, 2-fluoroethyl, 2,2,2-trifluoroethyl, and 3,3,3-trifluoropropyl.

The term “haloalkoxy,” as used herein, means a haloalkyl group, as defined herein, is appended to the parent molecular moiety through an oxygen atom. Representative examples of haloalkoxy include, but are not limited to, difluoromethoxy, trifluoromethoxy, and 2,2,2-trifluoroethoxy.

As used herein, “heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin-2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a Ce aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like.

Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

As used herein, the term “hydroxy” or “hydroxyl” refers to an —OH group.

As used herein, the term “nitro” refers to an —NO₂ group.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, thiol, thione, or combinations thereof.

As used herein, in chemical structures the indication:

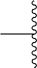

represents a point of attachment of one moiety to another moiety (e.g., a substituent group to the rest of the compound).

As used herein, the term “subject” generally refers to any vertebrate, including, but not limited to a mammal. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Treatment or diagnosis of humans is of particular interest. In some embodiments, the subject has or is at risk of having cancer.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### Compounds

Disclosed herein are compounds of formula (I):

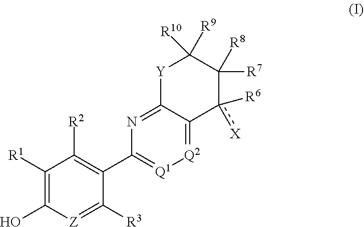

and pharmaceutically acceptable salts thereof, wherein:
R¹ is halo, cyano, or C₁-C₄ haloalkyl;
Z is selected from CR⁴ and N;
R², R³, and R⁴ are independently selected from hydrogen, halo, and C₁-C₄ alkyl;
Q¹ is CH or N;
Q₂ is CR⁵ or N;
R⁵ is selected from hydrogen, C₁-C₄ alkyl, halo, and C₁-C₄ haloalkyl;
--- represents the presence or absence of a bond, wherein, when --- represents the presence of a bond, X is oxo and R⁶ is absent; and wherein, when --- represents the absence of a bond, X is selected from —OR^{a} and —NR^{b}R^{c}, and R⁶ is selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, and aryl-C₁-C₄-alkyl;
Y is selected from —CH₂—, —NR^{d}—, —O—, —S—, —CH₂CH₂—, —NHCH₂—, —OCH₂—, —SCH₂—, and a bond;
R⁷, R⁸, R⁹, and R¹⁰ are each independently selected from hydrogen, C₁-C₆ alkyl, C₂-C₆ alkenyl, C₂-C₆ alkynyl, C₁-C₆ alkoxy, hydroxy, cyano, halo, C₃-C₆ cycloalkyl, heterocyclyl, aryl, heteroaryl, C₁-C₄-alkoxy-C₁-C₆-alkyl, hydroxy-C₁-C₆-alkyl, halo-C₁-C₆-alkyl, carboxy-C₁-C₆-alkyl, amino-C₁-C₆-alkyl, C₃-C₆-cycloalkyl-C₁-C₄-alkyl, aryl-C₁-C₄-alkyl, and heteroaryl-C₁-C₄-alkyl, wherein R⁷ and R⁸, together with the carbon atom to which they are attached, are optionally taken together to form a 3- to 6-membered ring; and R^{a}, R^{b}, R^{c}, and R^{d} are each independently selected from hydrogen, C₁-C₄ alkyl, C₁-C₄-alkoxy-C₁-C₄-alkyl, heterocyclyl, and heterocyclyl-C₁-C₄-alkyl;
wherein each cycloalkyl, heterocyclyl, aryl, and heteroaryl is independently unsubstituted or substituted with 1, 2, or 3 substituents independently selected from C₁-C₆ alkyl, C₁-C₆ alkoxy, hydroxy, cyano, halo, and C₅-C₆ cycloalkyl.

In some embodiments:
R¹ is halo or cyano;
Z is selected from CR⁴ and N;
R², R³, and R⁴ are independently selected from hydrogen, halo, and C₁-C₄ alkyl;
Q₁ is CH or N;
Q₂ is CR⁵ or N;
R⁵ is selected from hydrogen, C₁-C₄ alkyl, halo, and C₁-C₄ haloalkyl;
--- represents the presence or absence of a bond; wherein, when --- represents the presence of a bond, X is oxo and R⁶ is absent; and wherein, when --- represents the absence of a bond, X is selected from —OH and —NH₂, and R⁶ is selected from hydrogen, C₁-C₆ alkyl, C₂-C₆ alkenyl, C₂-C₆ alkynyl, C₁-C₆ haloalkyl, C₃-C₆ cycloalkyl, aryl, and aryl-C₁-C₄-alkyl;
Y is selected from —CH₂—, —NH—, —O—, —S—, —CH₂CH₂—, —NHCH₂—, —OCH₂—, —SCH₂—, and a bond; and
R⁷, R⁸, R⁹, and R¹⁰ are each independently selected from hydrogen, C₁-C₆ alkyl, C₂-C₆ alkenyl, C₂-C₆ alkynyl, C₁-C₆ alkoxy, hydroxy, cyano, halo, C₃-C₆ cycloalkyl, aryl, heteroaryl, C₁-C₄-alkoxy-C₁-C₆-alkyl, hydroxy-C₁-C₆-alkyl, halo-C₁-C₆-alkyl, carboxy-C₁-C₆-alkyl, C₃-C₆-cycloalkyl-C₁-C₄-alkyl, aryl-C₁-C₄-alkyl, and heteroaryl-C₁-C₄-alkyl, wherein R⁷ and R⁸, together with the carbon atom to which they are attached, are optionally taken together to form a 3- to 6-membered ring.

In some embodiments, R¹ is halo. In some embodiments, R¹ is fluoro. In some embodiments, R¹ is chloro. In some embodiments, R¹ is cyano. In some embodiments, R¹ is C₁-C₄ haloalkyl. In some embodiments, R¹ is trifluoromethyl. In some embodiments, R¹ is fluoro, chloro, cyano, or trifluoromethyl. In some embodiments, R¹ is fluoro or cyano.

In some embodiments, R² and R³ are each independently selected from hydrogen, halo, and C₁-C₂ alkyl. In some embodiments, R² and R³ are each independently selected from hydrogen, fluoro, and methyl. In some embodiments, R² and R³ are each independently selected from hydrogen and halo. In some embodiments, R² and R³ are each independently selected from hydrogen and fluoro. In some embodiments, R² and R³ are each hydrogen. In some embodiments, R² is hydrogen and R³ is halo. In some embodiments, R² is hydrogen and R³ is fluoro. In some embodiments, R² is halo and R³ is hydrogen. In some embodiments, R² is fluoro and R³ is hydrogen. In some embodiments, R² is C₁-C₄ alkyl and R³ is hydrogen. In some embodiments, R² is methyl and R³ is hydrogen. In some embodiments, R² and R³ are each halo. In some embodiments, R² and R³ are each fluoro. In some embodiments, R² is hydrogen. In some embodiments, R² is halo. In some embodiments, R² is fluoro. In some embodiments, R² is C₁-C₄ alkyl. In some embodiments, R² is methyl. In some embodiments, R³ is hydrogen. In some embodiments, R³ is halo. In some embodiments, R³ is fluoro.

In some embodiments, Z is CR⁴. In some embodiments, R⁴ is selected from hydrogen, fluoro, chloro, and methyl. In some embodiments, R⁴ is selected from hydrogen and halo. In some embodiments, R⁴ is selected from hydrogen, fluoro, and chloro. In some embodiments, R⁴ is selected from hydrogen and fluoro. In some embodiments, R⁴ is hydrogen. In some embodiments, R⁴ is halo. In some embodiments, R⁴ is fluoro. In some embodiments, R⁴ is chloro. In some embodiments, R⁴ is C₁-C₄ alkyl. In some embodiments, R⁴ is methyl. In some embodiments, Z is N.

In some embodiments, the group

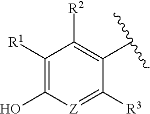

in formula (I) is selected from:

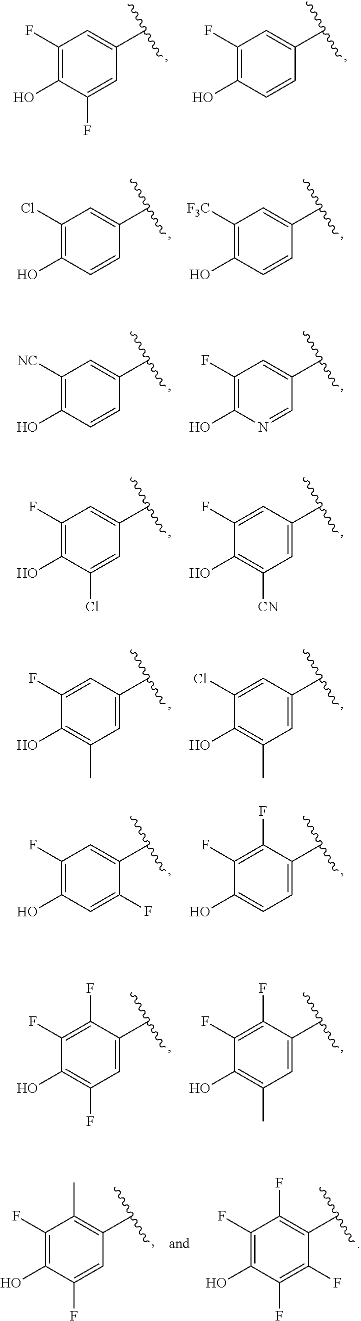

In some embodiments, the group

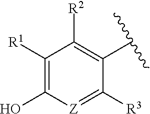

in formula (I) is selected from:

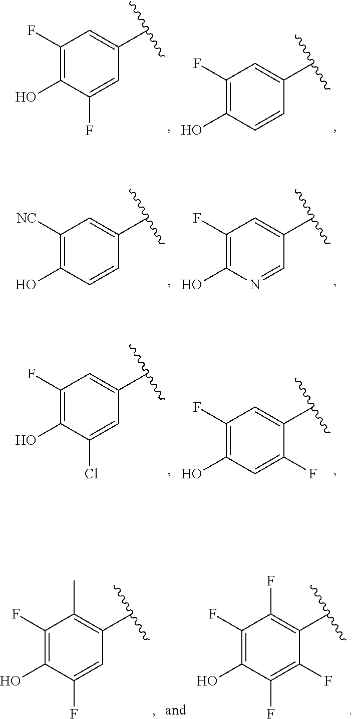

In some embodiments, the group

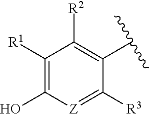

in formula (I) is:

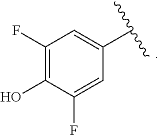 [SMILES: *c1cc(F)c(O)c(F)c1]

In some embodiments, Q₁ is CH or N. In some embodiments, Q₁ is CH. In some embodiments, Q¹ is N. In some embodiments, Q₂ is CR⁵, and R⁵ is hydrogen or C₁-C₄ alkyl. In some embodiments, Q₂ is CR⁵, and R⁵ is hydrogen, methyl, ethyl, or iso-propyl. In some embodiments, Q₂ is CR⁵, and R⁵ is hydrogen or methyl. In some embodiments, Q₂ is CR⁶, and R⁵ is hydrogen. In some embodiments, Q₂ is N. In some embodiments, Q₁ is CH, Q₂ is CR⁵, and R⁵ is hydrogen, methyl, ethyl, or iso-propyl. In some embodiments, Q₁ is CH, Q₂ is CR⁵, and R⁵ is hydrogen or methyl. In some embodiments, Q₁ is N, Q₂ is CR⁵, and R⁵ is hydrogen. In some embodiments, Q¹ is CH and Q₂ is N. In some embodiments, Q₁ is CH, Q₂ is CR⁵, and R⁵ is hydrogen.

In some embodiments, --- represents the presence of a bond, X is oxo, and R⁶ is absent.

In some embodiments, --- represents the absence of a bond, X is selected from —OR^{a} and —NR^{b}R^{c}, and R⁶ is selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, and aryl-C₁-C₄-alkyl. In some embodiments, ---represents the absence of a bond, X is selected from —OR^{a} and —NR^{b}R^{c}, and R⁶ is selected from hydrogen, C₁-C₃ alkyl, C₃-C₄ cycloalkyl, phenyl, a monocyclic 5- or 6-membered heteroaryl having 1 or 2 heteroatoms independently selected from N, O, and S, and phenyl-C₁-C₂-alkyl, wherein R^{a} is selected from hydrogen, C₁-C₃ alkyl, heterocyclyl, and heterocyclyl-C₁-C₄-alkyl, and R^{b} and R^{c} are each hydrogen, wherein the heterocyclyl is a monocyclic 4- to 6-membered heterocyclyl having one heteroatom selected from O and N. In some embodiments, --- represents the absence of a bond; X is selected from —OR^{a} and —NR^{b}R^{c}; and R^{c} is selected from hydrogen, methyl, ethyl, isopropyl, cyclopropyl, phenyl, pyridyl, oxazolyl, and benzyl, wherein R^{a} is selected from hydrogen, methyl, ethyl, oxetanyl, and —CH₂-oxetanyl, and R^{b} and R^{c} are each hydrogen.

In some embodiments, --- represents the absence of a bond, X is selected from —OH and —NH₂, and R⁶ is selected from hydrogen, C₁-C₆ alkyl, C₂-C₆ alkenyl, C₂-C₆ alkynyl, C₁-C₆ haloalkyl, C₃-C₆ cycloalkyl, aryl, and aryl-C₁-C₄-alkyl. In some embodiments, --- represents the absence of a bond; X is selected from —OH and —NH₂; and R⁶ is selected from hydrogen, C₁-C₃ alkyl, C₃-C₄ cycloalkyl, phenyl, and phenyl-C₁-C₂-alkyl. In some embodiments, --- represents the absence of a bond; X is —OH; and R⁶ is selected from hydrogen, methyl, cyclopropyl, phenyl, and benzyl. In some embodiments, --- represents the absence of a bond; X is —NH₂; and R⁶ is hydrogen.

In some embodiments, Y is selected from —CH₂—, —NR^{d}—, —O—, —CH₂CH₂—, and a bond, wherein R^{d} is selected from hydrogen, C₁-C₄ alkyl, and C₁-C₄-alkoxy-C₁-C₄-alkyl. In some embodiments, Y is selected from —CH₂—, —NR^{d}—, —O—, —CH₂CH₂—, and a bond, wherein R^{d} is selected from hydrogen, C₁-C₂ alkyl, and C₁-C₂-alkoxy-C₁-C₂-alkyl. In some embodiments, Y is selected from —CH₂—, —NR^{d}—, —O—, —CH₂CH₂—, and a bond, wherein R^{d} is selected from hydrogen, methyl, ethyl, and —CH₂CH₂OCH₃. In some embodiments, Y is selected from —CH₂—, —NH—, —CH₂CH₂—, and a bond. In some embodiments, Y is —CH₂—. In some embodiments, Y is —NH—. In some embodiments, Y is —N(CH₃)—. In some embodiments, Y is —N(CH₂CH₃)—. In some embodiments, Y is —N(CH₂CH₂OCH₃)—. In some embodiments, Y is —CH₂CH₂—. In some embodiments, Y is —O—. In some embodiments, Y is a bond.

In some embodiments, R⁷ and R⁸ are each independently selected from hydrogen, C₁-C₆ alkyl, C₂-C₆ alkenyl, C₂-C₆ alkynyl, C₁-C₆ alkoxy, hydroxy, cyano, halo, C₃-C₆ cycloalkyl, heterocyclyl, aryl, heteroaryl, C₁-C₄-alkoxy-C₁-C₆-alkyl, hydroxy-C₁-C₆-alkyl, halo-C₁-C₆-alkyl, carboxy-C₁-C₆-alkyl, amino-C₁-C₆-alkyl, C₃-C₆-cycloalkyl-C₁-C₄-alkyl, aryl-C₁-C₄-alkyl, and heteroaryl-C₁-C₄-alkyl. In some embodiments, R⁷ and R⁸ are each independently selected from hydrogen, C₁-C₃ alkyl, hydroxy, cyano, halo, C₃-C₆ cycloalkyl, monocyclic 3- to 6-membered heterocyclyl having one heteroatom selected from O and N, aryl, C₁-C₃-alkoxy-C₁-C₃-alkyl, hydroxy-C₁-C₃-alkyl, halo-C₁-C₃-alkyl, carboxy-C₁-C₃-alkyl, amino-C₁-C₃-alkyl, C₃-C₄-cycloalkyl-C₁-C₃-alkyl, aryl-C₁-C₃-alkyl, and heteroaryl-C₁-C₃-alkyl, wherein the cycloalkyl and the heterocyclyl are each independently unsubstituted or substituted with one substituent selected from hydroxy, methyl, halo, and cyano. In some embodiments, R⁷ and R⁸ are each independently selected from hydrogen, C₁-C₃ alkyl, hydroxy, cyano, halo, monocyclic 4-5-membered heterocyclyl having one O atom, aryl, methoxy-C₁-C₂-alkyl, hydroxy-C₁-C₃-alkyl, halo-C₁-C₃-alkyl, carboxy-C₁-C₃-alkyl, amino-C₁-C₂-alkyl, C₃-C₄-cycloalkyl-C₁-C₂-alkyl, aryl-C₁-C₂-alkyl, and heteroaryl-C₁-C₂-alkyl, wherein the heterocyclyl is unsubstituted or substituted with one hydroxy group. In some embodiments, R⁷ and R⁸ are each independently selected from hydrogen, methyl, ethyl, hydroxy, cyano, fluoro, 3-hydroxyoxetan-3-yl, phenyl, benzyl, —CH₂OH, —CF₃, —CF₂CF₃, —CH₂CF₃, —CH₂N(CH₃)₂, —CH₂OCH₃, —CH₂-cyclopropyl, and —CH₂-pyridyl.

In some embodiments, R⁷ and R⁸ are each independently selected from hydrogen, C₁-C₆ alkyl, C₂-C₆ alkenyl, C₂-C₆ alkynyl, C₁-C₆ alkoxy, hydroxy, cyano, halo, C₃-C₆ cycloalkyl, aryl, heteroaryl, C₁-C₄-alkoxy-C₁-C₆-alkyl, hydroxy-C₁-C₆-alkyl, halo-C₁-C₆-alkyl, carboxy-C₁-C₆-alkyl, C₃-C₆-cycloalkyl-C₁-C₄-alkyl, aryl-C₁-C₄-alkyl, and heteroaryl-C₁-C₄-alkyl. In some embodiments, R⁷ and R⁸ are each independently selected from hydrogen, C₁-C₃ alkyl, hydroxy, cyano, halo, C₃-C₆ cycloalkyl, aryl, halo-C₁-C₃-alkyl, carboxy-C₁-C₃-alkyl, C₃-C₄-cycloalkyl-C₁-C₂-alkyl, aryl-C₁-C₃-alkyl, and heteroaryl-C₁-C₂-alkyl. In some embodiments, R⁷ and R⁸ are each independently selected from hydrogen, C₁-C₃ alkyl, aryl, C₃-C₄-cycloalkyl-C₁-C₃-alkyl, and aryl-C₁-C₂-alkyl. In some embodiments, R⁷ and R⁸ are each independently selected from hydrogen, methyl, ethyl, phenyl, benzyl, and —CH₂-cyclopropyl.

In some embodiments, R⁷ and R⁸, together with the carbon atom to which they are attached, are taken together to form a 3- to 6-membered cycloalkyl or a 3- to 6-membered monocyclic heterocyclyl having one or two heteroatoms independently selected from O and N. In some embodiments, R⁷ and R⁸, together with the carbon atom to which they are attached, are taken together to form a 3- to 6-membered cycloalkyl or a 3- to 6-membered monocyclic heterocyclyl having one or two oxygen atoms. In some embodiments, R⁷ and R², together with the carbon atom to which they are attached, are taken together to form a ring selected from cyclopropyl, cyclobutyl, oxetane and dioxetane. In some embodiments, R⁷ and R³, together with the carbon atom to which they are attached, are taken together to form a 3- to 6-membered cycloalkyl. In some embodiments, R⁷ and R⁸, together with the carbon atom to which they are attached, are taken together to form a 3- or 4-membered cycloalkyl (i.e., cyclopropyl or cyclobutyl). In some embodiments, R⁷ and R⁸, together with the carbon atom to which they are attached, are taken together to form a 3- to 6-membered monocyclic heterocyclyl having one or two heteroatoms independently selected from O and N. In some embodiments, R⁷ and R⁸, together with the carbon atom to which they are attached, are taken together to form a 3- to 6-membered monocyclic heterocyclyl having one or two oxygen atoms. In some embodiments, R⁷ and R⁸, together with the carbon atom to which they are attached, are taken together to form an oxetane or dioxetane ring.

In some embodiments, R⁹ and R¹⁰ are each independently selected from hydrogen and C₁-C₆ alkyl. In some embodiments, R⁹ and R¹⁰ are each independently selected from hydrogen and C₁-C₄ alkyl. In some embodiments, R⁹ and R¹⁰ are each independently selected from hydrogen and methyl. In some embodiments, R⁹ and R¹⁰ are each hydrogen. In some embodiments, R⁹ and R¹⁰ are each methyl.

In some embodiments, the group

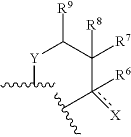

in compounds of formula (I) is selected from:

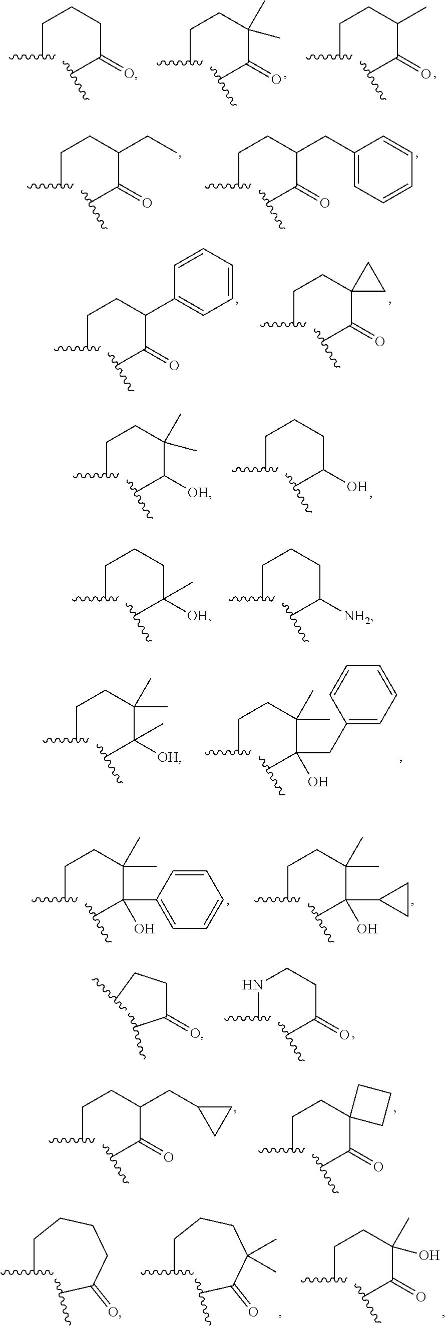

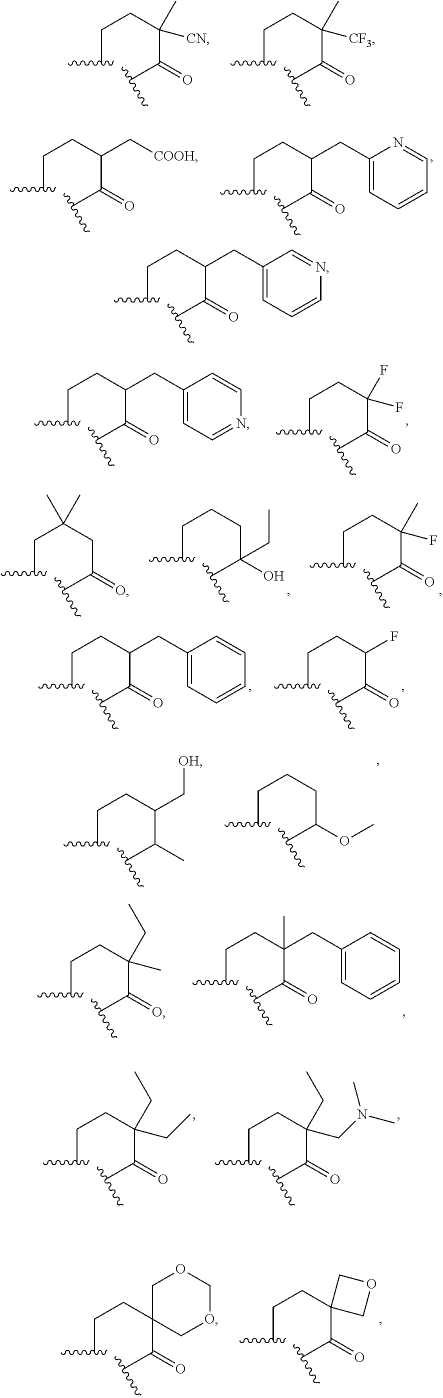

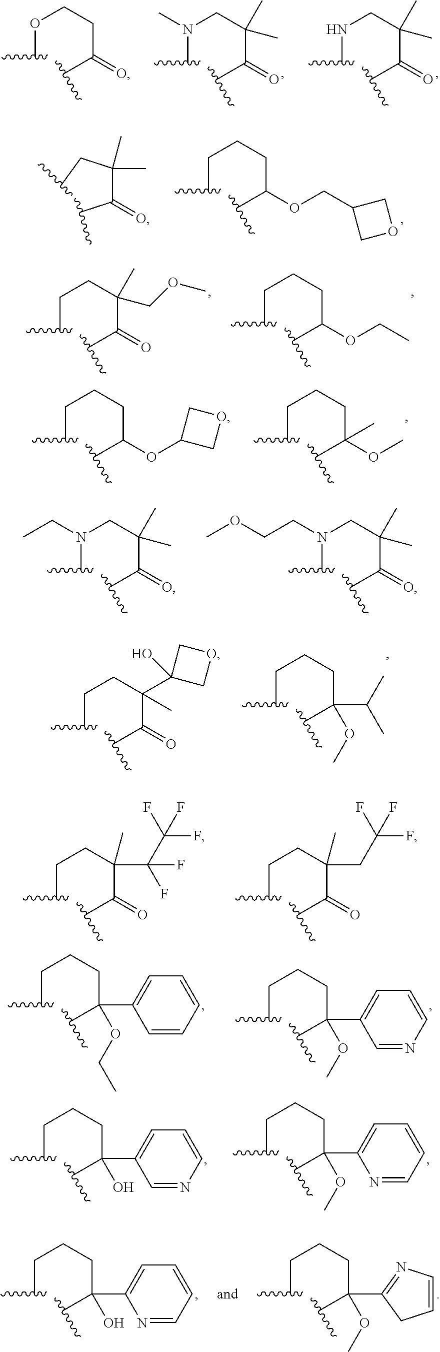

In some embodiments, the group

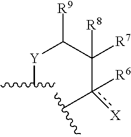

in compounds of formula (I) is selected from:

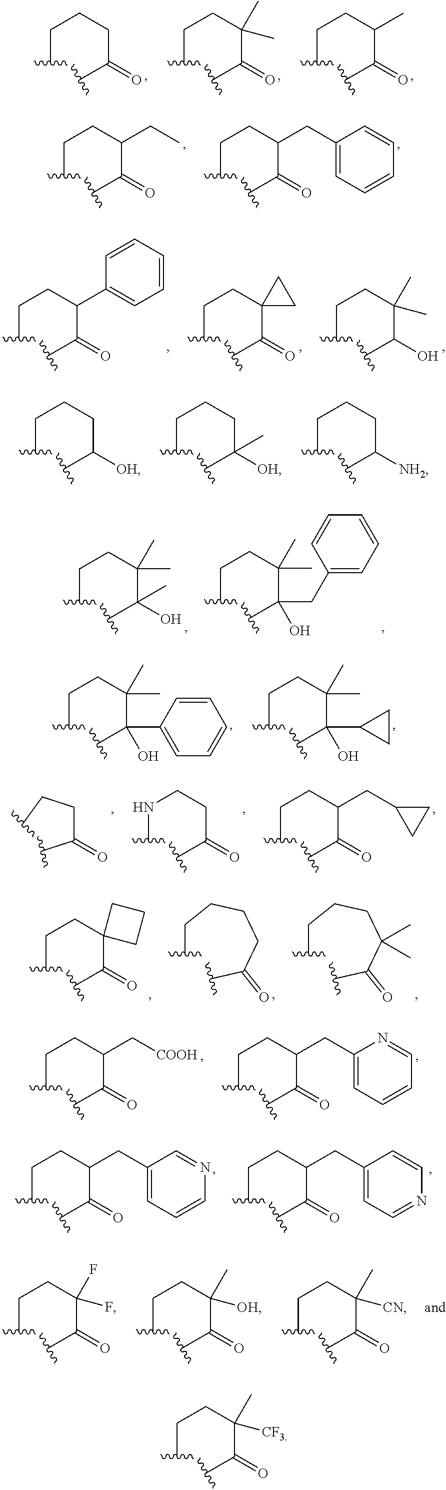

In some embodiments, the compound is selected from the group consisting of:

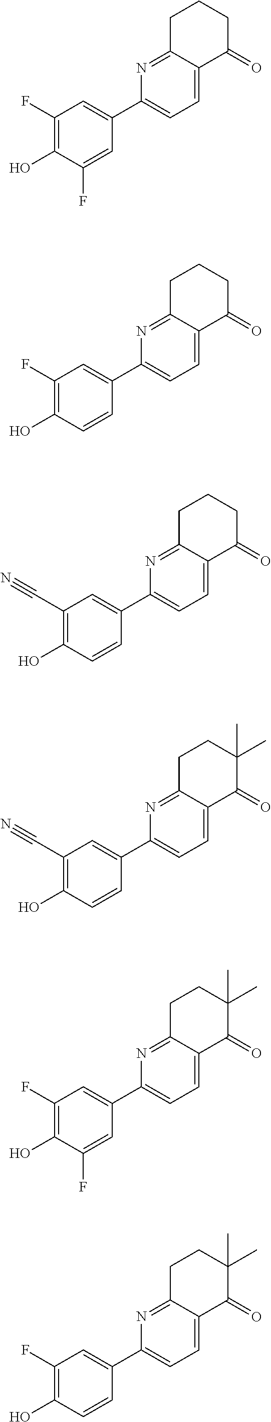 [SMILES: O=C1CCCc2nc(-c3cc(F)c(O)c(F)c3)ccc21 ; O=C1CCCc2nc(-c3ccc(O)c(F)c3)ccc21 ; N#Cc1cc(-c2ccc3c(n2)CCCC3=O)ccc1O ; CC1(C)CCc2nc(-c3ccc(O)c(C#N)c3)ccc2C1=O ; CC1(C)CCc2nc(-c3cc(F)c(O)c(F)c3)ccc2C1=O ; CC1(C)CCc2nc(-c3ccc(O)c(F)c3)ccc2C1=O]

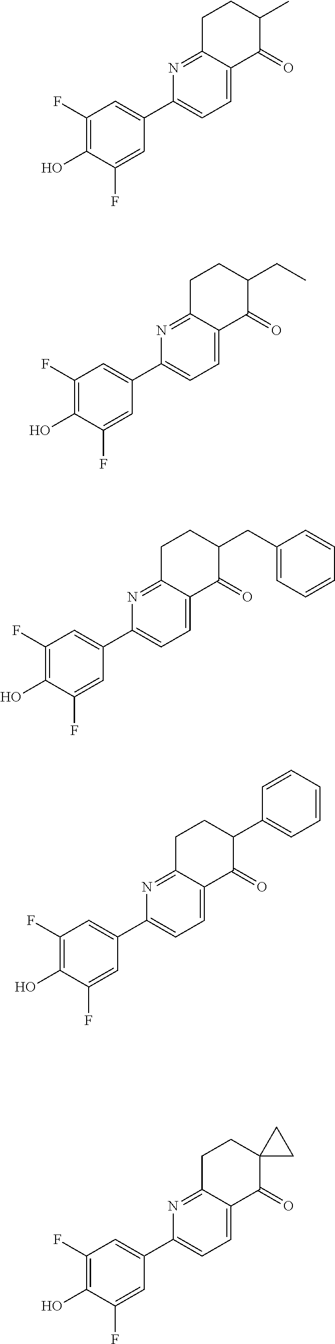 [SMILES: CC1CCc2nc(-c3cc(F)c(O)c(F)c3)ccc2C1=O ; CCC1CCc2nc(-c3cc(F)c(O)c(F)c3)ccc2C1=O ; O=C1c2ccc(-c3cc(F)c(O)c(F)c3)nc2CCC1Cc1ccccc1 ; O=C1c2ccc(-c3cc(F)c(O)c(F)c3)nc2CCC1c1ccccc1 ; O=C1c2ccc(-c3cc(F)c(O)c(F)c3)nc2CCC12CC2]

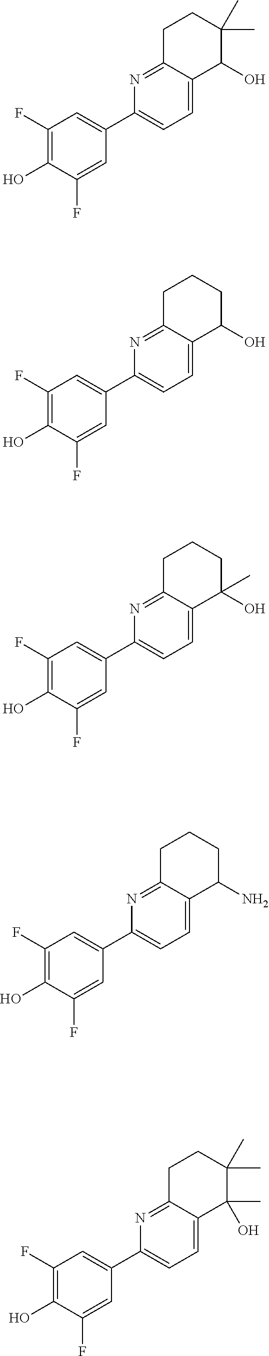 [SMILES: CC1(C)CCc2nc(-c3cc(F)c(O)c(F)c3)ccc2C1O ; Oc1c(F)cc(-c2ccc3c(n2)CCCC3O)cc1F ; CC1(O)CCCc2nc(-c3cc(F)c(O)c(F)c3)ccc21 ; NC1CCCc2nc(-c3cc(F)c(O)c(F)c3)ccc21 ; CC1(C)CCc2nc(-c3cc(F)c(O)c(F)c3)ccc2C1(C)O]

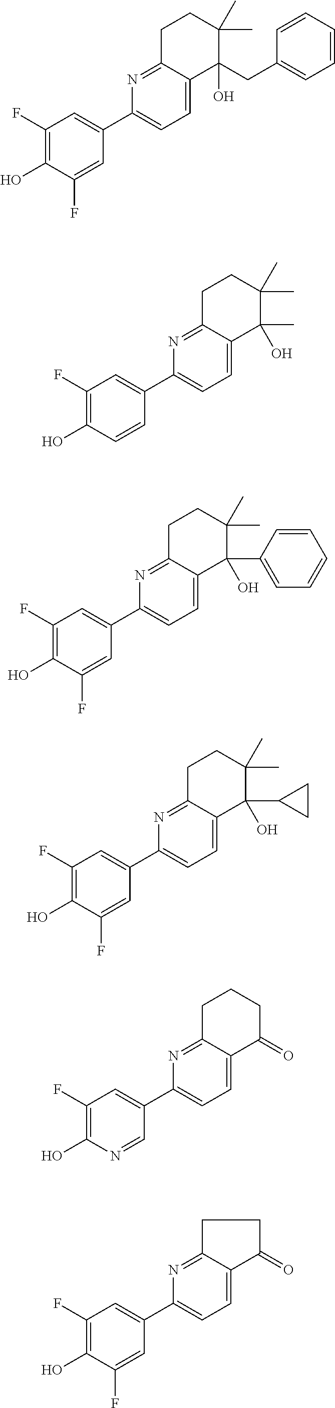 [SMILES: CC1(C)CCc2nc(-c3cc(F)c(O)c(F)c3)ccc2C1(O)Cc1ccccc1 ; CC1(C)CCc2nc(-c3ccc(O)c(F)c3)ccc2C1(C)O ; CC1(C)CCc2nc(-c3cc(F)c(O)c(F)c3)ccc2C1(O)c1ccccc1 ; CC1(C)CCc2nc(-c3cc(F)c(O)c(F)c3)ccc2C1(O)C1CC1 ; O=C1CCCc2nc(-c3cnc(O)c(F)c3)ccc21 ; O=C1CCc2nc(-c3cc(F)c(O)c(F)c3)ccc21]

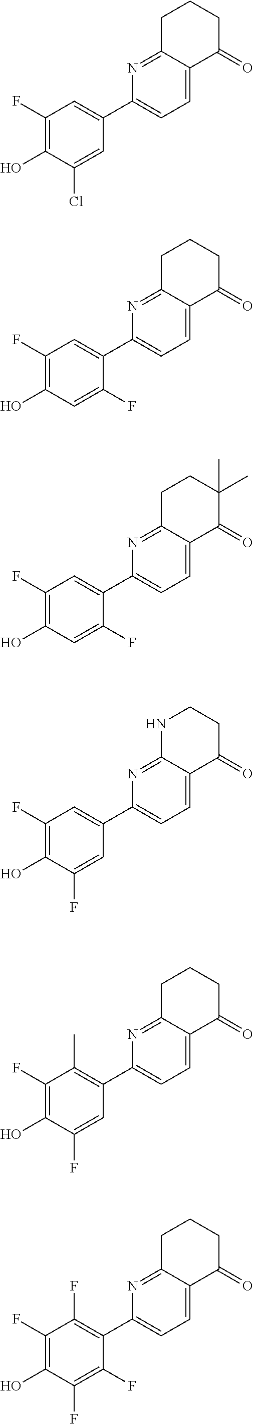 [SMILES: O=C1CCCc2nc(-c3cc(F)c(O)c(Cl)c3)ccc21 ; O=C1CCCc2nc(-c3cc(F)c(O)cc3F)ccc21 ; CC1(C)CCc2nc(-c3cc(F)c(O)cc3F)ccc2C1=O ; O=C1CCNc2nc(-c3cc(F)c(O)c(F)c3)ccc21 ; Cc1c(-c2ccc3c(n2)CCCC3=O)cc(F)c(O)c1F ; O=C1CCCc2nc(-c3c(F)c(F)c(O)c(F)c3F)ccc21]

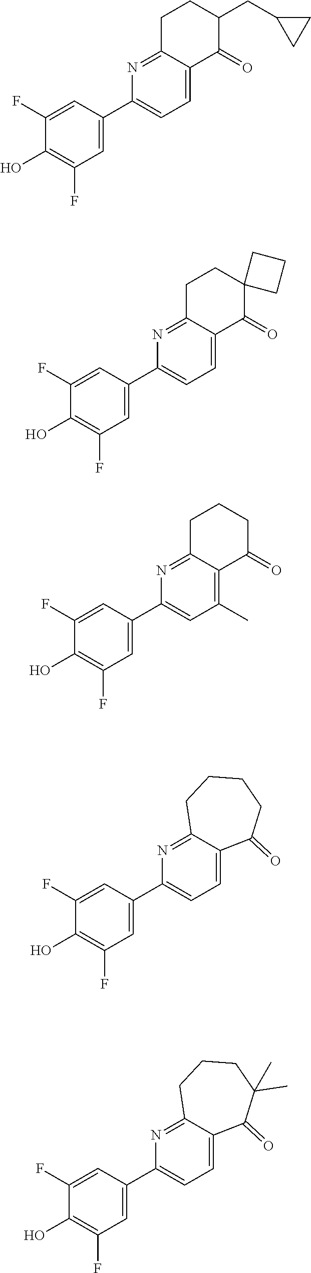 [SMILES: O=C1c2ccc(-c3cc(F)c(O)c(F)c3)nc2CCC1CC1CC1 ; O=C1c2ccc(-c3cc(F)c(O)c(F)c3)nc2CCC12CCC2 ; Cc1cc(-c2cc(F)c(O)c(F)c2)nc2c1C(=O)CCC2 ; O=C1CCCCc2nc(-c3cc(F)c(O)c(F)c3)ccc21 ; CC1(C)CCCc2nc(-c3cc(F)c(O)c(F)c3)ccc2C1=O]

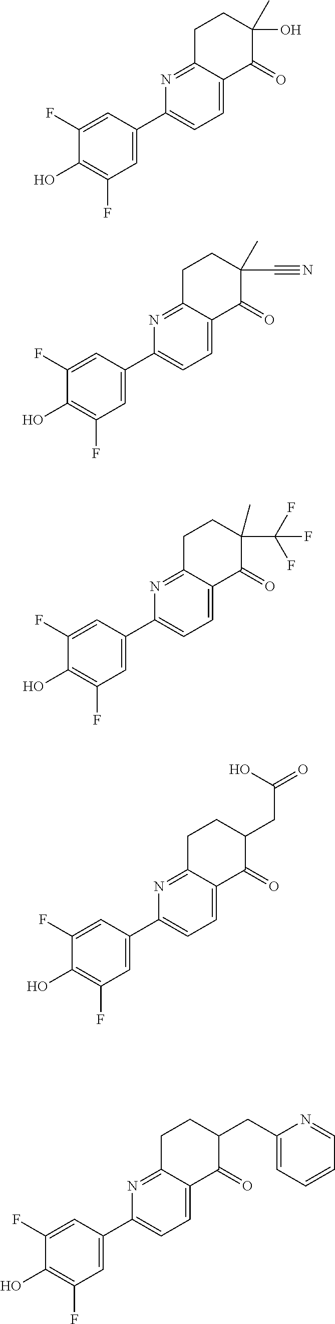 [SMILES: CC1(O)CCc2nc(-c3cc(F)c(O)c(F)c3)ccc2C1=O ; CC1(C#N)CCc2nc(-c3cc(F)c(O)c(F)c3)ccc2C1=O ; CC1(C(F)(F)F)CCc2nc(-c3cc(F)c(O)c(F)c3)ccc2C1=O ; O=C(O)CC1CCc2nc(-c3cc(F)c(O)c(F)c3)ccc2C1=O ; O=C1c2ccc(-c3cc(F)c(O)c(F)c3)nc2CCC1Cc1ccccn1]

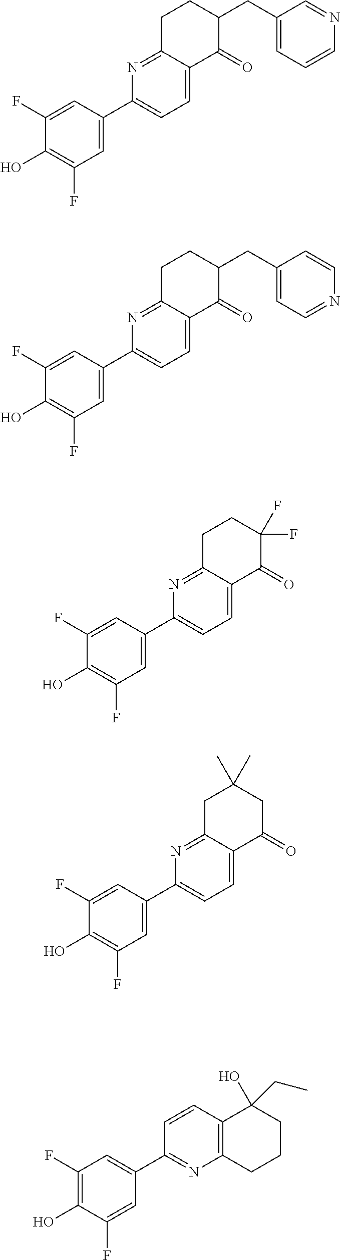 [SMILES: O=C1c2ccc(-c3cc(F)c(O)c(F)c3)nc2CCC1Cc1cccnc1 ; O=C1c2ccc(-c3cc(F)c(O)c(F)c3)nc2CCC1Cc1ccncc1 ; O=C1c2ccc(-c3cc(F)c(O)c(F)c3)nc2CCC1(F)F ; CC1(C)CC(=O)c2ccc(-c3cc(F)c(O)c(F)c3)nc2C1 ; CCC1(O)CCCc2nc(-c3cc(F)c(O)c(F)c3)ccc21]

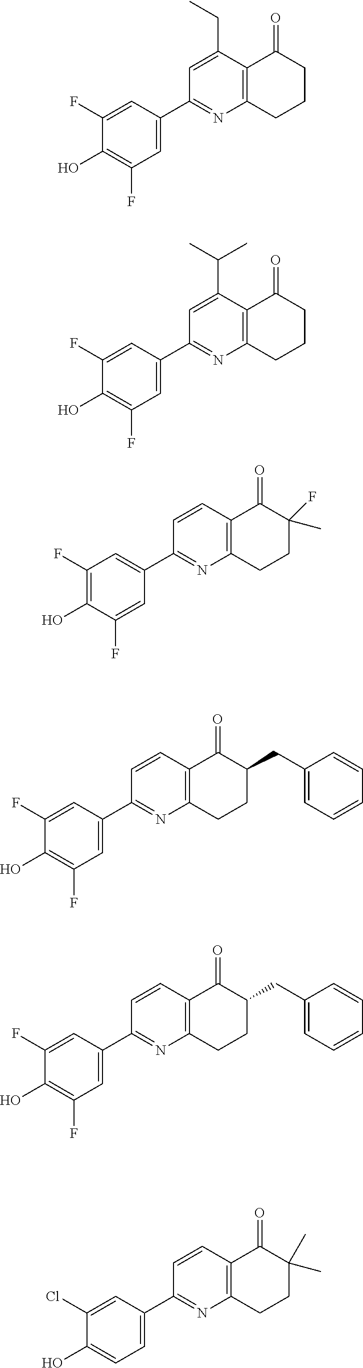 [SMILES: CCc1cc(-c2cc(F)c(O)c(F)c2)nc2c1C(=O)CCC2 ; CC(C)c1cc(-c2cc(F)c(O)c(F)c2)nc2c1C(=O)CCC2 ; CC1(F)CCc2nc(-c3cc(F)c(O)c(F)c3)ccc2C1=O ; O=C1c2ccc(-c3cc(F)c(O)c(F)c3)nc2CC[C@@H]1Cc1ccccc1 ; O=C1c2ccc(-c3cc(F)c(O)c(F)c3)nc2CC[C@H]1Cc1ccccc1 ; CC1(C)CCc2nc(-c3ccc(O)c(Cl)c3)ccc2C1=O]

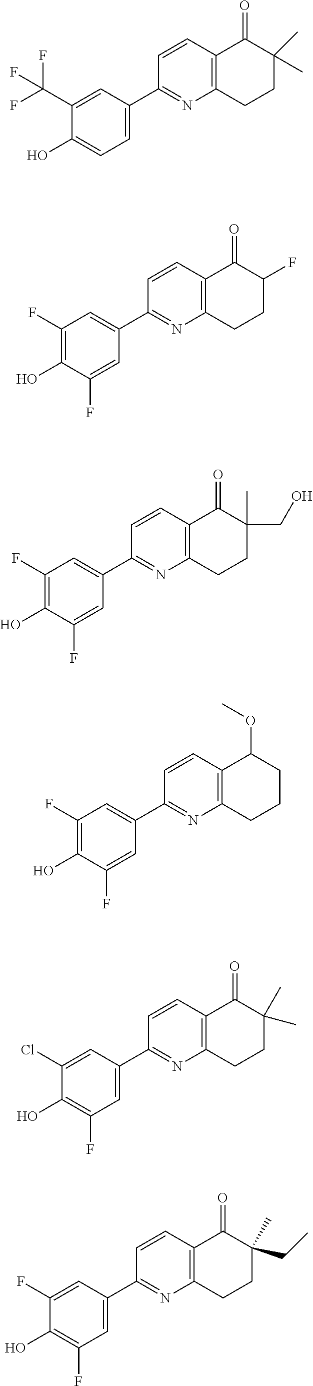 [SMILES: CC1(C)CCc2nc(-c3ccc(O)c(C(F)(F)F)c3)ccc2C1=O ; O=C1c2ccc(-c3cc(F)c(O)c(F)c3)nc2CCC1F ; CC1(CO)CCc2nc(-c3cc(F)c(O)c(F)c3)ccc2C1=O ; COC1CCCc2nc(-c3cc(F)c(O)c(F)c3)ccc21 ; CC1(C)CCc2nc(-c3cc(F)c(O)c(Cl)c3)ccc2C1=O ; CC[C@@]1(C)CCc2nc(-c3cc(F)c(O)c(F)c3)ccc2C1=O]

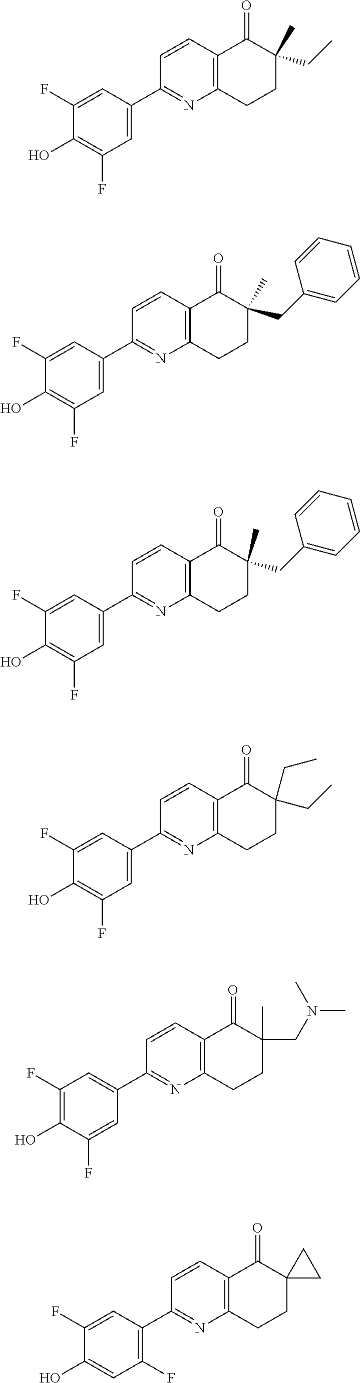 [SMILES: CC[C@]1(C)CCc2nc(-c3cc(F)c(O)c(F)c3)ccc2C1=O ; C[C@]1(Cc2ccccc2)CCc2nc(-c3cc(F)c(O)c(F)c3)ccc2C1=O ; C[C@@]1(Cc2ccccc2)CCc2nc(-c3cc(F)c(O)c(F)c3)ccc2C1=O ; CCC1(CC)CCc2nc(-c3cc(F)c(O)c(F)c3)ccc2C1=O ; CN(C)CC1(C)CCc2nc(-c3cc(F)c(O)c(F)c3)ccc2C1=O ; O=C1c2ccc(-c3cc(F)c(O)cc3F)nc2CCC12CC2]

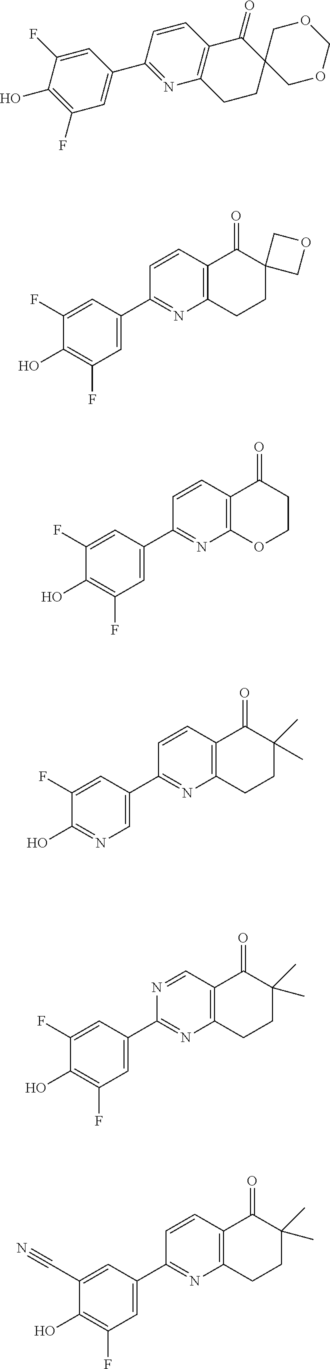 [SMILES: O=C1c2ccc(-c3cc(F)c(O)c(F)c3)nc2CCC12COCOC2 ; O=C1c2ccc(-c3cc(F)c(O)c(F)c3)nc2CCC12COC2 ; O=C1CCOc2nc(-c3cc(F)c(O)c(F)c3)ccc21 ; CC1(C)CCc2nc(-c3cnc(O)c(F)c3)ccc2C1=O ; CC1(C)CCc2nc(-c3cc(F)c(O)c(F)c3)ncc2C1=O ; CC1(C)CCc2nc(-c3cc(F)c(O)c(C#N)c3)ccc2C1=O]

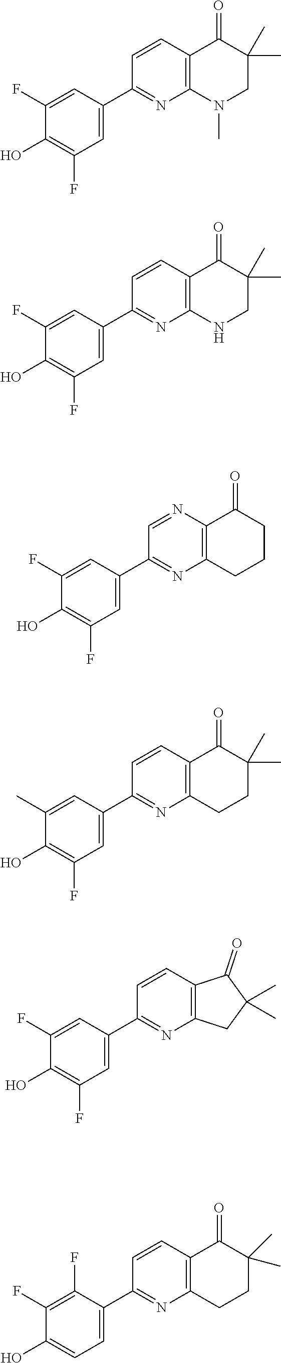

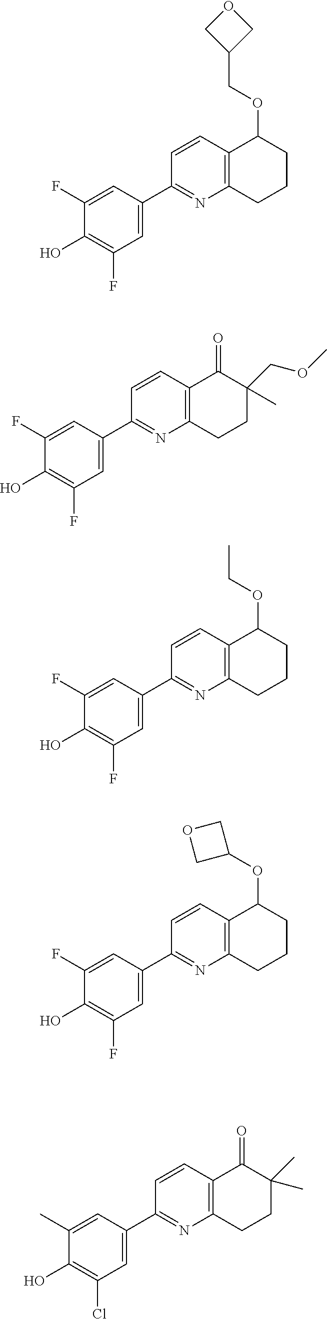

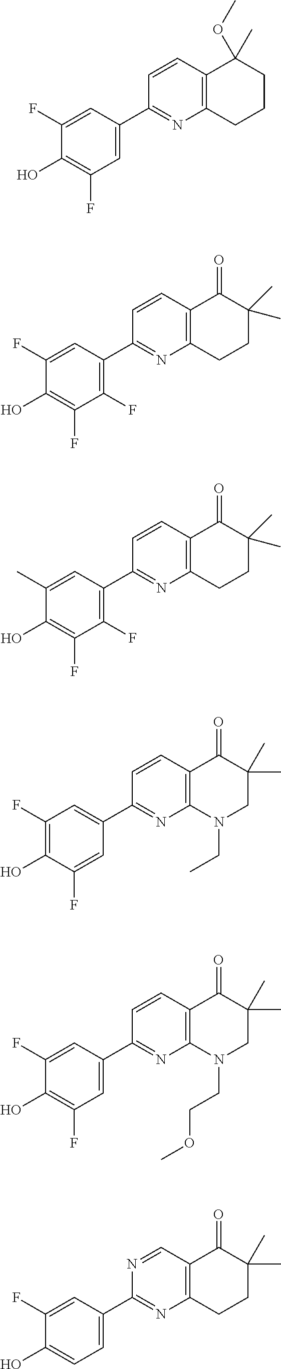

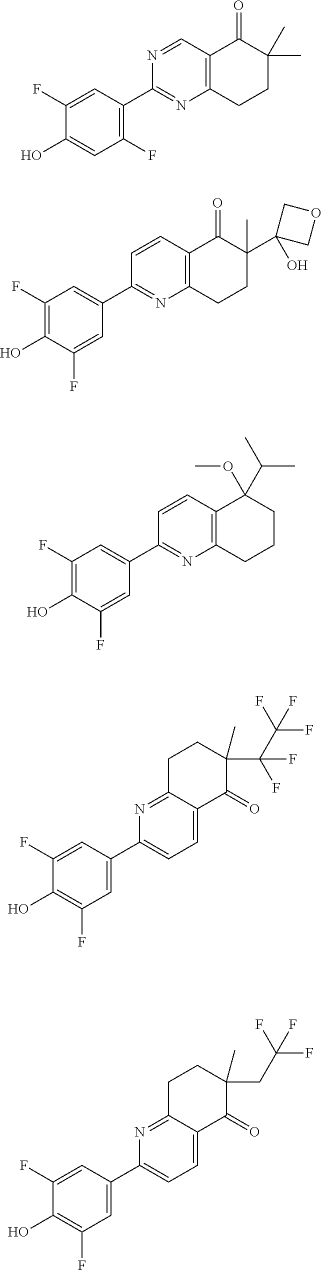 [SMILES: CC1(C)CCc2nc(-c3cc(F)c(O)cc3F)ncc2C1=O ; CC1(C2(O)COC2)CCc2nc(-c3cc(F)c(O)c(F)c3)ccc2C1=O ; COC1(C(C)C)CCCc2nc(-c3cc(F)c(O)c(F)c3)ccc21 ; CC1(C(F)(F)C(F)(F)F)CCc2nc(-c3cc(F)c(O)c(F)c3)ccc2C1=O ; CC1(CC(F)(F)F)CCc2nc(-c3cc(F)c(O)c(F)c3)ccc2C1=O]

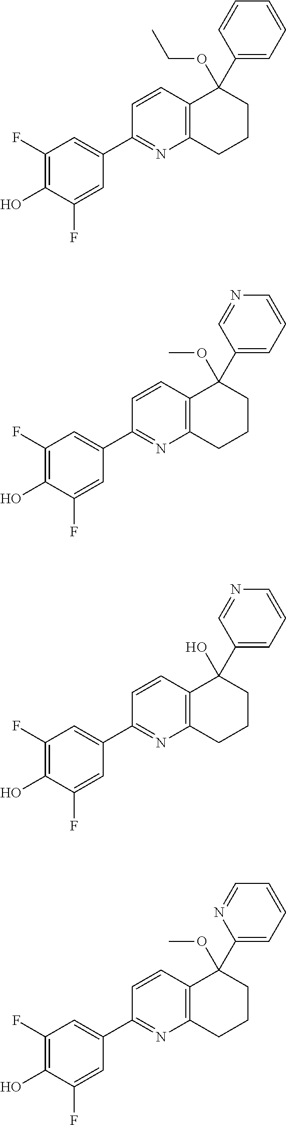 [SMILES: CCOC1(c2ccccc2)CCCc2nc(-c3cc(F)c(O)c(F)c3)ccc21 ; COC1(c2cccnc2)CCCc2nc(-c3cc(F)c(O)c(F)c3)ccc21 ; Oc1c(F)cc(-c2ccc3c(n2)CCCC3(O)c2cccnc2)cc1F ; COC1(c2ccccn2)CCCc2nc(-c3cc(F)c(O)c(F)c3)ccc21]

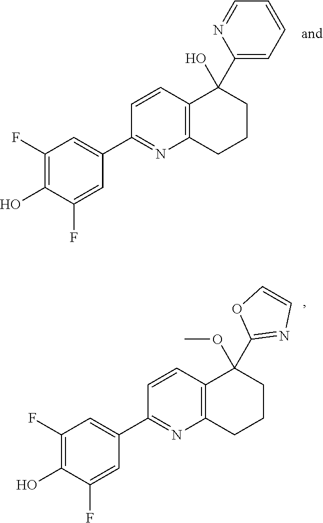 [SMILES: Oc1c(F)cc(-c2ccc3c(n2)CCCC3(O)c2ccccn2)cc1F ; COC1(c2ncco2)CCCc2nc(-c3cc(F)c(O)c(F)c3)ccc21]

and pharmaceutically acceptable salts thereof.

Certain compounds described herein have at least one asymmetric center. Additional asymmetric centers may be present depending upon the nature of the various substituents on the molecule. Compounds with asymmetric centers give rise to enantiomers (optical isomers), diastereomers (configurational isomers) or both, and it is intended that all of the possible enantiomers and diastereomers in mixtures and as pure or partially purified compounds are included within the scope of this invention. The present disclosure is meant to encompass all such isomeric forms of these compounds.

The independent syntheses of the enantiomerically or diastereomerically enriched compounds, or their chromatographic separations, may be achieved as known in the art by appropriate modification of the methodology disclosed herein. Their absolute stereochemistry may be determined by the x-ray crystallography of crystalline products or crystalline intermediates that are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods using chiral stationary phases, which methods are well known in the art. Alternatively, any enantiomer of a compound may be obtained by stereoselective synthesis using optically pure starting materials or reagents of known configuration by methods well known in the art.

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²p, ³⁵S, ¹⁸F, and ³⁶Cl, respectively. Substitution with heavier isotopes such as deuterium (²H) can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

a. Methods of Synthesis

Compounds disclosed heroin can be prepared by a variety of methods. One approach is illustrated in Scheme 1. Starting from boronic acid 1.1 (e.g., difluorophenol boronic acid, when each R is fluoro) and 2-chloropyridine tetralone 1.2, a Suzuki reaction in an ether solvent with heat provides final examples of type 1.3.

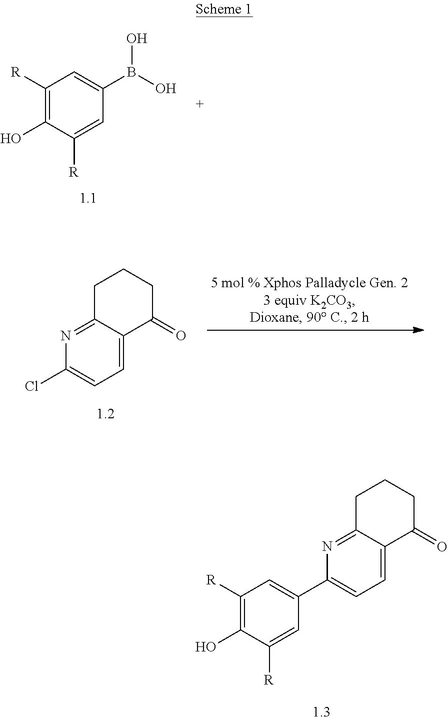

Compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Reactions can be worked up in a conventional manner, e.g., by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Standard experimentation, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4^{th} ed.), John Wiley & Sons, NY (2006).

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the procedures described herein using a pure geometric isomer as a starting material, or by resolution of a mixture of the geometric isomers of the compound or intermediates using a standard procedure such as chromatographic separation.

The synthetic schemes and specific examples as described are illustrative and are not to be read as limiting the scope of the disclosure or the claims. Alternatives, modifications, and equivalents of the synthetic methods and specific examples are contemplated.

b. Pharmaceutically Acceptable Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compound with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. Amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### Pharmaceutical Compositions

The disclosed compounds may be incorporated into pharmaceutical compositions suitable for administration to a subject (such as a patient, which may be a human or non-human). The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the disclosure are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease or condition, the prophylactically effective amount will be less than the therapeutically effective amount.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their pharmaceutically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90% by weight of the composition.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of Theobroma. The amount of lubricant(s) in a systemic or topical composition is typically about 5 to about 10% by weight of the composition.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50% by weight of the composition.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10% by weight of the composition.

Suitable colorants include a colorant such as an FD&C dye. When used, the amount of colorant in a systemic or topical composition is typically about 0.005 to about 0.1% by weight of the composition.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s), when used, in a systemic or topical composition is typically about 0.001 to about 1% by weight of the composition.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5% by weight of the composition.

Suitable preservatives include benzalkonium chloride, methyl paraben, and sodium benzoate. The amount of preservative(s) in a systemic or topical composition is typically about 0.01 to about 5% by weight of the composition.

Suitable glidants include silicon dioxide. The amount of glidant(s) in a systemic or topical composition is typically about 1 to about 5% by weight of the composition.

Suitable solvents include water, isotonic saline, ethyl oleate, glycerin, hydroxylated castor oils, alcohols such as ethanol, and phosphate buffer solutions. The amount of solvent(s) in a systemic or topical composition is typically from about 0 to about 100% by weight of the composition.

Suitable suspending agents include AVICEL RC-591 (from FMC Corporation of Philadelphia, PA) and sodium alginate. The amount of suspending agent(s) in a systemic or topical composition is typically about 1 to about 8% by weight of the composition.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5% by weight of the composition.

Although the amounts of components in the systemic compositions may vary depending on the type of systemic composition prepared, in general, systemic compositions include 0.01% to 50% by weight of an active compound and 50% to 99.99% by weight of one or more carriers. Compositions for parenteral administration typically include 0.1% to 10% by weight of actives and 90% to 99.9% by weight of a carrier including a diluent and a solvent.

Compositions for oral administration can have various dosage forms. For example, solid forms include tablets, capsules, granules, and bulk powders. These oral dosage forms include a safe and effective amount, usually at least about 5% by weight, and more particularly from about 25% to about 50% by weight of actives. The oral dosage compositions include about 50% to about 95% by weight of carriers, and more particularly, from about 50% to about 75% by weight.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this disclosure.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), or a pharmaceutically acceptable salt thereof), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this disclosure are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, Arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95% by weight of the composition.

Suitable propellants include propane, butane, isobutane, dimethyl ether, carbon dioxide, nitrous oxide, and combinations thereof. The amount of propellant(s) in a topical composition is typically about 0% to about 95% by weight of the composition.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95% by weight of the composition.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95% by weight of the composition.

The amount of thickener(s) in a topical composition is typically about 0% to about 95% by weight of the composition.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95% by weight of the composition.

The amount of fragrance in a topical composition is typically about 0% to about 0.5%, particularly, about 0.001% to about 0.1% by weight of the composition.

Suitable pH adjusting additives include HCl or NaOH in amounts sufficient to adjust the pH of a topical pharmaceutical composition.

### Methods of Use

As described herein, the disclosed compounds inhibit the function of 3βHSD1. Accordingly, the compounds and pharmaceutical compositions comprising the compounds can be used for the treatment of disorders that would benefit from inhibition of 3βHSD1, such as cancer. In particular, the disclosed compounds and pharmaceutical compositions may also be used in methods for prostate cancer, such as castration-resistant prostate cancer (CRPC), breast cancer, and endometrial cancer. The methods further include cotherapeutic methods for improving treatment outcomes in the context of cancers.

An overview of steroid metabolism in CRPC points to a critical role for 3β-HSD1. There are at least 3 possible pathways to dihydrotestosterone (DHT) synthesis from non-gonadal precursors 1) The canonical pathway which requires conversion from adrenal dehydroepiandrosterone (DHEA)→androstenedione (AD)→testosterone→DHT; 2) the 5α-androstanedione pathway still utilizes adrenal precursors, although it circumvents testosterone and converts androstenedione→5α-androstanedione→DHT; 3) the “backdoor” pathways may occur using de novo steroidogenesis from cholesterol and circumvent testosterone by way of 5α-reduction of progesterone or 17OH-progesterone, which then requires 5α-androstanediol as an intermediate metabolite that is then converted to DHT. Evidence exists for all 3 pathways and prior work suggests that when tumors utilize adrenal precursors, the 5α-androstanedione pathway is dominant in models as well in freshly collected metastatic CRPC tissues in which metabolism was assessed (Chang et al. P. Natl. Acad. Sci. USA 108, 13728-13733 (2011). Nonetheless, it is important to note that all pathways for the synthesis of testosterone and/or DHT require 38-HSD enzymatic activity. 3βHSD performs 2 reactions that are necessary to convert the 3β-OH, Δ⁵-structure of DHEA and cholesterol to biologically active androgens. The first is oxidation of 3β-OH to 3-keto and the second is Δ⁵→Δ⁴ isomerization (Evaul et al. Endocrinology 151, 3514-3520, (2010); Simard et al. Endocr. Rev. 26, 525-582, (2005)). Humans have 2 isoenzymes for 3β-HSD, 3β-HSD1 being the predominant peripherally expressed isoenzyme (Simard 2005; Sharifi, Endocrinology 154, 4010-4017 (2013). Together, these observations bring the central role of 3β-HSD1 in prostate cancer into focus.

Further investigation into the HSD3β1 cellular metabolic phenotype uncovered a gain-of-function missense in 3β-HSD1 that increases conversion from adrenal-derived DHEA to AD and downstream DHT synthesis in human prostate cancer cell line models (Chang et al. Cell 154, 1074-1084 (2013). A single nucleotide change converting A→C at position 1245 of HSD3B1 exchanges an asparagine (N) for a threonine (T) at 3β-HSD1 amino acid position 367. This missense is a frequent germline variant that is present at an allele frequency of approximately 25-35%-meaning at least one copy is present in about 50% of all men (Chang 2013). The variant HSD3β1 (1245C) that encodes for the 3β-HSD1 (367T) enzyme missense impedes ubiquitination and rapid proteasome-mediated degradation, thereby allowing increased steady-state 3β-HSD1 protein levels. Therefore, the HSD3β1 (1245C) allele is the adrenal-permissive allele that enables conversion from DHEA to potent downstream androgens (e.g., testosterone and DHT) that stimulate AR, whereas the HSD3β1 (1245A) allele is the adrenal-restrictive allele that impedes conversion from DHEA to potent androgens (Sabharwal et al. Endocrinology 160, 2180-2188 (2019)).

Inheritance of the adrenal-permissive HSD3β1(1245C) allele, hastening what is normally the rate-limiting step of conversion from DHEA to DHT, may allow prostate cancer to regenerate potent androgens more rapidly in men treated with ADT, thereby worsening clinical outcomes associated with CRPC. This was initially tested retrospectively in 3 cohorts (2 with biochemical recurrence after prostatectomy and 1 with metastatic CSPC) of 443 men treated with ADT. All 3 cohorts showed worse outcomes (including worse progression-free survival and overall survival) in men inheriting the adrenal permissive HSD3B1 genotypes. These findings have now been further validated now in at least 10 cohorts (Agarwal et al. JAMA Oncol. 3, 856-857 (2017); Garcia Gil et al. In Proceedings of the 4th European Conference of Oncology Pharmacy, 25-27 Oct. 2018 (Nantes, France) Abstract 107; Hearn et al. JAMA Oncol. 4, 558-562 (2018); Hearn et al. JAMA Oncol. 6 (4): e196796 (2020)).

HSD3B1 genotype analysis revealed silencing 3β-HSD1 (367T) in LNCaP cells, that have endogenous expression of HSD3β1 (1245C) which encodes for 3β-HSD1 (367T), blocks metabolic flux from [H]-DHEA (100 nM) to AD as well as further downstream conversion to 5α-androstanedione and DHT. Genetic depletion also inhibits AR-regulated gene expression (PSA, TMPRSS2) and blocks CRPC growth in LNCaP xenografts (shHSD3β1 #1, p=0.002 and shHSD3β1 #2, and 0.003). Collectively, these data credential the adrenal-permissive HSD3β1(1245C)-encoded enzyme as a pharmacologic target that genetically drives androgen synthesis and CRPC in ˜50% of all men (Chang 2013).

Several reports have documented links between 3βHSD1 and breast cancer (see, e.g., Kruse et al. JCI Insight. 6:29):e150403 (2021); Flanagan et al. Ann. Surg. Oncol. 29 (11): 7194-7201 (2022)). Furthermore, trilostane is known to inhibit 3βHSD1 and has been shown to have activity against metastatic breast cancer (Chu et al. Breast Cancer Res. Treat. 13 (2): 117-21 (1989)).

Accordingly, disclosed herein is a method of treating cancer in a subject in need thereof, comprising administering to the subject a therapeutically effective amount of a compound disclosed herein (e.g., a compound of formula (I)), or a pharmaceutically acceptable salt thereof. In some embodiments, the cancer is prostate cancer, breast cancer, or endometrial cancer.

In some embodiments, the cancer is prostate cancer. In some embodiments, the cancer is castration-resistant prostate cancer. In some embodiments, the cancer is prostate cancer, and the subject is a human having the HSD3β1 (1245C) allele. Determining the HSD3β1 genotype in a subject can be carried at as previously disclosed (Thomas et al. Urology 145, 13-21 (2020)). In some embodiments, the method comprises: identifying a human subject having prostate cancer; determining whether the subject has the HSD3β1 (1245C) allele; and, if the subject has the HSD3β1 (1245C) allele, administering to the subject a therapeutically effective amount of a compound disclosed herein (e.g., a compound of formula (I)), or a pharmaceutically acceptable salt thereof.

In some embodiments, the cancer is breast cancer. In some embodiments, the cancer is breast cancer, and the subject is a human having the HSD3β1 (1245C) allele. See, e.g., Kruse et al. JCI Insight. 6 (20): e150403 (2021). In some embodiments, the method comprises: identifying a human subject having breast cancer; determining whether the subject has the HSD3β1 (1245C) allele; and, if the subject has the HSD3β1 (1245C) allele, administering to the subject a therapeutically effective amount of a compound disclosed herein (e.g., a compound of formula (I)), or a pharmaceutically acceptable salt thereof. In some embodiments, the cancer is breast cancer that is resistant to standard hormonal therapies (e.g., aromatase inhibitors, selective estrogen receptor modulators (SERMs), estrogen receptor degraders, ovarian ablation, combinations of these and combinations with non-hormonal therapeutic agents, including CDK4/6 inhibitors).

In some embodiments, the cancer is endometrial cancer.

### Companion Diagnostic Methods

In some embodiments, the compounds provided herein are used in methods of personalized cancer therapy. In some embodiments, the compounds provided herein are used in companion diagnostic methods, which involve first determining whether a subject would be responsive to treatment with the compounds provided herein and subsequently providing/administering the compounds to the subject when a positive response is predicted. Accordingly, in some embodiments the methods provided herein comprise determining whether a subject has a cancer that would be responsive to inhibition of 3βHSD1 using a compound provided herein. A positive response to the compound (e.g., a compound of formula (I), or a pharmaceutically acceptable salt thereof) indicates that the compound has effective anti-cancer properties in the subject. In contrast, no response or a negative response to the compound indicates that the compound would likely not be effective at treating the cancer in the subject. In some embodiments, the methods provided herein comprise determining whether a subject has a cancer that would be responsive to inhibition of 3βHSD1 using a compound provided herein, and providing the compound (e.g., a compound of formula (I), or a pharmaceutically acceptable salt thereof) to the subject when a positive response to inhibition of 3βHSD1 is predicted. In some aspects, determining whether a subject has a cancer that would be responsive to inhibition of 3βHSD1 (e.g. predicting responsiveness to treatment with a compound provided herein) comprises determining whether a gain of stability mutation leading to a gain of function in 38-hydroxysteroid dehydrogenase type 1 (3βHSD1) is present in the subject. In some embodiments, the subject is a human, and the gain of stability mutation comprises a cytosine nucleotide at position 1245 of the human HSD3β1 gene which causes a threonine at position 367 of the human 3βHSD1 protein. The HSD3β1 gene (human) refers to NCBI Gene ID 3283, the sequence of which is NCBI Reference Sequence: NG_050909.1. In some embodiments, the sequence of a native human 3βHSD1 protein not having the gain of stability mutation at position 367 is MTGWSCLVTGAGGFLGQRIIRLLVKEKELKEIRVLDKAFGPELREEFSKLQNKTKLTVLE GDILDEPFLKRACQDVSVIIHTACIIDVFGVTHRESIMNVNVKGTQLLLEACVQASVPVF IYTSSIEVAGPNSYKEIIQNGHEEEPLENTWPAPYPHSKKLAEKAVLAANGWNLKNGGT LYTCALRPMYIYGEGSRFLSASINEALNNNGILSSVGKFSTVNPVYVGNVAWAHILALR ALQDPKKAPSIRGQFYYISDDTPHQSYDNLNYTLSKEFGLRLDSRWSFPLSLMYWIGFLL EIVSFLLRPIYTYRPPFNRHIVTLSNSVFTFSYKKAQRDLAYKPLYSWEEAKQKTVEWVG SLVDRHKENLKSKTQ (SEQ ID NO: 1). In some embodiments, the sequence of a human 3βHSD1 protein having a threonine at position 367 is MTGWSCLVTGAGGFLGQRIIRLLVKEKELKEIRVLDKAFGPELREEFSKLQNKTKLTVLE GDILDEPFLKRACQDVSVIIHTACIIDVFGVTHRESIMNVNVKGTQLLLEACVQASVPVF IYTSSIEVAGPNSYKEIIQNGHEEEPLENTWPAPYPHSKKLAEKAVLAANGWNLKNGGT LYTCALRPMYIYGEGSRFLSASINEALNNNGILSSVGKFSTVNPVYVGNVAWAHILALR ALQDPKKAPSIRGQFYYISDDTPHQSYDNLNYTLSKEFGLRLDSRWSFPLSLMYWIGFLL EIVSFLLRPIYTYRPPFNRHIVTLSNSVFTFSYKKAQRDLAYKPLYSWEEAKQKTVEWVG SLVDRHKETLKSKTQ (SEQ ID NO: 2). Accordingly, in some embodiments the methods comprise determining whether the subject expresses the gain of stability mutation by determining whether cells in a sample obtained from a subject express SEQ ID NO: 2 or the gene encoding SEQ ID NO: 2 (indicating that the mutation is present) or whether the cells express SEQ ID NO: 1 or the gene encoding SEQ ID NO: 1 (indicating that the gain of stability mutation is not present.)

Accordingly, in some embodiments the methods comprise determining if the HSD3β1 (1245C) gene or 3βHSD1 (367T) protein is expressed in a biological sample obtained from the subject, and predicting a positive response to the compound if the HSD3β1 (1245C) gene or 3βHSD1 (367T) protein is expressed.

In some embodiments, the method comprises obtaining a biological sample from the subject. A “biological sample,” as used herein, is meant to include any biological sample from a subject that is suitable for analysis for detection of the HSD3β1 (1245C) gene or the 3βHSD1 (367T) protein. Suitable biological samples include but are not limited to bodily fluids such as blood-related samples (e.g., whole blood, serum, plasma, and other blood-derived samples), urine, saliva, sputum, cerebral spinal fluid, bronchoalveolar lavage, and the like. Another example of a biological sample is a tissue sample. In some embodiments, the biological sample is a cancer cell or tissue including cancer cells. The HSD3β1 (1245C) gene or the 3βHSD1 (367T) protein can be assessed either quantitatively or qualitatively, and detection can be determined either in vitro or ex vivo.

The methods involve providing or obtaining a biological sample from the subject, which can be obtained by any known means including needle stick, needle biopsy, swab, and the like. In an exemplary method, the biological sample is a blood sample, which may be obtained for example by venipuncture.

A biological sample may be fresh or stored. Biological samples may be or have been stored or banked under suitable tissue storage conditions. The biological sample may be a tissue sample expressly obtained for the assays of this invention or a tissue sample obtained for another purpose which can be subsampled for the assays of this invention. Preferably, biological samples are either chilled or frozen shortly after collection if they are being stored to prevent deterioration of the sample.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC) or HPLC, or precipitation of apolipoprotein B containing proteins with dextran sulfate or other methods. Any of a number of standard aqueous buffer solutions at physiological pH, such as phosphate, Tris, or the like, can be used.

Either the variant form of the gene (HSD3β1 (1245C)) or the variant form of the protein (3βHSD1 (367T)) can be detected in the sample obtained from the subject and used in the determination of whether the subject would have a positive response to a compound provided herein. The gene or protein can be detected or measured by an analytic device such as a kit or a conventional laboratory apparatus, which can be either portable or stationary. In some embodiments, the levels of variant gene or protein may be compared to the level of corresponding internal standards in the sample or samples when carrying out the analysis to quantify the amount of the gene or protein being detected.

The HSD3β1 (1245C) gene or the 3βHSD1 (367T) protein are typically detected in a biological sample which has been obtained from the subject. However, in some embodiments, noninvasive imaging modalities for detecting this mutation are used. For example, administration of 18F-DHEA with PET imaging may be able to detect tumors that harbor the mutant enzyme because these tumors are anticipated to have great flux from DHEA to downstream androgen metabolites.

In some embodiments, the presence of the 3βHSD1 (367T) protein is determined. The presence and/or amount of the 3βHSD1 (367T) protein in a biological sample can be determined using polyclonal or monoclonal antibodies that are immunoreactive with the 3βHSD1 (367T) protein variant. Use of antibodies comprises contacting a sample taken from the individual with one or more of the antibodies; and assaying for the formation of a complex between the antibody and a protein or peptide in the sample. For ease of detection, the antibody can be attached to a substrate such as a column, plastic dish, matrix, or membrane, preferably nitrocellulose. The sample may be untreated, subjected to precipitation, fractionation, separation, or purification before combining with the antibody. Interactions between antibodies in the sample and the 3βHSD1 (367T) protein are detected by radiometric, colorimetric, or fluorometric means, size-separation, or precipitation. Preferably, detection of the antibody-protein or peptide complex is by addition of a secondary antibody that is coupled to a detectable tag, such as for example, an enzyme, fluorophore, or chromophore. Formation of the complex is indicative of the presence of the 3βHSD1 (367T) protein in the sample.

Antibodies immunospecific for 3βHSD1 (367T) may be made and labeled using standard procedures and then employed in immunoassays to detect the presence of 3βHSD1 (367T) in a sample. Suitable immunoassays include, by way of example, immunoprecipitation, particle immunoassay, immunonephelometry, radioimmunoassay (RIA), enzyme immunoassay (EIA) including enzyme-linked immunosorbent assay (ELISA), sandwich, direct, indirect, or competitive ELISA assays, enzyme-linked immunospot assays (ELISPOT), fluorescent immunoassay (FIA), chemiluminescent immunoassay, flow cytometry assays, immunohistochemistry, Western blot, and protein-chip assays using for example antibodies, antibody fragments, receptors, ligands, or other agents binding the target analyte. Polyclonal or monoclonal antibodies raised against 3βHSD1 (367T) are produced according to established procedures. Generally, for the preparation of polyclonal antibodies, a protein or peptide fragment thereof is used as an initial step to immunize a host animal. A general review of immunoassays is available in Methods in Cell Biology v. 37: Antibodies in Cell Biology, Asai, ed. Academic Press, Inc. New York (1993), and Basic and Clinical Immunology 7th Ed., Stites & Ten, eds. (1991).

In some embodiments, the 3βHSD1 (367T) protein is detected using a method other than an immunoassay. For example, the 3βHSD1 (367T) protein can be detected using matrix-assisted laser desorption-ionization time-of-flight mass spectrometry (MALDI-TOF). The 3βHSD1 (367T) protein can also be detected by purifying the 3βHSD1 protein and determining its sequence using peptide sequencing methods. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified and/or quantified using chromatographic and electrophoretic techniques to achieve partial or complete purification (or purification to homogeneity). Analytical methods particularly suited to the preparation of a pure peptide are immunohistochemistry, ion-exchange chromatography, exclusion chromatography; polyacrylamide gel electrophoresis; isoelectric focusing. A particularly efficient method of purifying peptides is fast protein liquid chromatography or even HPLC. Likewise, a variety of methods of protein sequencing are known to those skilled in the art. For example, the sequence may be identified using mass spectrometry or the Edman degradation reaction.

The 3βHSD1 (367T) protein can also be detected based on its differing characteristics relative to the wild-type version of the protein. The inventors have demonstrated that the 3βHSD1 (367T) protein is more resistant to ubiquitination and degradation than the 3βHSD1 (367N) protein. Accordingly, the presence of the 3βHSD1 (367T) protein can be detected using a ubiquitination and/or degradation assay. Likewise, the inventors have shown that the autocrine mobility factor receptor (AMFR), which is involved in protein degradation, shows a lower affinity for the 36HSD1 (367T) protein than the 3βHSD1 (367N) protein. Accordingly, the presence of the 3βHSD1 (367T) protein can also be determined by evaluating the affinity of the 3βHSD1 for AMFR. Some tumors may also have somatic loss of expression of AMFR, or other methods of wild-type enzyme (3βHSD1 (367N)) stabilization that might serve the tumor as a compensatory mechanism of protein stabilization, leading to increased DHT synthesis and treatment-resistance.

In some embodiments, the presence of the 3βHSD1 (367T) can be detected indirectly by observed the serum steroid profile. Assessment of the serum steroid profile could correlate with HSD3β1 genotype. For example, the mutant enzyme may be associated with an increase in the ratio of enzyme product vs. precursor (i.e., androstenedione/DHEA and progesterone/pregnenolone).

In some embodiments, the presence of the HSD3β1 (1245C) allele is determined. The presence and/or the level of the HSD3β1 (1245C) allele can be determined by any now known or hereafter developed assay or method of detecting and/or determining expression level, for example, quantitative RT-PCR, Northern blot, real-time PCR, PCR, allele-specific PCR, pyrosequencing, SNP Chip technology, sequencing, or restriction fragment length polymorphism (RFLP).

Many methods for determining a nucleotide sequence involve PCR. As used herein, the term “polymerase chain reaction” (PCR) refers to the methods of U.S. Pat. Nos. 4,683,195, 4,683,202, and 4,965,188, all of which are hereby incorporated by reference, directed to methods for increasing the concentration of a segment of a target sequence in a mixture of genomic DNA without cloning or purification. As used herein, the terms “PCR product” and “amplification product” refer to the resultant mixture of compounds after two or more cycles of the PCR steps of denaturation, annealing and extension are complete. These terms encompass the case where there has been amplification of one or more segments of one or more target sequences. Accordingly, in some embodiments, the detecting the presence and/or level of the HSD3β1 (1245C) allele comprises extending a primer that hybridizes to a sequence adjacent to the polymorphic nucleotide. In some embodiments, the determining the presence and/or level of the HSD3β1 (1245C) allele comprises hybridizing a probe to a region that includes the polymorphic nucleotide.

In some embodiments, hybridization with complementary sequences may be used to detect the presence of the HSD3β1 (1245C) allele based on the different characteristics of sequences that have a complete or incomplete sequence match. For example, as described herein, an asymmetric PCR assay can be used in which fluorescence melting reveals two distinct melting temperatures of the probe/target duplex that are specific for the amplified allele. This assay can be used to detect the HSD3β1 (1245C) allele in the germline or in somatic cells.

In some embodiments, the presence of the HSD3β1 (1245C) allele is detected by sequencing, including next-generation sequencing (NGS) techniques. The term “sequencing” encompasses a variety of suitable sequencing techniques that can be used to determine the quantity and type of nucleic acid present in a sample, which is indicative of whether the HSD3β1 (1245C) allele is present in the subject. In some embodiments, sequencing involves isolating RNA from a sample, generating a cDNA library from the RNA, and then sequencing the cDNA. In some embodiments, sequencing involves massively parallel sequencing. In some embodiments, sequencing involves sequencing nucleic acid molecules directly, such as through massively-parallel direct RNA-seq. Suitable sequencing methods include sequencing-by-synthesis methods (SBS), reversible terminator sequencing, sequencing by ligation, sequencing by hybridization and ligation, sequencing by hybridization and synthesis, nanopore sequencing, nanoball sequencing, and the like.

Once the presence and/or levels of the variant form of either the gene (HSD3β1 (1245C)) or the variant form of the protein (3βHSD1 (367T)) have been determined, they can be displayed in a variety of ways. For example, the levels can be displayed graphically on a display as numeric values or proportional bars (i.e., a bar graph) or any other display method known to those skilled in the art. The graphic display can provide a visual representation of the amount of the variant gene or protein in the biological sample being evaluated.

### Additional Methods of Use

Also disclosed herein is a method of inhibiting cancer cell proliferation, comprising contacting cancer cells with a compound disclosed herein (e.g., a compound of formula (I)), or a pharmaceutically acceptable salt thereof, in an amount effective to inhibit the cancer cell proliferation. In some embodiments, the cancer cells are prostate cancer cells. In some embodiments, the cancer cells are castration-resistant prostate cancer cells. In some embodiments, the cancer cells are breast cancer cells. In some embodiments, the cancer cells are endometrial cancer cells. In some embodiments, the cancer cells are predicted to have a positive response to the compound, as described above.

Also disclosed herein is a method of inhibiting the activity of 3β-hydroxysteroid dehydrogenase in a sample, comprising contacting the sample with a compound disclosed herein (e.g., a compound of formula (I)), or a pharmaceutically acceptable salt thereof, in an amount effective to inhibit the activity of 36-hydroxysteroid dehydrogenase.

### Routes of Administration and Combination Therapies

In the methods of treatment described herein, a compound or pharmaceutical composition may be administered to the subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; or by implant of a depot, for example, subcutaneously or intramuscularly. In some embodiments, the administration comprises oral administration. Additional modes of administration may include adding the compound and/or a composition comprising the compound to a food or beverage, including a water supply for an animal, to supply the compound as part of the animal's diet.

It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments of the present disclosure. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. In general, a suitable dose of the compound is in the range of about 100 μg to about 250 mg per kilogram body weight of the subject per day.

The compound or composition may be administered once, on a continuous basis (e.g. by an intravenous drip), or on a periodic/intermittent basis, including about once per hour, about once per two hours, about once per four hours, about once per eight hours, about once per twelve hours, about once per day, about once per two days, about once per three days, about twice per week, about once per week, and about once per month. The composition may be administered until a desired reduction of symptoms is achieved.

A compound described herein may be used in combination with other known therapies. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A compound or composition described herein and the at least one additional therapeutic agent can be administered simultaneously, in the same or in separate compositions, or sequentially. For sequential administration, the compound described herein can be administered first, and the additional agent can be administered subsequently, or the order of administration can be reversed.

In some embodiments, a compound described herein is administered in combination with other therapeutic treatment modalities, including surgery, chemotherapy, radiation therapy, hormone therapy, immunotherapy, cryotherapy, and thermotherapy. Such combination therapies may advantageously utilize lower dosages of the administered agent and/or other chemotherapeutic agent, thus avoiding possible toxicities or complications associated with the various therapies. The phrase “radiation” includes, but is not limited to, external-beam therapy which involves three-dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy. In some embodiments, the second therapy includes immunotherapy. Immunotherapies include chimeric antigen receptor (CAR) T-cell or T-cell transfer therapies, cytokine therapy, immunomodulators, cancer vaccines, or administration of antibodies (e.g., monoclonal antibodies).

In some embodiments, the compound described herein is administered with at least one additional therapeutic agent, such as a chemotherapeutic agent. In certain embodiments, the compound described herein is administered in combination with one or more additional chemotherapeutic agents. The chemotherapeutic agent may be a chemotherapeutic agent identified on the “A to Z List of Cancer Drugs” published by the National Cancer Institute. In some embodiments, the chemotherapeutic agent is selected from abiraterone, apalutamide, bicalutamide, cabazitaxel, capecitabine, cyclophosphamide, darolutamide, degarelix, docetaxel, dutasteride, enzalutamide, estradiol, estramustine, finasteride, flutamide, goserelin, histrelin, leuprolide, mitoxantrone, nilutamide, olaparib, radium-223, rucaparib, sipuleucel-T, and triptorelin, or any combination thereof. In some embodiments, the chemotherapeutic agent is selected from abemaciclib, ado-trastuzumab emtansine, alpelisib, anastrozole, atezolizumab, capecitabine, carboplatin, cisplatin, cyclophosphamide, docetaxel, doxorubicin, epirubicin, eribulin, everolimus, exemestane, fam-trastuzumab deruxtecan, 5-fluorouracil, fulvestrant, gemcitabine, goserelin, ixabepilone, lapatinib, letrozole, margetuximab-cmkb, megestrol, methotrexate, neratinib, olaparib, paclitaxel, palbociclib, pamidronate, pembrolizumab, pertuzumab, raloxifene, ribociclib, sacituzumab govitecan-hziy, talazoparib, tamoxifen, thiotepa, toremifene, trastuzumab, tucatinib, vinblastine, and vinorelbine, or any combination thereof. In some embodiments, the chemotherapeutic agent is selected from carboplatin, cisplatin, docetaxel, dostarlimab-gxly, doxoribucin, ifosfamide, lenvatinib, megestrol, paclitaxel, and pembrolizumab, trastuzumab, or any combination thereof.

### Kits

The present disclosure also provides kits for guiding the treatment of cancer in a subject using a compound described herein. For example, the kits can be used in a method for personalized medicine or a companion diagnostic method involving assessing 3βHSD1 (367T) or HSD3β1 (1245C) in a sample obtained from a subject having or at risk of having cancer, and providing to the subject a compound described herein when a positive response to the compound is predicted based upon expression of 3βHSD1 (367T) or HSD3β1 (1245C) in the sample.

The kits include one or more primers or probes capable of detecting 3βHSD1 (367T) or HSD3β1 (1245C), and a package for holding the primers or probes. A kit generally includes a package with one or more containers holding the reagents, as one or more separate compositions or, optionally, as an admixture where the compatibility of the reagents will allow. The kits may further include enzymes (e.g., polymerases), buffers, labeling agents, nucleotides, controls, and any other materials necessary for carrying out the detection of 3βHSD1 (367T) or HSD3β1 (1245C). Kits can also include a tool for obtaining a sample from a subject, such as a punch tool to obtain a punch-biopsy or needle biopsy.

In some embodiments, the kit includes a primer capable of detecting HSD3β1 (1245C). For example, the kits may include suitably sized oligonucleotide primers that amplify a region of HSD3β1 including the A→C conversion. For example, in some embodiments, a region of from HSD3β1 including the A→C conversion and including from about 10 nucleotides to about 100 nucleotides can be used. Specific suitable primers are describing the Example, herein. The primers may be labeled.

In another aspect, kits for guiding treatment are provided that include an array and/or microarray, oligonucleotide primes that amplify from about nucleotide 1200 to about nucleotide 1300 portion of HSD3β1 and instructions for use. Alternately or in addition, primers may be provided that amplify from about nucleotide 1210 to about nucleotide 1280 of HSD3β1, from about nucleotide 1220 to about nucleotide 1270 of HSD3β1, from about nucleotide 1230 to about nucleotide 1260 of HSD3β1, from about nucleotide 1235 to about nucleotide 1250 of HSD3β1, or other portion that one of skill in the art would determine necessary or adequate to amplify and detect the presence of HSD3β1 (1245C) using PCR or other sequencing technology known to those skilled in the art.

In some embodiments, the kit includes a probe capable of detecting 3βHSD1 (367T). A preferred type of probe is an antibody capable of specifically binding to 3βHSD1 (367T). Examples of antibodies that can be used in the present disclosure include, but are not limited to, polyclonal antibodies, monoclonal antibodies, chimeric antibodies, human antibodies, humanized antibodies, recombinant antibodies, single-chain Fvs (“scFv”), an affinity maturated antibody, single chain antibodies, single domain antibodies, F(ab) fragments, F(ab′) fragments, disulfide-linked Fvs (“sdFv”), and antiidiotypic (“anti-Id”) antibodies and functionally active epitope-binding fragments of any of the above. As used herein, the term “specifically binding” refers to the interaction of the antibody with a second chemical species, wherein the interaction is dependent upon the presence of a particular structure (e.g., an antigenic determinant or epitope) on the chemical species; for example, an antibody recognizes and binds to a specific protein structure rather than to proteins generally.

The kits may also include a solid phase, to which the antibodies functioning as capture antibodies and/or detection antibodies in a sandwich immunoassay format are bound. The solid phase may be a material such as a magnetic particle, a bead, a test tube, a microtiter plate, a cuvette, a membrane, a scaffolding molecule, a quartz crystal, a film, a filter paper, a disc or a chip. The kit may also include a detectable label that can be or is conjugated to an antibody, such as an antibody functioning as a detection antibody. The detectable label can for example be a direct label, which may be an enzyme, oligonucleotide, nanoparticle chemiluminophore, fluorophore, fluorescence quencher, chemiluminescence quencher, or biotin. Test kits may optionally include any additional reagents needed for detecting the label.

The kit can also include instructions for using the kit to carry out a method of guiding treatment of steroid-dependent disease in a subject. In some embodiments, the steroid-dependent disease is a steroid-dependent cancer, such as prostate cancer. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The following examples further illustrate aspects of the disclosure but, of course, should not be construed as in any way limiting its scope.

### EXAMPLES

The following abbreviations are used in the Examples: “DCM” means dichloromethane, “DMAP” means 4-dimethylaminopyridine, “DMF” means N,N-dimethylformamide, “DMSO” means dimethylsulfoxide, “DTBAD” means di-tert-butyl azodicarboxylate, “EtOAc” means ethyl acetate, “HPLC” means high performance liquid chromatography, “LCMS” or “LC-MS” means liquid chromatography/mass spectrometry, “LiHMDS” means lithium bis(trimethylsilyl)amide, “KO^{t}Bu” means potassium tert-butoxide, “MeI” means methyl iodide, “MeOH” means methanol, “[M+H]⁺” means the protonated mass of the free base of the compound, “mp” means melting point, “NMR” means nuclear magnetic resonance, “Pd(amphos)Cl₂” means bis(di-tert-butyl(4-dimethylaminophenyl)phosphine)dichloropalladium(II), “R” means retention time (in minutes), “THF” means tetrahydrofuran, “tR” means retention time, and “Xphos palladacycle G2” means chloro(2-dicyclohexylphosphino-2′,4′,6′-triisopropyl-1,1′-biphenyl)[2-(2′-amino-1,1′-biphenyl)]palladium(II).

### Experimental

Hydrogenation reactions were performed using an atmospheric balloon or using a Parr hydrogenation shaker apparatus. Analytical thin layer chromatography (TLC) was performed on Keislegel 60 F 250 micron precoated glass plates and eluted using reagent grade solvents. TLC plates were visualized with UV light and iodine.

Normal phase flash silica gel-based column chromatography was performed using ready-to-connect cartridges on a Teledyne Isco Combiflash® NextGen 300 system. Ready connect cartridges are Teledyne RediSep® Rf preparative silica columns with an average particle size of 35 to 70 microns.

High resolution mass spectra were recorded on an Agilent 1290 Infinity II Series 6230B TOF LC/MS. Detection methods are diode array (DAD) at 210, 254 nM and positive/negative electrospray ionization (ESI), mass range capable of 25-20,000 m/z. The MS detector was configured to mass range 100 to 1700 m/z, with nitrogen used as the nebulizer gas. High-resolution acquisition rate mass spectra (MS mode) were acquired in electrospray mode by scanning at a rate up to 40 spectra per second. The system maintains a 2 ppm mass accuracy/stability within 2° C. drift per hour. Data acquisition was performed with MassHunter Walkup software. Unless stated, all methods use an Agilent InfinityLab Poroshell 120 EC-C18 column, dimensions 4.6×50 mm, 2.7 μm, fitted with Poroshell 120 EC-C18, 2.1 mm, 1.9 μm guard. Mobile phase A was 0.1% TFA in H₂O, mobile phase B was 0.1% TFA in CH₃CN.

For LC-MS-characterization of the compounds of the present invention, the following methods were used.
Method 1: Reverse phase HPLC was carried out with a flow rate of 0.4 mL/min, at 55° C., using positive ESI mode. Injection volume 1 μL. The gradient conditions used are 5% mobile phase B for 0.2 min., then a gradient of 5-95% mobile phase B over 2.0 min, then hold at 95% mobile phase B for 0.45 min, returning to initial conditions at 2.65 min.
Method 2: Reversed phase HPLC was carried out with a flow rate of 0.4 mL/min, at 55° C., using positive ESI mode. Injection volume 1 μL. The gradient conditions used are 40% mobile phase B for 0.2 min., then a gradient of 40-95% mobile phase B over 2.5 min, then hold at 95% mobile phase B for 0.5 min, returning to initial conditions at 3.2 min.
Method 3: Reverse phase HPLC was carried out with a flow rate of 0.4 mL/min, at 55° C., using negative ESI mode. Injection volume 1 μL. The gradient conditions used are 5% mobile phase B for 0.2 min., then a gradient of 5-95% mobile phase B over 2.0 min, then hold at 95% mobile phase B for 0.45 min, returning to initial conditions at 2.65 min.

Preparative RP-HPLC purification was performed on either a Gilson GX-271 preparative liquid handler, or Teledyne ACCQ-Prep purification system. Both instruments use UV-based peak detection and Phenomenex Kinetex C18 columns with an acetonitrile-water (0.1% TFA) custom gradient. Compounds obtained initially as a TFA salt after purification were obtained as free base either by dissolution in EtOAc and washed with sat. aq. K₂CO₃, or by elution through a Biotage ISOLUTE® SCX-II cartridge, loading and washing with MeOH and then eluting with 2N NH₃ in MeOH.

Chiral purification of racemic mixtures was readily accomplished using a supercritical fluid chromatography (SFC) instrument from using a Lab Hybrid 10-100 supercritical CO₂ chromatography system for PIC Instrument Solutions. Chiral analytical and semi-prep SFC purification columns were from Chiral Technologies.

¹H NMR spectra were recorded on a Bruker AVANCE NEO NanoBay 400 spectrometer with standard pulse sequences, operating at 400 MHz. Acquisition and processing was performed with TopSpin4 software. Chemical shifts (8) are reported in parts per million (ppm) downfield from tetramethylsilane (TMS), which was used as internal standard. Coupling constants (J-values) are reported in Hz. The following abbreviations (or a combination, thereof) are used to describe splitting patterns: s, singlet; d, doublet; t, triplet; q, quartet; pent, pentet; m, multiplet; br, broad.

### General Synthetic Procedures

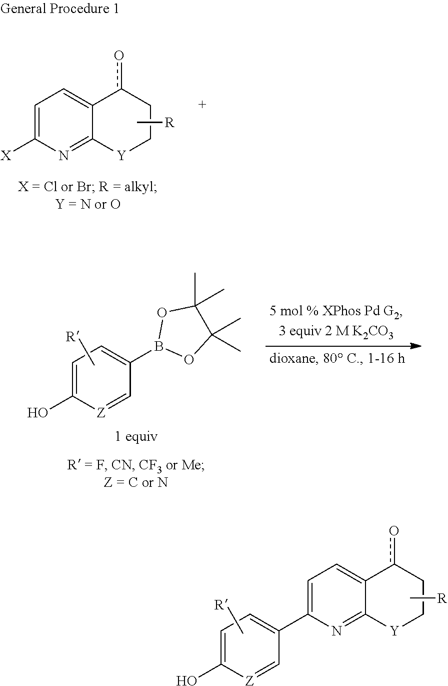

A mixture of arylboronic acid pinacol ester (1 equiv), aryl halide (1 equiv) and 2M aq. Potassium Carbonate (3 equiv) in Dioxane (0.25 M) was degassed for 10 mins before adding XPhos palladacycle G2 (5 mol %). The reaction mixture was stirred at 80° C. for 1-16 h. After completion the mixture was diluted with EtOAc, poured into the separating funnel containing 1M HCl, organic layer was separated, dried over Na₂SO₄, concentrated in vacuo and purified by ISCO flash chromatography using EtOAc in hexanes to get final product.

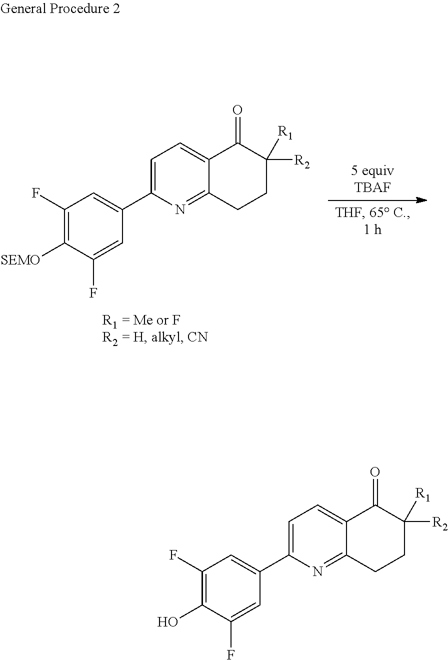

To a SEM protected phenol in THF (0.1 M) was added 1M tetra-n-butylammonium fluoride in THF (5 equiv) and the reaction mixture was stirred at 65° C. for 1 h. The reaction mixture was quenched with aq. sat. NH₄Cl, extracted with EtOAc, organic layer was separated, dried over Na₂SO₄, concentrated in vacuo and purified by ISCO flash chromatography using EtOAc in hexanes to get final product.

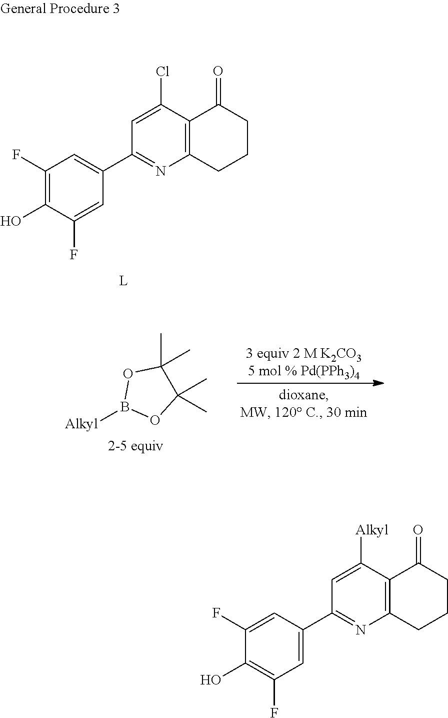

A mixture of intermediate L, alkylboronic acid pinacol ester (2-5 equiv) and 2M aq. Potassium Carbonate (3 equiv) in Dioxane (0.1 M) was degassed for 10 mins before adding Pd(PPh₃)₄ (5 mol %). The reaction mixture was stirred in microwave at 120° C. for 30 min. After completion the mixture was diluted with EtOAc, poured into the separating funnel containing 1M HCl, organic layer was separated, dried over Na₂SO₄, concentrated in vacuo and purified by ISCO flash chromatography using EtOAc in hexanes to get final product.

### Preparation of Intermediates

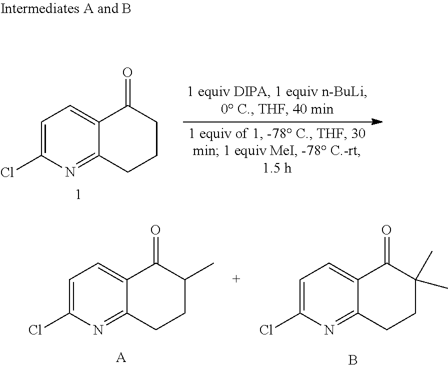

Diisopropylamine (1.16 mL, 8.26 mmol) and n-BuLi (3.3 mL, 8.26 mmol) in 12 mL THF was stirred at 0° C. for 40 min. To this solution was added 2-Chloro-7,8-dihydroquinolin-5(6H)-one (1.5 g, 8.26 mmol) dissolved in 12 mL THF at −78° C. After stirring the reaction mixture for 30 min at the same temperature MeI (0.51 mL, 8.26 mmol) was added followed by stirring at room temperature for 1.5 h. The reaction mixture was quenched with sat. aq. NH₄Cl, extracted with EtOAc, dried over Na₂SO₄ and concentrated in vacuo. Purification by ISCO flash chromatography (40 g, 10% EtOAc in hexanes) gave a white solid for 2-chloro-6-methyl-7,8-dihydro-6H-quinolin-5-one A (250 mg, 1.2778 mmol, 15% yield). Purity ≥95% by LCMS (210, 254 nm); tR=1.07 m/z=196.05 [M+H]⁺ and an off-white solid for 2-chloro-6,6-dimethyl-7,8-dihydroquinolin-5-one B (330 mg, 1.5739 mmol, 19% yield). Purity ≥95% by LCMS (210, 254 nm); tR=1.32 m/z=210.06 [M+H]⁺

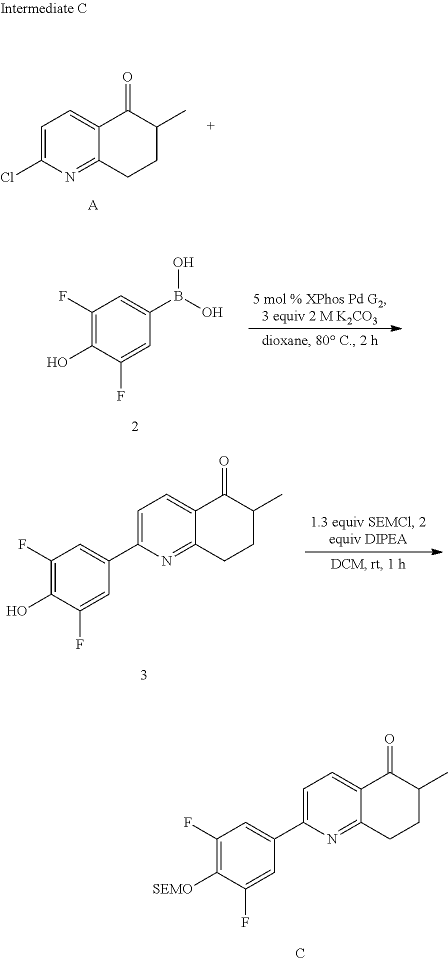

A mixture of 3,5-difluoor-4-hydroxyphenylboronic acid (235.64 mg, 1.35 mmol), 2-chloro-6-methyl-7,8-dihydro-6H-quinolin-5-one (241 mg, 1.23 mmol) and 2M aq Potassium Carbonate (1.85 mL, 3.7 mmol) in Dioxane (6 mL) was degassed for 10 mins before adding XPhos palladacycle G2 (48.46 mg, 0.06 mmol). The reaction mixture was stirred at 80° C. for 2 h. After completion the mixture was diluted with EtOAc, poured into the separating funnel containing 1M HCl, organic layer was separated, dried over Na₂SO₄ and concentrated in vacuo. Purification by ISCO flash chromatography (12 g, 30% EtOAc in hexanes) gave a light yellow powder of 2-(3,5-difluoro-4-hydroxy-phenyl)-6-methyl-7,8-dihydro-6H-quinolin-5-one 3 (212 mg, 0.7329 mmol, 59% yield). Purity ≥95% by LCMS (210, 254 nm); tR=1.15 m/z=290.08 [M+H]⁺

To 2-(3,5-difluoro-4-hydroxy-phenyl)-6-methyl-7,8-dihydro-6H-quinolin-5-one (212 mg, 0.73 mmol) in DCM (6 mL) was added SEM-Cl (0.17 mL, 0.95 mmol) and Diisopropylethylamine (0.26 mL, 1.47 mmol) at RT. The reaction mixture was stirred at RT for 1 h. After completion the reaction mixture was quenched with water, extracted with DCM, organic layer was separated, dried over Na₂SO₄ and concentrated in vacuo. Purification by ISCO flash chromatography (12 g, 15% EtOAc in hexanes) gave a thick oily liquid of 2-[3,5-difluoro-4-(2-trimethylsilylethoxymethoxy)phenyl]-6-methyl-7,8-dihydro-6H-quinolin-5-one C (250 mg, 0.5959 mmol, 81% yield). Purity ≥95% by LCMS (210, 254 nm); tR=2.88 m/z=420.17 [M+H]⁺

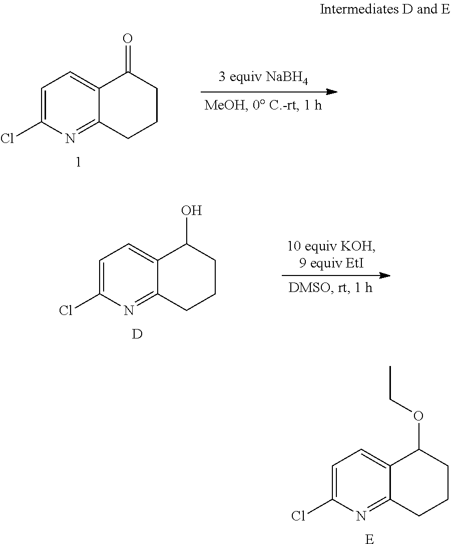

To 2-chloro-7,8-dihydroquinolin-5(6H)-one (504 mg, 2.78 mmol) in methanol (10 mL) was added sodium borohydride (314.94 mg, 8.33 mmol) at 0° C. and stirred for 1 h at room temperature. The reaction mixture was concentrated, diluted with EtOAc and water, aqueous layer was extracted with EtOAc, dried over Na₂SO₄ and concentrated in vacuo. Purification by ISCO flash chromatography (12 g, 70% EtOAc in hexanes) gave a white solid of 2-chloro-5,6,7,8-tetrahydroquinolin-5-ol D (500 mg, 2.7229 mmol, 98% yield). Purity ≥95% by LCMS (210, 254 nm); tR=1.46 m/z=184.04 [M+H]⁺

To 2-chloro-5,6,7,8-tetrahydroquinolin-5-ol (40 mg, 0.22 mmol) in DMSO (1.5 mL) was added KOH (122.2 mg, 2.18 mmol) followed by ethyl iodide (0.16 mL, 1.96 mmol) at room temperature and stirred for 1 h. Water was added to the reaction mixture and extracted with EtOAc, the organic layer was washed with brine, dried over Na₂SO₄ and concentrated. Purification by ISCO flash chromatography (12 g, 50% EtOAc in hexanes) gave a thick colorless liquid of 2-chloro-5-ethoxy-5,6,7,8-tetrahydroquinoline E (17 mg, 0.0803 mmol, 36% yield). Purity ≥95% by LCMS (210, 254 nm); tR=1.39 min, m/z=212.07 [M+H]⁺

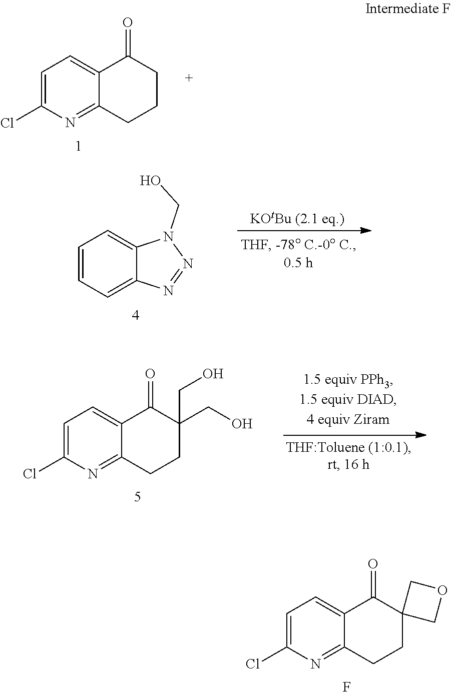

A solution of potassium tert-butoxide (2.89 mL, 2.89 mmol) was added to a suspension of 2-Chloro-7,8-dihydroquinolin-5(6H)-one (250 mg, 1.38 mmol) and 1H-benzotriazole-1-methanol (431.29 mg, 2.89 mmol) in THF (2.5 mL) over 10 minutes in a dry ice-acetone bath at −78° C. The mixture was stirred for 0.5 hours in an ice bath, and then diluted with EtOAc. After stirring for another 30 min, the mixture was filtered off. The filtrate was washed with 0.2 M aqueous NaOH (two times), water and brine, dried over Na₂SO₄ and concentrated in vacuo. Purification by ISCO flash chromatography (12 g, 30% EtOAc in hexanes) gave a colorless liquid of 2-chloro-6,6-bis(hydroxymethyl)-7,8-dihydroquinolin-5-one 5 (253 mg, 1.0469 mmol, 76% yield). Purity 295% by LCMS (210, 254 nm); tR=1.35 m/z=242.98 [M+H]⁺

2-Chloro-6,6-bis(hydroxymethyl)-7,8-dihydroquinolin-5-one (80 mg, 0.33 mmol), zinc bis(dimethyldithiocarbamate) (404.94 mg, 1.32 mmol) and triphenylphosphine (130.24 mg, 0.5 mmol) in THF (4 mL) was added diisopropyl azodicarboxylate (0.1 mL, 0.5 mmol) dissolved in Toluene (0.4 mL) in an ice-water bath. The mixture was stirred overnight at room temperature. The mixture was diluted with toluene (4 mL), and the mixture was filtered off. The filtrate was washed with 1 M aqueous NaOH (three times), water and brine, dried over Na₂SO₄, filtered and concentrated in vacuo. Purification by ISCO flash chromatography (12 g, 40% EtOAc in hexanes) gave a white solid of 2-chlorospiro[7,8-dihydroquinoline-6,3′-oxetane]-5-one F (66 mg, 0.2951 mmol, 89% yield). Purity ≥95% by LCMS (210, 254 nm); tR=1.65, m/z=224.04 [M+H]⁺

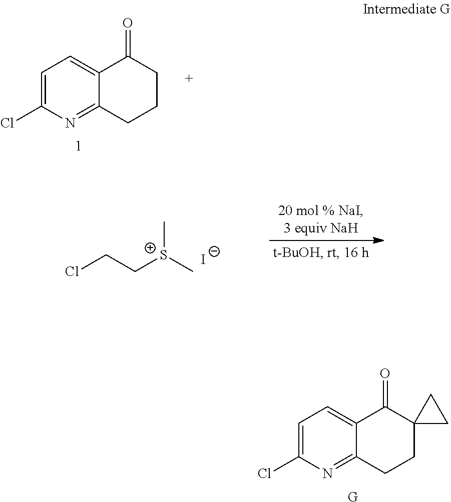

To 2-chloro-7,8-dihydroquinolin-5(6H)-one (151.03 mg, 0.83 mmol) in tert-Butanol (2 mL) was added sodium iodide (23.74 mg, 0.16 mmol) and sodium hydride (63.36 mg, 1.58 mmol) slowly at room temperature followed by stirring for 20 min. 2-chloroethyl(dimethyl)sulfonium;iodide (200 mg, 0.79 mmol) was added to the reaction mixture followed by stirring for 48 h at room temperature. The reaction was quenched with water, extracted with EtOAc, organic layer was separated, dried over Na₂SO₄ and concentrated in vacuo. Purification by ISCO flash chromatography (12 g, 15% EtOAc in hexanes) gave a white solid of 2-chlorospiro[7,8-dihydroquinoline-6,1′-cyclopropane]-5-one G (68 mg, 0.3275 mmol, 41% yield). Purity ≥95% by LCMS (210, 254 nm); tR=1.07 m/z=208.05 [M+H]⁺

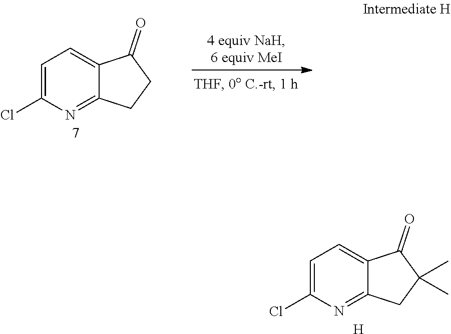

To 2-chloro-5H,6H,7H-cyclopenta[b]pyridin-5-one (63 mg, 0.38 mmol) in THF (1.5 mL) was added sodium hydride (60.15 mg, 1.5 mmol) at 0° C. and stirred for 10 min. Slowly add MeI (0.14 mL, 2.26 mmol) at 0° C., the reaction mixture was stirred at room temperature for 1 h. The reaction was diluted with EtOAc and water, extracted with EtOAc, dried over Na₂SO₄ and concentrated in vacuo. Purification by ISCO flash chromatography (12 g, 10% EtOAc in hexanes) gave a white solid of 2-chloro-6,6-dimethyl-7H-cyclopenta[b]pyridin-5-one H (42 mg, 0.2147 mmol, 57% yield). Purity ≥95% by LCMS (210, 254 nm); tR=1.04 m/z=196.05 [M+H]⁺

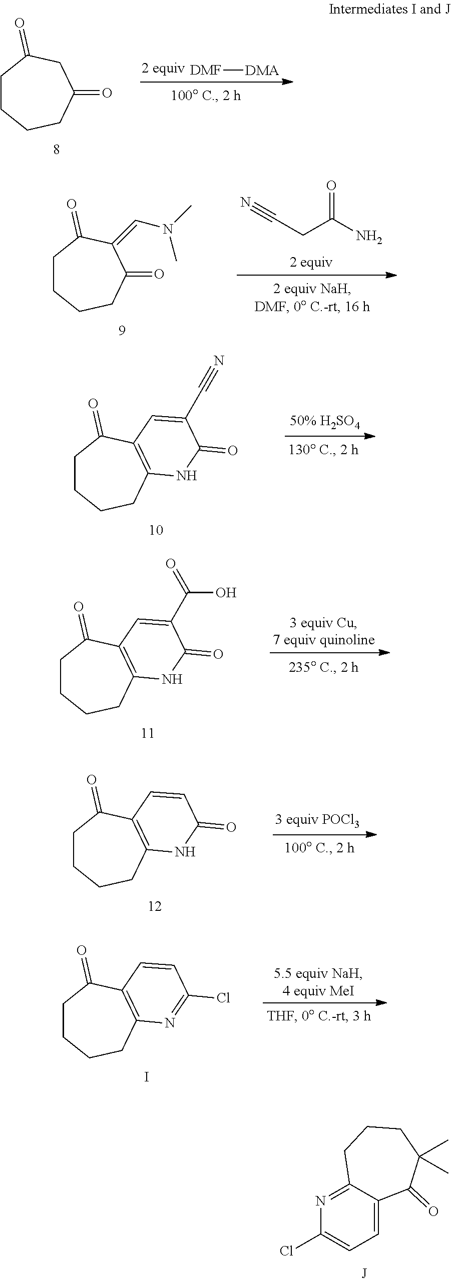

Cycloheptane-1,3-dione (3.88 mL, 33.85 mmol) was taken in N,N-Dimethylformamide dimethyl acetal (8.96 mL, 67.7 mmol) followed by stirring at 100° C. for 2 h. The reaction mixture was then concentrated to yield a black oil of 2-(dimethylaminomethylene)cycloheptane-1,3-dione 9 (6.1 g, 33.659 mmol, crude). LCMS (210, 254 nm); tR=1.18 min, m/z=182.11 [M+H]⁺

To a solution of 2-cyanoacetamide (5.66 g, 67.32 mmol) in DMF (20 mL) was added sodium hydride (1.62 g, 67.32 mmol) slowly at 0° C. After stirring at 0° C. for 30 min, a solution of 2-(dimethylaminomethylene)cycloheptane-1,3-dione (6.1 g, 33.66 mmol) in DMF (30 mL) was added followed by stirring at room temperature for 16 h. DMF was removed under reduced pressure. The residue was dissolved in water, washed with ethyl acetate three times, and neutralized with 1.0 N aqueous HCl to pH 2-3. The black-brownish precipitate was collected by filtration and dried in vacuo. The filtrate was concentrated to minimum amount of water, ice was added to obtain some more precipitate which was collected by filtration and dried in vacuo. The combined black-brownish solids gave 2,5-dioxo-6,7,8,9-tetrahydro-1H-cyclohepta[b]pyridine-3-carbonitrile 10 (4.4 g, 21.76 mmol, 64% yield for 2 steps). Purity ≥95% by LCMS (210, 254 nm); tR=1.32 min, m/z=203.08 [M+H]⁺

2,5-dioxo-6,7,8,9-tetrahydro-1H-cyclohepta[b]pyridine-3-carbonitrile (4.4 g, 21.76 mmol) was dissolved in 35 mL of 50% H₂SO₄, and stirred at 130° C. for 2 h. The reaction mixture was poured onto ice and the resultant black-brown precipitate was collected by filtration and dried in vacuo to get 2,5-dioxo-6,7,8,9-tetrahydro-1H-cyclohepta[b]pyridine-3-carboxylic acid 11 (3.6 g, 16.274 mmol, 74% yield). Purity ≥95% by LCMS (210, 254 nm); tR=1.29 min, m/z=222.05 [M+H]⁺

Copper (1.72 g, 27.12 mmol), 2,5-dioxo-6,7,8,9-tetrahydro-1H-cyclohepta[b]pyridine-3-carboxylic acid (2 g, 9.04 mmol) and quinoline (7.5 mL, 63.29 mmol) were placed in a reaction vial. The vial was then heated at 235° C. for 2 h. The reaction mixture was then diluted with chloroform, washed three times with 1N HCl (100 mL) to remove most of the quinoline, dried over Na₂SO₄, and concentrated in vacuo to get a brown solid of 6,7,8,9-tetrahydro-1H-cyclohepta[b]pyridine-2,5-dione 12 (900 mg, 5.079 mmol, crude). LCMS (210, 254 nm); tR=1.22 min, m/z=178.04 [M+H]⁺

To a solution of 6,7,8,9-tetrahydro-1H-cyclohepta[b]pyridine-2,5-dione (900 mg, 5.08 mmol) in acetonitrile (11 mL) was added dropwise POCl₃ (1.42 mL, 15.24 mmol) at room temperature. The reaction mixture was then stirred at 100° C. for 2 hour. The reaction mixture was concentrated, the flask was placed in an ice-bath, aqueous ammonium hydroxide was added dropwise slowly to quench the reaction mixture, EtOAc was added, organic layer was washed with water, dried over Na₂SO₄, and concentrated in vacuo. Purification by ISCO flash chromatography (4 g, 0-100% EtOAc in hexanes) gave a yellow solid of 2-chloro-6,7,8,9-tetrahydrocyclohepta[b]pyridin-5-one I (646 mg, 3.3018 mmol, 65% yield for 2 steps). Purity ≥95% by LCMS (210, 254 nm); tR=1.95 min, m/z=196.00 [M+H]⁺

To 2-chloro-6,7,8,9-tetrahydrocyclohepta[b]pyridin-5-one (308 mg, 1.57 mmol) in THE (8 mL) was added sodium hydride (346.33 mg, 8.66 mmol) at 0° C. and stirred for 15 min. After addition of MeI (0.44 mL, 7.08 mmol) at 0° C., the reaction mixture was stirred at room temperature for 3 h. The reaction was quenched with water slowly, diluted with EtOAc, extracted with EtOAc, dried over Na₂SO₄ and concentrated in vacuo. Purification by ISCO flash chromatography (12 g, 20% EtOAc in hexanes) gave a white solid of 2-chloro-6,6-dimethyl-8,9-dihydro-7H-cyclohepta[b]pyridin-5-one J (219 mg, 0.9790 mmol, 62% yield). Purity ≥95% by LCMS (210, 254 nm); tR=1.54 m/z=224.04 [M+H]⁺

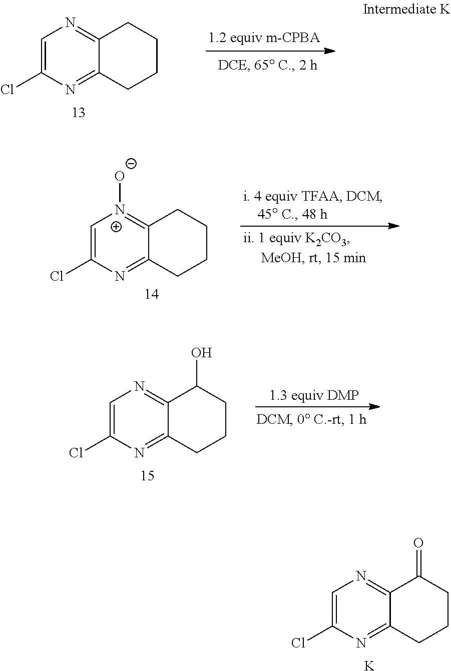

To 2-chloro-5,6,7,8-tetrahydroquinoxaline (814 mg, 4.83 mmol) in DCE (16 mL) was added m-chloroperbenzoic acid (1.33 g, 5.79 mmol) at room temperature and stirred at 65° C. for 2 h. Dilute the reaction mixture with DCM, wash with sat. aq. K₂CO₃ two times. The organic layer was dried over Na₂SO₄, filtered and concentrated in vacuo to get an off-white solid of 3-chloro-1-oxido-5,6,7,8-tetrahydroquinoxalin-1-ium 14 (855 mg, 4.6311 mmol, 95% yield). Purity ≥95% by LCMS (210, 254 nm); tR=1.57 m/z=185.04 [M+H]⁺

A mixture of 3-chloro-1-oxido-5,6,7,8-tetrahydroquinoxalin-1-ium (840 mg, 4.55 mmol) and trifluoroacetic anhydride (2.53 mL, 18.2 mmol) in DCM (16 mL) was stirred at 45° C. for 48 h. The reaction mixture was concentrated, the residue was re-dissolved in MeOH, potassium carbonate (628.84 mg, 4.55 mmol) was added and stirred for 15 min at room temperature. The solvent was evaporated, diluted with EtOAc and water, the aqueous layer was extracted with EtOAc, dried over Na₂SO₄ and concentrated in vacuo. Purification by ISCO flash chromatography (40 g, 60% EtOAc in hexanes) gave a colorless liquid of 2-chloro-5,6,7,8-tetrahydroquinoxalin-5-ol 15 (200 mg, 1.0833 mmol, 23% yield). Purity ≥95% by LCMS (210, 254 nm); tR=1.44 m/z=167.03 [M+H—H₂O]⁺

To 2-chloro-5,6,7,8-tetrahydroquinoxalin-5-ol (183 mg, 0.99 mmol) in DCM (10 mL) was added Dess-Martin periodinane (546.54 mg, 1.29 mmol) at room temperature and stirred for 1.5 h. The reaction mixture was diluted with DCM, washed with sat. aq. NaHCO₃, the organic layer was separated, dried over Na₂SO₄ and concentrated in vacuo. Purification by ISCO flash chromatography (24 g, 50% EtOAc in hexanes) gave a white solid of 2-chloro-7,8-dihydro-6H-quinoxalin-5-one K (145 mg, 0.7940 mmol, 80% yield). Purity ≥95% by LCMS (210, 254 nm); tR=1.44 m/z=183.03 [M+H]⁺

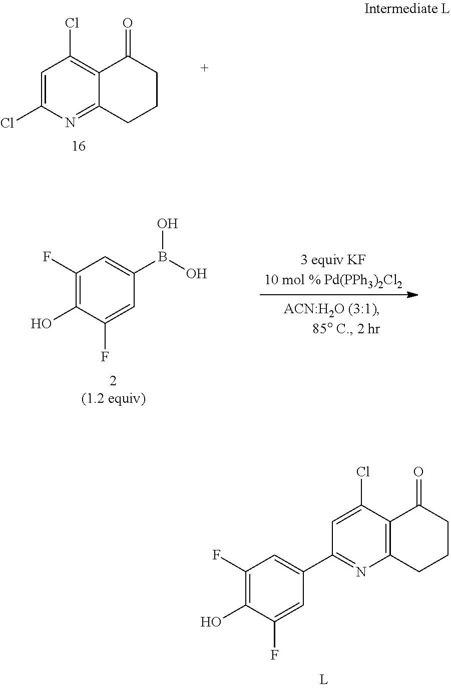

A mixture of 3,5-difluoro-4-hydroxyphenylboronic acid (482.95 mg, 2.78 mmol), 2,4-dichloro-7,8-dihydro-6H-quinolin-5-one (500 mg, 2.31 mmol), and Potassium fluoride (403.36 mg, 6.94 mmol) were dissolved in Acetonitrile (6 mL) and Water (2 mL). The reaction mixture was then degassed for 5 mins before adding bis(triphenylphosphine)palladium(II) dichloride (162.9 mg, 0.23 mmol). The reaction mixture was stirred at 85° C. for 2 h. After completion the mixture was diluted with EtOAc, poured into the separating funnel containing sat. aq. NaHCO₃, and the organic layer was separated. The combined organic layers were dried over Na₂SO₄ and concentrated in vacuo. Purification by ISCO flash chromatography (40 g, 0-30% EtOAc in hexanes) gave a yellow solid of 4-chloro-2-(3,5-difluoro-4-hydroxy-phenyl)-7,8-dihydro-6H-quinolin-5-one L (393 mg, 1.269 mmol, 54% yield). Purity ≥85% by LCMS (210, 254 nm); tR=1.24 min, m/z=310.04 [M+H]⁺

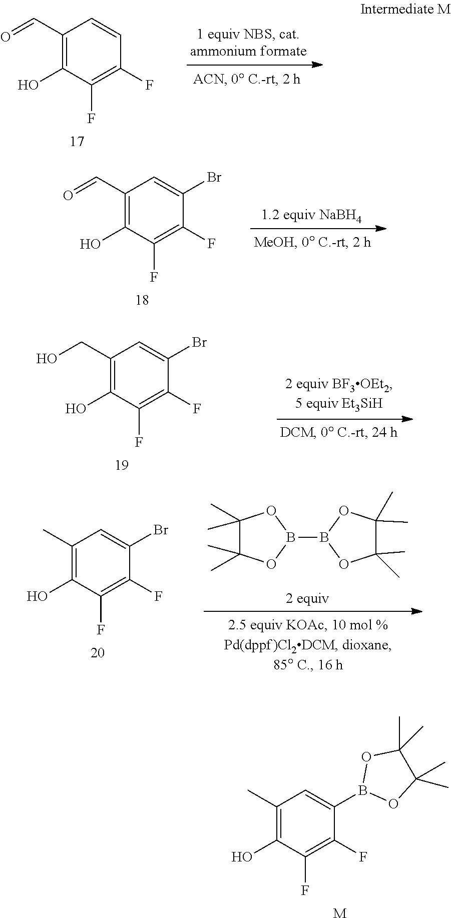

To a solution of 3,4-difluoro-2-hydroxy-benzaldehyde (1.14 g, 7.18 mmol) in acetonitrile (20 mL) was added N-bromosuccinimide (1.28 g, 7.18 mmol) and ammonium formate (45.27 mg, 0.72 mmol) at 0° C. The reaction was stirred to room temperature for 3 h. The solvent was evaporated, the crude mass was diluted with DCM and washed with water. The organic layer was separated, dried over Na₂SO₄ and concentrated in vacuo to get yellow solid of 5-bromo-3,4-difluoro-2-hydroxy-benzaldehyde 18 (1.7 g, 7.173 mmol, crude). In LCMS no ionization of product is observed. ¹H NMR (400 MHZ, MeOD) δ 10.09 (s, 1H), 7.80 (dd, J=7.3, 3.2 Hz, 1H). ¹⁹F NMR (376 MHz, MeOD) δ −121.98 (d, J=18.6 Hz, 1F), −158.66 (d, J=18.6 Hz, 1F).

To a solution of 5-bromo-3,4-difluoro-2-hydroxy-benzaldehyde (1.7 g, 7.17 mmol) in methanol (15 mL) was added NaBH₄ (1.82 g, 8.61 mmol) at 0° C. very slowly over 30 min. The reaction was warmed to room temperature and stirred for 2 h. Partially MeOH was removed and the reaction mixture was diluted with EtOAc. After acidifying the mixture with 1N HCl, the organic layer was separated, washed with brine, dried over Na₂SO₄, concentrated in vacuo. Purification by ISCO flash chromatography (80 g, 60% EtOAc in hexanes) gave a pink solid of 4-bromo-2,3-difluoro-6-(hydroxymethyl)phenol 19 (1.10 g, 4.4768 mmol, 60% yield for 2 steps). In LCMS no ionization of product is observed. ¹H NMR (400 MHZ, MeOD) δ 7.37-7.30 (m, 1H), 4.62 (s, 2H). ¹⁹F NMR (376 MHz, MeOD) δ −134.58 (d, J=20.1 Hz), −160.40 (d, J=20.1 Hz).

To a solution of 4-bromo-2,3-difluoro-6-(hydroxymethyl)phenol (1.10 g, 4.84 mmol) and triethylsilane (3.87 mL, 24.21 mmol) in DCM (20 mL) was added BF₃·OEt₂ (2.57 mL, 9.68 mmol) at 0° C. After stirring at 0° C. for 10 min, the reaction was warmed to RT and stirred for 20 h. The reaction mixture was poured into ice-water, extracted with EtOAc, the combined organic layers were washed with brine, dried over Na₂SO₄, concentrated in vacuo. Purification by ISCO flash chromatography (40 g, 15% EtOAc in hexanes) gave a white solid of 4-bromo-2,3-difluoro-6-methyl-phenol 20 (690 mg, 3.094 mmol, 63% yield). LCMS no ionization of product is observed. ¹H NMR (400 MHZ, MeOD) δ 7.09 (d, J=7.2 Hz, 1H), 2.17 (s, 3H). ¹⁹F NMR (376 MHz, MeOD) δ −136.42 (d, J=20.2 Hz, 1F), −160.51 (d, J=20.2 Hz, 1F).

4-Bromo-2,3-difluoro-6-methyl-phenol (250 mg, 1.12 mmol), bis(pinacolato)diboron (569.35 mg, 2.24 mmol), Pd(dppf)Cl₃·DCM (91.32 mg, 0.11 mmol) and KOAc (275.07 mg, 2.8 mmol) were combined in dioxane (8 mL) in a sealed tube and degassed under a stream of Ar for 10 mins then heated at 85° C. for 16 h. Reaction was diluted with EtOAc, washed with water, organic layer was dried over Na₂SO₄ and concentrated in vacuo. Purification by ISCO flash chromatography (24 g, 15% EtOAc in hexanes) gave a yellow solid of 2,3-difluoro-6-methyl-4-(4,4,5,5-tetramethyl-1,3,2-dioxaborolan-2-yl)phenol M (415 mg, 0.7683 mmol, 50% pure). ¹H NMR (400 MHz, MeOD) δ 7.14 (d, J=6.0 Hz, 1H), 2.16 (s, 3H), 1.33 (s, 12H). ¹⁹F NMR (376 MHz, MeOD) δ −133.76 (dd, J=20.3, 2.4 Hz, 1F), −166.00 (dd, J=20.3, 2.4 Hz, 1F).

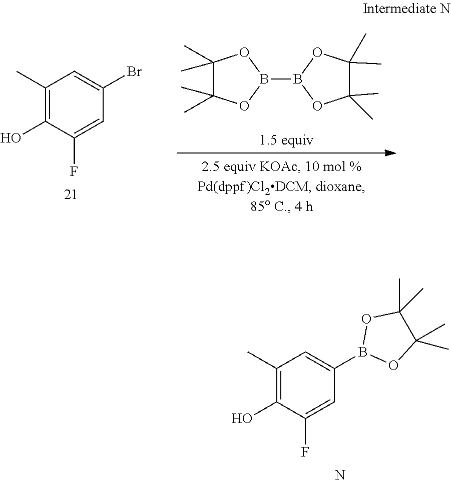

4-Bromo-2-fluoro-6-methyl-phenol (193 mg, 0.94 mmol), bis(pinacolato)diboron (358.58 mg, 1.41 mmol), Pd(dppf)Cl₂·DCM (76.69 mg, 0.09 mmol) and KOAc (230.99 mg, 2.35 mmol) were combined in dioxane (8 mL) in a sealed tube and degassed under a stream of Ar for 10 mins then heated at 85° C. for 4 h. Reaction was diluted with EtOAc, washed with water, organic layer was dried over Na₂SO₄ and concentrated in vacuo. Purification by ISCO flash chromatography (40 g, 5% EtOAc in hexanes) gave a white solid of 2-fluoro-6-methyl-4-(4,4,5,5-tetramethyl-1,3,2-dioxaborolan-2-yl)phenol N (142 mg, 0.5633 mmol, 59% yield). Purity ≥95% by LCMS (210); tR=1.65 min, m/z=253.14 [M+H]⁺

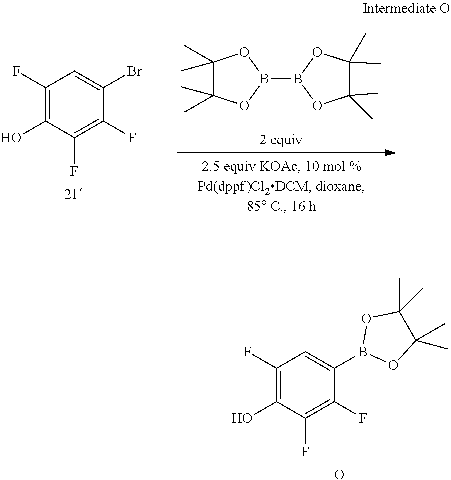

4-Bromo-2,3,6-trifluoro-phenol (366 mg, 1.61 mmol), bis(pinacolato)diboron (818.94 mg, 3.22 mmol), Pd(dppf)Cl₃·DCM (131.36 mg, 0.16 mmol) and KOAc (395.66 mg, 4.03 mmol) were combined in dioxane (8 mL) in a sealed tube and degassed under a stream of Ar for 10 mins then heated at 85° C. for 16 h. Reaction was diluted with EtOAc, washed with water, organic layer was dried over Na₂SO₄ and concentrated in vacuo. Purification by ISCO flash chromatography (40 g, 15% EtOAc in hexanes) gave a yellow solid of 2,3,6-trifluoro-4-(4,4,5,5-tetramethyl-1,3,2-dioxaborolan-2-yl)phenol 0) (227 mg, 0.5798 mmol, 70% purity). Purity ≥70% by LCMS (210, 254); tR=2.12 min, m/z=274.64 [M+H]⁺

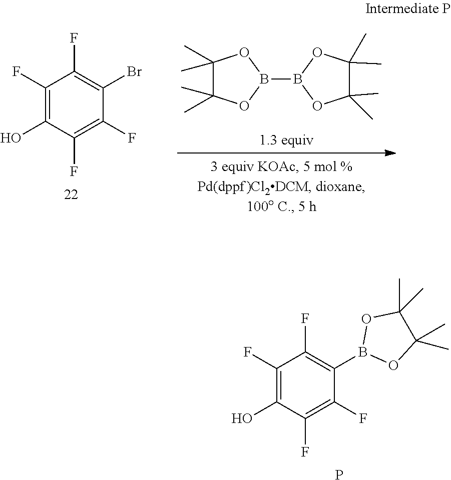

4-Bromo-2,3,5,6-tetrafluoro-phenol (808.4 mg, 3.3 mmol), bis(pinacolato)diboron (1.1 g, 4.29 mmol), Pd(dppf)Cl₂·DCM (134.41 mg, 0.17 mmol) and KOAc (971.69 mg, 9.9 mmol) were combined in dioxane (16 mL) in a sealed tube and degassed under a stream of Ar for 10 mins then heated at 100° C. for 5 h. Reaction was diluted with EtOAc, washed with water, organic layer was dried over Na₂SO₄ and concentrated in vacuo. Purification by ISCO flash chromatography (24 g, 10% EtOAc in hexanes) gave a white solid of 2,3,5,6-tetrafluoro-4-(4,4,5,5-tetramethyl-1,3,2-dioxaborolan-2-yl)phenol P (500 mg, 1.71 mmol, 51% yield). LCMS no ionization of product is observed. ¹H NMR (400 MHZ, CDCl3) δ 1.25 (s, 12H). ¹⁹F NMR (376 MHz, CDCl₃) δ −134.96-−135.09 (m, 2F), −160.54-−160.71 (m, 2F).

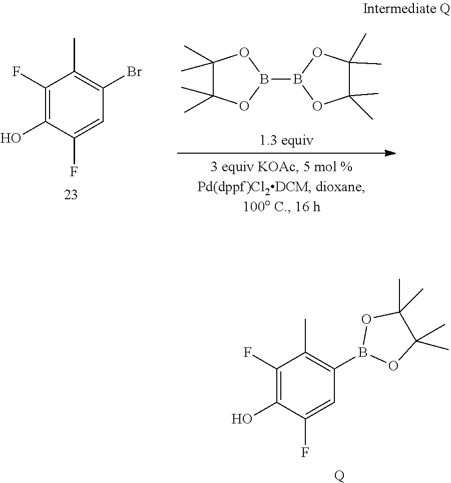

4-Bromo-2,6-difluoro-3-methylphenol (446.02 mg, 2.0 mmol), bis(pinacolato)diboron (660.24 mg, 2.6 mmol), Pd(dppf)Cl₂·DCM (81.46 mg, 0.1 mmol) and KOAc (588.9 mg, 6.0 mmol) were combined in dioxane (10 mL) in a sealed tube and degassed under a stream of Ar for 10 mins then heated at 100° C. for 16 h. Reaction was diluted with EtOAc, washed with water, organic layer was dried over Na2SO4 and concentrated in vacuo. Purification by ISCO flash chromatography (24 g, 10% EtOAc in hexanes) gave a white solid of 2,6-difluoro-3-methyl-4-(4,4,5,5-tetramethyl-1,3,2-dioxaborolan-2-yl)phenol Q (356 mg, 1.31 mmol, 65% yield). LCMS no ionization of product is observed. ¹H NMR (400 MHZ, CDCl3) δ 7.31 (dd, J=10.5, 2.1 Hz, 1H), 2.42 (s, 3H), 1.33 (s, 12H). ¹⁹F NMR (376 MHz, CDCl₃) δ −139.08 (d, J=9.7 Hz), —140.52 (d, J=9.7 Hz).

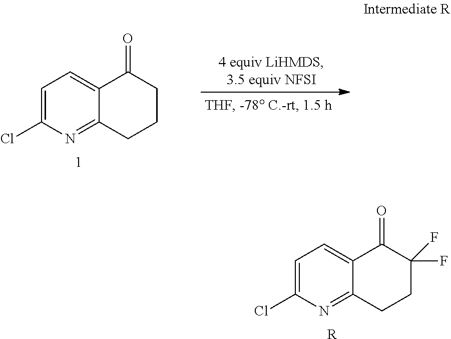

2-Chloro-7,8-dihydroquinolin-5(6H)-one (80 mg, 0.44 mmol) was taken in THE (4 mL) and cooled to −78° C. To this mixture was added LiHMDS (1.54 mL, 1.54 mmol) and then stirred at 0° C. for 40 min. The reaction mixture was again cooled to −78° C. followed by addition of N-fluorobenzenesulfonimide (555.6 mg, 1.76 mmol) dissolved in 1 mL THF. After stirring the reaction mixture for 20 min at −78° C., it was continued stirring for another 30 min at room temperature followed by quenching of the reaction mixture with sat. aq. NH₄Cl. The aqueous layer was extracted with Et₂O, the organic layer was dried over Na₂SO₄ and concentrated in vacuo. Purification by ISCO flash chromatography (12 g, 15% EtOAc in hexanes) gave a yellow solid of 2-chloro-6,6-difluoro-7,8-dihydroquinolin-5-one R (41 mg, 0.1884 mmol, 42% yield). Purity ≥95% by LCMS (210, 254 nm); tR=1.03 m/z=218.01 [M+H]⁺

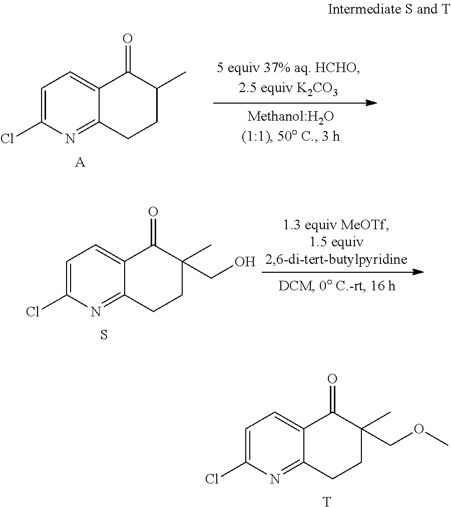

To 2-chloro-6-methyl-7,8-dihydro-6H-quinolin-5-one (170 mg, 0.87 mmol) in methanol (4.3 mL) was added K₂CO₃ (300.22 mg, 2.17 mmol) and water (4.3 mL) followed by addition of 37% formaldehyde (0.32 mL, 4.34 mmol). The reaction mixture was stirred vigorously at 50° C. for 3 h. MeOH was removed under reduced pressure, the reaction mixture was diluted with EtOAc, washed with water and brine, the organic layer was separated, dried over Na₂SO₄ and concentrated in vacuo. Purification by ISCO flash chromatography (12 g, 60% EtOAc in hexanes) gave a colorless liquid of 2-chloro-6-(hydroxymethyl)-6-methyl-7,8-dihydroquinolin-5-one S (185 mg, 0.8198 mmol, 94% yield). Purity ≥95% by LCMS (210, 254 nm); tR=0.60 m/z=226.06 [M+H]⁺

To Intermediate S (85 mg, 0.38 mmol) in DCM (1.2 mL) at 0° C. was added 2,6-di-tert-butyl pyridine (0.13 mL, 0.56 mmol) and methyl trifluoromethanesulfonate (0.06 mL, 0.49 mmol). The reaction mixture was stirred at room temperature for 16 h. The reaction mixture was quenched with water, the product was extracted with DCM, the organic layer was washed with sat. aq. NaHCO₃, dried over Na₂SO₄ and concentrated in vacuo. Purification by ISCO flash chromatography (12 g, 30% EtOAc in hexanes) gave a thick colorless liquid of 2-chloro-6-(methoxymethyl)-6-methyl-7,8-dihydroquinolin-5-one T (5 mg, 0.0209 mmol, 5% yield). Purity ≥95% by LCMS (210, 254 nm); tR=2.12 min, m/z=240.30 [M+H]⁺

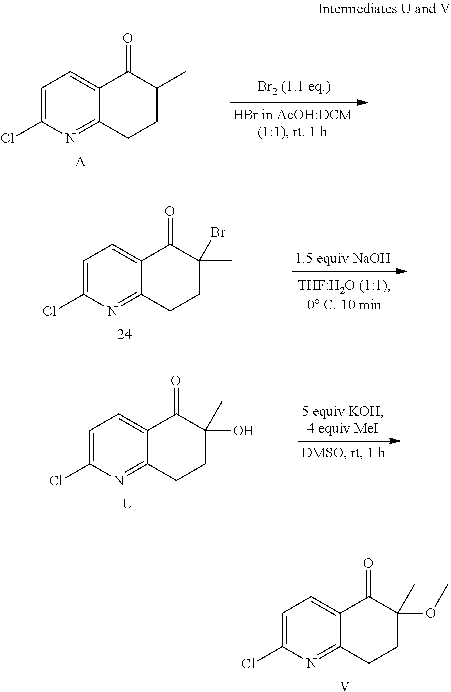

To 2-chloro-6-methyl-7,8-dihydro-6H-quinolin-5-one (272 mg, 1.39 mmol) in HBr in acetic acid (2.7 mL) and DCM (2.7 mL) was added bromine (0.08 mL, 1.53 mmol) at room temperature and stirred for 1 h at the same temperature. The reaction was quenched with ice-water, extracted with DCM, the combined organic layers were washed with sat. aq. NaHCO₃, dried over Na₂SO₄ and concentrated in vacuo. Purification by ISCO flash chromatography (24 g, 15% EtOAc in hexanes) gave a white solid of 6-bromo-2-chloro-6-methyl-7,8-dihydroquinolin-5-one 24 (360 mg, 1.3113 mmol, 94% yield). Purity ≥95% by LCMS (210, 254 nm); tR=1.61 min, m/z=275.73 [M+H]⁺

To a solution of 6-bromo-2-chloro-6-methyl-7,8-dihydroquinolin-5-one (110 mg, 0.4 mmol) in THF (4 mL) and water (4 mL) was added NaOH (24.04 mg, 0.6 mmol) at 0° C. and stirred for 10 min at the same temperature. The reaction was quenched with sat. aq. NH₄Cl, extracted with EtOAc, the organic layer was dried over Na₂SO₄ and concentrated in vacuo. Purification by ISCO flash chromatography (12 g, 45% EtOAc in hexanes) gave an off-white solid of 2-chloro-6-hydroxy-6-methyl-7,8-dihydroquinolin-5-one U (79 mg, 0.3733 mmol, 93% yield). Purity ≥95% by LCMS (210, 254 nm); tR=0.65 min, m/z=212.04 [M+H]⁺

To 2-chloro-6-hydroxy-6-methyl-7,8-dihydroquinolin-5-one (74 mg, 0.35 mmol) in DMSO (3.5 mL) was added potassium hydroxide (98.08 mg, 1.75 mmol) followed by methyl iodide (0.09 mL, 1.4 mmol) at room temperature. The reaction was stirred for 1 h at the same temperature. Water was added to the reaction mixture and extracted with EtOAc, the combined organic layers were washed with brine, dried over Na₂SO₄ and concentrated in vacuo, Purification by ISCO flash chromatography (12 g, 20% EtOAc in hexanes) gave a thick colorless liquid of 2-chloro-6-methoxy-6-methyl-7,8-dihydroquinolin-5-one V (44 mg, 0.1950 mmol, 55% yield), Purity ≥85% by LCMS (210, 254 nm); tR=1.34 min, m/z=226.24 [M+H]⁺

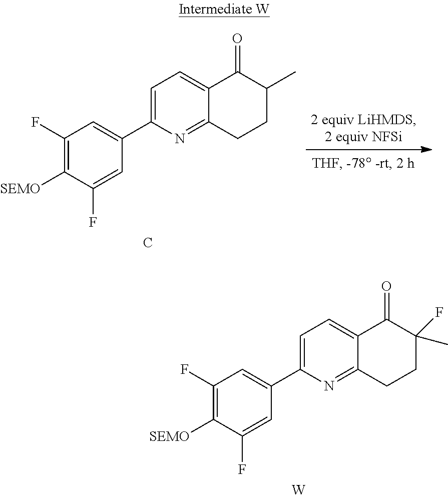

2-[3,5-difluoro-4-(2-trimethylsilylethoxymethoxy)phenyl]-6-methyl-7,8-dihydro-6H-quinolin-5-one (101 mg, 0.24 mmol) was taken in THF (2 mL) and cooled to −78° C. To this solution was added LiHMDS (0.48 mL, 0.48 mmol) and then stirred at 0° C. for 50 min. The reaction mixture was again cooled to −78° C. followed by addition of N-fluorobenzenesulfonimide (151.83 mg, 0.48 mmol) dissolved in 2 mL THF. After stirring the reaction for 10 min at −78° C., stirring was continued for another 1 h at room temperature. The reaction mixture was quenched with sat. aq. NH₄Cl, extracted with EtOAc, organic layer was separated, dried over Na₂SO₄ and concentrated in vacuo. Purification by ISCO flash chromatography (12 g, 15% EtOAc in hexanes) gave a yellow solid of 2-[3,5-difluoro-4-(2-trimethylsilylethoxymethoxy)phenyl]-6-fluoro-6-methyl-7,8-dihydroquinolin-5-one W (91 mg, 0.2080 mmol, 86% yield). Purity ≥95% by LCMS (210, 254 nm); tR=2.74 m/z=438.26 [M+H]⁺

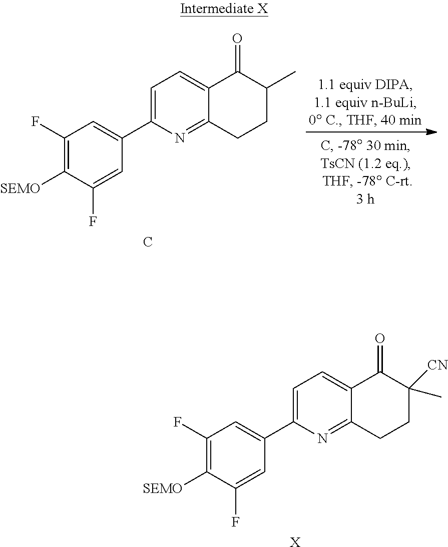

A mixture of diisopropylamine (0.03 mL, 0.19 mmol) and n-BuLi (0.08 mL, 0.19 mmol) in 1.3 mL THF was stirred at 0° C. for 40 min. To this solution was added 2-[3,5-difluoro-4-(2-trimethylsilylethoxymethoxy)phenyl]-6-methyl-7,8-dihydro-6H-quinolin-5-one (72 mg, 0.17 mmol) dissolved 1.2 mL THF at −78° C. and stirred for 30 min followed by addition of tosyl cyanide (37.32 mg, 0.21 mmol) in 1 mL of THF. The reaction mixture was stirred for 1 h at −78° C. followed by stirring at room temperature for 2 h. The reaction mixture was quenched with sat. aq. NH₄Cl, extracted with EtOAc, organic layer was separated, dried over Na₂SO₄ and concentrated in vacuo. Purification by ISCO flash chromatography (12 g, 10% EtOAc in hexanes) gave an off-white solid 2-[3,5-difluoro-4-(2-trimethylsilylethoxymethoxy)phenyl]-6-methyl-5-oxo-7,8-dihydroquinoline-6-carbonitrile X (27 mg, 0.0607 mmol, 35% yield). Purity ≥95% by LCMS (210, 254 nm); tR=2.64 m/z=445.17 [M+H]⁺

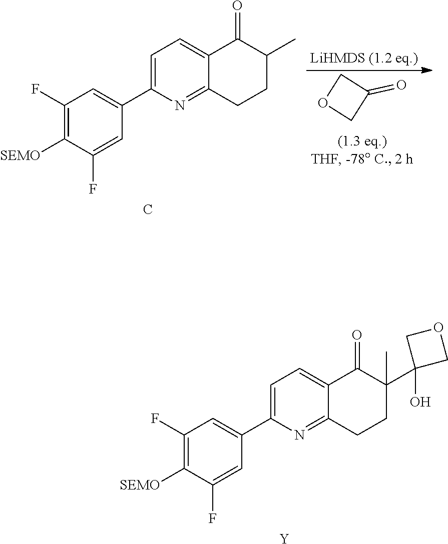

To a solution of 2-[3,5-difluoro-4-(2-trimethylsilylethoxymethoxy)phenyl]-6-methyl-7,8-dibydro-6H-quinolin-5-one (255 mg, 0.61 mmol) in THE (5 mL) was added LiHMDS (0.73 mL, 0.73 mmol) at −78° C. After stirring for 1 h, 3-oxetanone (0.05 mL, 0.79 mmol) was added and continued stirring at −78° C. for another 1 h. The reaction mixture was quenched with sat. aq. NH₄Cl, extracted with EtOAc, dried over Na₂SO₄ and concentrated in vacuo. Purification by ISCO flash chromatography (24 g, 60% EtOAc in hexanes) gave a white solid of 2-[3,5-difluoro-4-(2-trimethylsilylethoxymethoxy)phenyl]-6-(3-hydroxyoxetan-3-yl)-6-methyl-7,8-dihydroquinolin-5-one Y (230 mg, 0.4679 mmol, 76% yield). Purity ≥95% by LCMS (210, 254 nm); tR=2.30 m/z 492.20 [M+H]⁺

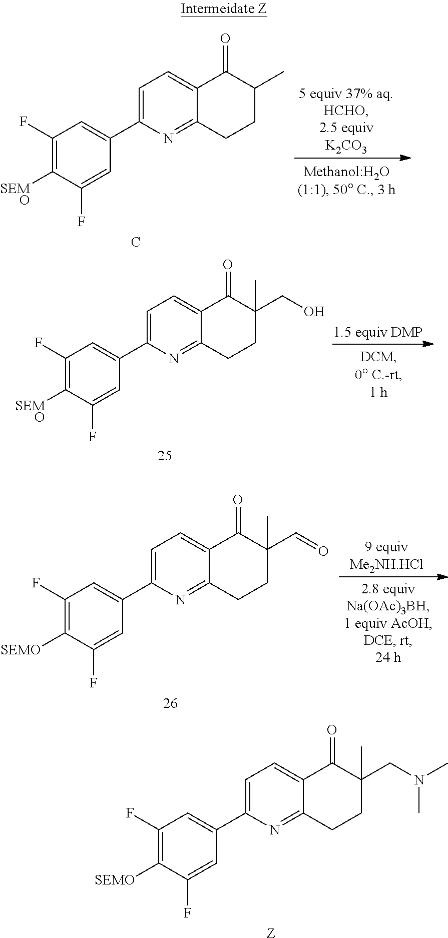

To 2-[3,5-difluoro-4-(2-trimethylsilylethoxymethoxy)phenyl]-6-methyl-7,8-dihydro-6H-quinolin-5-one (87 mg, 0.21 mmol) in methanol (2.5 mL) was added K₂CO₃ (71.65 mg, 0.52 mmol) and water (2.5 mL) followed by addition of 37% formaldehyde (0.08 mL, 1.04 mmol). The reaction mixture was stirred vigorously at 50° C. for 3 h. MeOH was removed under reduced pressure, the reaction mixture was diluted with EtOAc, washed with water and brine, the organic layer was separated, dried over Na₂SO₄ and concentrated in vacuo. Purification by ISCO flash chromatography (12 g, 30% EtOAc in hexanes) gave a white solid of 2-[3,5-difluoro-4-(2-trimethylsilylethoxymethoxy)phenyl]-6-(hydroxymethyl)-6-methyl-7,8-dihydroquinolin-5-one 25 (40 mg, 0.0890 mmol, 42% yield). Purity ≥95% by LCMS (210, 254 nm); tR=2.43 m/z=450.18 [M+H]⁺

To 2-[3,5-difluoro-4-(2-trimethylsilylethoxymethoxy)phenyl]-6-(hydroxymethyl)-6-methyl-7,8-dihydroquinolin-5-one (40 mg, 0.09 mmol) in DCM (2 mL) was added Dess-Martin periodinane (56.61 mg, 0.13 mmol) at 0° C. and stirred at room temperature for 1 h. The reaction was quenched with sat. aq. NaHCO₃, extracted with DCM, the organic layer was separated, dried over Na₂SO₄ and concentrated in vacuo to get aldehyde 26. To this aldehyde (44 mg, 0.1 mmol) in DCE (1.5 mL) was added dimethylamine hydrochloride (72.15 mg, 0.88 mmol) and acetic acid (0.01 mL, 0.1 mmol) at room temperature. After stirring for 5 min, sodium triacetoxyborohydride (58.34 mg, 0.28 mmol) was added followed by stirring for 24 h. Sat. aq. NaHCO₃ was added to the reaction mixture, extracted with DCM, the organic layer was dried over Na₂SO₄ and concentrated in vacuo to get a sticky liquid of 2-[3,5-difluoro-4-(2-trimethylsilylethoxymethoxy)phenyl]-6-[(dimethylamino)methyl]-6-methyl-7,8-dihydroquinolin-5-one Z (40 mg, crude; used in the next step directly). LCMS (210, 254 nm); tR=1.67, m/z=477.24 [M+H]⁺

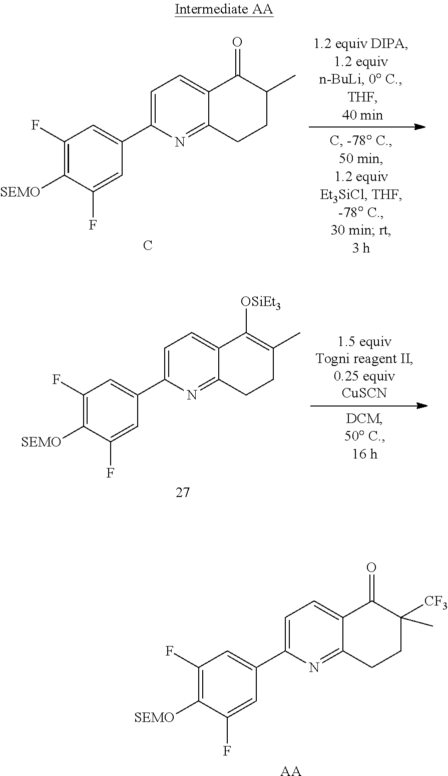

A mixture of diisopropylamine (0.02 mL, 0.16 mmol) and n-BuLi (0.07 mL, 0.16 mmol) in 1 mL THF was stirred at 0° C. for 40 min. To this solution was added 2-[3,5-difluoro-4-(2-trimethylsilylethoxymethoxy)phenyl]-6-methyl-7,8-dihydro-6H-quinolin-5-one (57 mg, 0.14 mmol) dissolved 1 mL THF at −78° C. and stirred for 50 min followed by addition of chloro(triethyl)silane (0.03 mL, 0.16 mmol) in 0.2 mL of THE at the same temperature. The reaction mixture was stirred for 30 min at −78° C. followed by stirring at room temperature for 3 h. The reaction mixture was quenched with sat. aq. NaHCO₃ at 0° C., extracted with EtOAc, the organic layer was separated, dried over Na₂SO₄ and concentrated in vacuo. Purification by ISCO flash chromatography (4 g, 0-100 EtOAc in hexanes with 0.1% Et₃N) gave a thick liquid of 2-[[2,6-difluoro-4-(6-methyl-5-triethylsilyloxy-7,8-dihydroquinolin-2-yl)phenoxy]methoxy]ethyl-trimethyl-silane 27 (50 mg, 0.0937 mmol, 68% yield). ¹H NMR (400 MHz, MeOD) δ 7.72-7.65 (m, 4H), 5.25 (s, 2H), 3.91 (t, J=8.4 Hz, 2H), 2.97 (t, J=8.0 Hz, 2H), 2.41 (t, J=8.0 Hz, 2H), 1.88 (s, 3H), 1.01 (t, J=8.0 Hz, 9H), 0.93 (t, J=8.4 Hz, 2H), 0.75 (q, J=8.0 Hz, 6H), 0.02 (s, 9H). ¹⁹F NMR (376 MHz, MeOD) δ −129.46.

To CuSCN (2.79 mg, 0.02 mmol) and 1-trifluoromethyl-1,2-benziodoxol-3-(1H)-one (78.32 mg, 0.25 mmol) under nitrogen was added 2-[[2,6-difluoro-4-(6-methyl-5-triethylsilyloxy-7,8-dihydroquinolin-2-yl)phenoxy]methoxy]ethyl-trimethyl-silane (49 mg, 0.09 mmol) dissolved in DMF (1.2 mL). The reaction mixture was stirred at 50° C. for 16 h. The reaction mixture was quenched with sat. aq. NH₄Cl, extracted with EtOAc, organic layer was separated, dried over Na₂SO₄ and concentrated in vacuo. Purification by ISCO flash chromatography (12 g, 10% EtOAc in hexanes) gave an off-white solid of 2-[3,5-difluoro-4-(2-trimethylsilylethoxymethoxy)phenyl]-6-methyl-6-(trifluoromethyl)-7,8-dihydroquinolin-5-one AA (12 mg, 0.0246 mmol, 26% yield). Purity ≥95% by LCMS (210, 254 nm); tR=3.32 m/z=488.26 [M+H]⁺

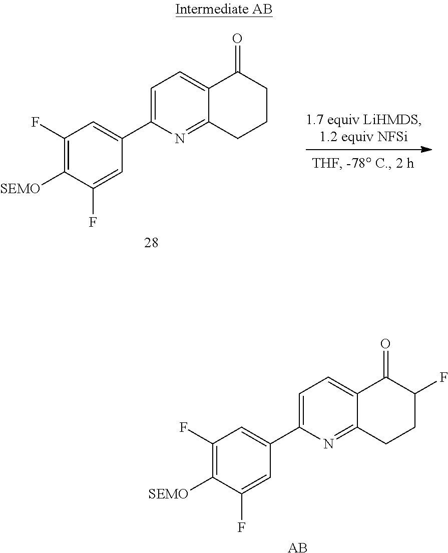

Compound 28 (prepared similar to Intermediate C) (120 mg, 0.3 mmol) was taken in THF (2 mL) and cooled to −78° C. To this solution was added LiHMDS (0.5 mL, 0.5 mmol) and then stirred for 1 h at the same temperature followed by addition of N-fluorobenzenesulfonimide (111.98 mg, 0.36 mmol) dissolved in 2 mL THF. After stirring the reaction for 1 h at −78° C., it was quenched with sat. aq. NH₄Cl, extracted with EtOAc, organic layer was separated, dried over Na₂SO₄ and concentrated in vacuo. Purification by ISCO flash chromatography (12 g, 15% EtOAc in hexanes) gave a yellow solid of 2-[3,5-difluoro-4-(2-trimethylsilylethoxymethoxy)phenyl]-6-fluoro-7,8-dihydro-6H-quinolin-5-one AB (109 mg, 0.2574 mmol, 86% yield). Purity ≥95% by LCMS (210, 254 nm); tR=2.56 m/z=424.13 [M+H]⁺

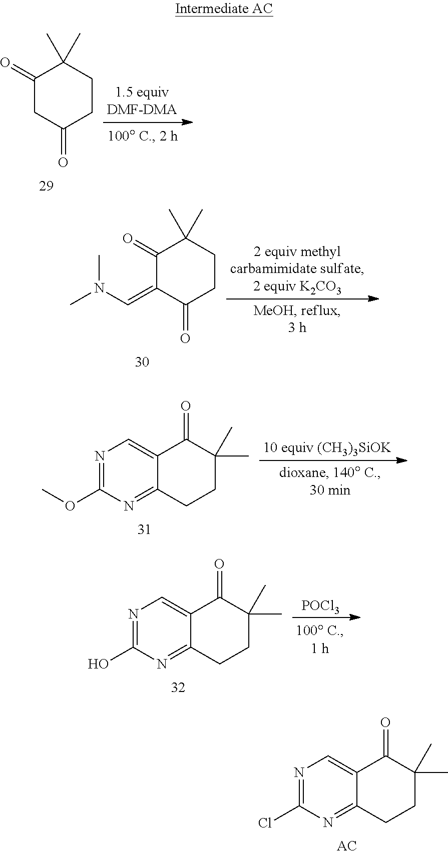

A mixture of 3-hydroxy-6,6-dimethyl-cyclohex-2-en-1-one (5 g, 35.67 mmol) and N,N-dimethylformamide dimethyl acetal (7.11 mL, 53.5 mmol) was stirred at 100° C. for 2 h. After evaporation, the reaction mixture was purified by ISCO flash chromatography (80 g, 0-10% MeOH in DCM) to get a brown solid of (2E)-2-(dimethylaminomethylene)-4,4-dimethyl-cyclohexane-1,3-dione 30 (6.12 g, 31.343 mmol, 87% yield). Purity ≥95% by LCMS (210, 254 nm); tR=1.34 m/z=196.13 [M+H]⁺

To (2E)-2-(dimethylaminomethylene)-4,4-dimethyl-cyclohexane-1,3-dione (2.7 g, 13.83 mmol) in methanol (12 mL) was added methyl carbamimidate sulfate (4.76 g, 27.66 mmol) and potassium carbonate (3.82 g, 27.66 mmol), and refluxed for 3 h. Water and EtOAc were added, the organic layer was separated, the aqueous layer was extracted with EtOAc two times, the combined organic layers were dried over Na₂SO₄ and concentrated in vacuo. Purification by ISCO flash chromatography (40 g, 0-10% MeOH in DCM) gave a yellow solid of 2-methoxy-6,6-dimethyl-7,8-dihydroquinazolin-5(6H)-one 31 (222 mg, 1.08 mmol, 8% yield). Purity ≥95% by LCMS (210, 254 nm); tR=1.58 m/z=207.10 [M+H]⁺

To 2-methoxy-6,6-dimethyl-7,8-dihydroquinazolin-5-one (170 mg, 0.82 mmol) in dioxane (4.5 mL) was added potassium trimethylsilanolate (1.06 g, 8.24 mmol) at room temperature and stirred at 140° C. for 30 min. The reaction mixture concentrated, diluted with sat. aq. NH₄Cl and 10% MeOH/DCM, the aqueous layer was extracted with 10% MeOH/DCM multiple times. The combined organic layers were dried over Na₂SO₄ and concentrated in vacuo. Purification by ISCO flash chromatography (12 g, 7% of MeOH in DCM) gave an off-white solid of 2-hydroxy-6,6-dimethyl-7,8-dihydroquinazolin-5-one 32 (93 mg, 0.4838 mmol, 58% yield). Purity ≥95% by LCMS (210, 254 nm); tR=1.23, m/z=193.09 [M+H]⁺

A mixture of 6,6-dimethyl-7,8-dihydro-1H-quinazoline-2,5-dione (93 mg, 0.48 mmol) and POCl₃ (3 mL) in a vial was stirred at 100° C. for 1 hour. The reaction mixture was concentrated, ice water was added, the aqueous layer was extracted with DCM, the organic layer was separated, dried over Na₂SO₄ and concentrated in vacuo. Purification by ISCO flash chromatography (12 g, 20% EtOAc in hexanes) gave a white solid of 2-chloro-6,6-dimethyl-7,8-dihydroquinazolin-5-one AC (45 mg, 0.2136 mmol, 44% yield). Purity ≥95% by LCMS (210, 254 nm); tR=0.91 min, m/z=211.06 [M+H]⁺

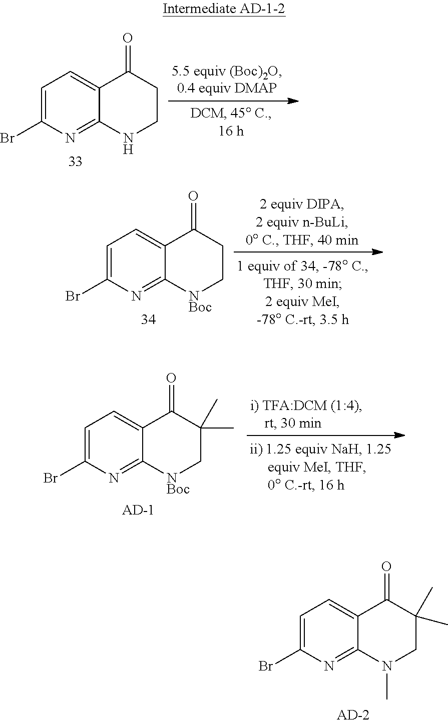

To 7-bromo-2,3-dihydro-1,8-naphthyridin-4 (1H)-one (163 mg, 0.72 mmol) in THF (2 mL) was added Boc anhydride (0.38 mL, 3.95 mmol) and DMAP (58.47 mg, 0.29 mmol) at room temperature and stirred at 45° C. for 16 h. The reaction was diluted with EtOAc and water, extracted with EtOAc, dried over Na₂SO₄ and concentrated in vacuo. Purification by ISCO flash chromatography (12 g, 20% EtOAc in hexanes) gave a white solid of tert-butyl 7-bromo-4-oxo-2,3-dihydro-1,8-naphthyridine-1-carboxylate 34 (62 mg, 0.1895 mmol, 26% yield). Purity ≥95% by LCMS (210, 254 nm); tR=1.62 m/z=270.97 [M+H-Boc]⁺

A mixture of diisopropylamine (0.05 mL, 0.35 mmol) and n-BuLi (0.14 mL, 0.35 mmol) in 1.5 mL THF at 0° C. was stirred for 40 min. To this solution was added tert-butyl 7-bromo-4-oxo-2,3-dihydro-1,8-naphthyridine-1-carboxylate (58 mg, 0.18 mmol) dissolved 1 mL THF at −78° C. and stirred for 30 min followed by addition of MeI (0.02 mL, 0.35 mmol). The reaction mixture was stirred for 15 min at −78° C. followed by stirring for 3.5 h at room temperature. The reaction mixture was quenched with sat. aq. NH₄Cl, extracted with EtOAc, dried over Na₂SO₄ and concentrated in vacuo. Purification by ISCO flash chromatography (4 g, 10% EtOAc in hexanes) gave a white solid of tert-butyl 7-bromo-3,3-dimethyl-4-oxo-2H-1,8-naphthyridine-1-carboxylate AD-1 (15 mg, 0.0422 mmol, 23% yield). Purity ≥95% by LCMS (210, 254 nm); tR=2.12 m/z=299.00 [M+H-Boc]⁺

Tert-butyl 7-bromo-3,3-dimethyl-4-oxo-2H-1,8-naphthyridine-1-carboxylate (50 mg, 0.140 mmol) was taken in 1:4 TFA and DCM (1 ml) and stirred for 30 min at room temperature. The solvent was evaporated to get solids which was subjected to hexane wash. The solids were re-dissolved in THF (1.5 ml) and cooled to 0° C. To this mixture was added sodium hydride (5 mg, 0.211 mmol) and stirred for 15 min at the same temperature followed by addition of methyl iodide (13 μL, 0.211 mmol). The reaction was stirred at room temperature for 16 h. Ice-water was added, the aqueous layer was extracted with EtOAc, the organic layer was separated, dried over Na₂SO₄ and concentrated in vacuo. Purification by ISCO flash chromatography (12 g, 15% EtOAc in hexanes) gave a white solid of 7-bromo-1,3,3-trimethyl-2H-1,8-naphthyridin-4-one AD-2 (21 mg, 0.078 mmol, 55% yield for 2 steps). Purity ≥95% by LCMS (210, 254 nm); tR=1.73 m/z=269.03 [M+H]⁺

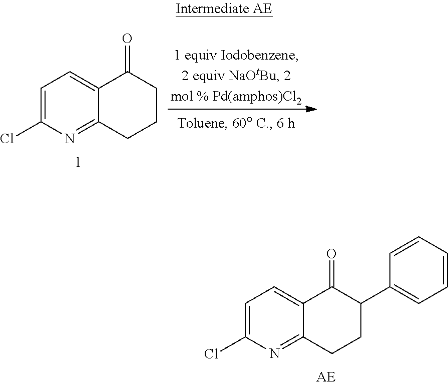 [SMILES: O=C1CCCc2nc(Cl)ccc21 ; O=C1c2ccc(Cl)nc2CCC1c1ccccc1]

To a stirred solution of 2-chloro-7,8-dihydroquinolin-5(6H)-one (500 mg, 2.75 mmol) and iodobenzene (561 mg, 2.75 mmol) in toluene (20 mL) was added sodium tert butoxide (528 mg, 5.50 mmol) and degassed the reaction mixture with argon for 15 min. To the resulting reaction mixture was added Pd(Amphos)Cl₂ (39 mg, 0.05 mmol) at room temperature and stirred for 5 min. The reaction mixture was heated at 60° C. for 6 h. The reaction mixture was quenched with water and extracted with EtOAc, the organic layer was washed with brine, dried over Na₂SO₄ and concentrated in vacuo. Purification by ISCO flash chromatography (24 g, 30% EtOAc in hexanes) gave an off-white solid of 2-chloro-6-phenyl-7,8-dihydroquinolin-5(6H)-one AE (170 mg, 0.659 mmol, 23% yield). Purity ≥95% by LCMS (210, 254 nm); tR=1.91 m/z=257.90 [M+H]⁺

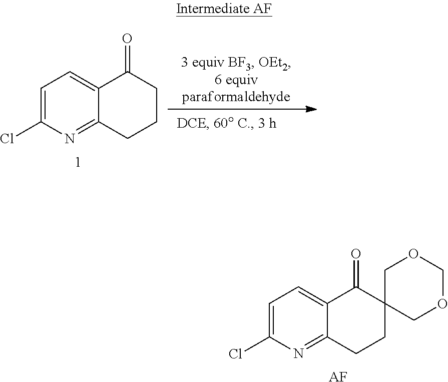

To a solution of 2-chloro-7,8-dihydroquinolin-5(6H)-one (200 mg, 1.10 mmol) in DCE (5 mL) was added paraformaldehyde (0.225 mL, 6.61 mmol) and BF₃·OEt₂ (0.407 ml, 3.30 mmol) at 0° C. and the reaction was stirred at 60° C. for 3 h. The reaction mixture was directly concentrated in vacuo. Purification by ISCO flash chromatography (12 g, 40% EtOAc in hexanes) gave a white solid of 2-chloro-7,8-dihydro-5H-spiro[quinoline-6,5′-[1,3]dioxan]-5-one AF (260 mg, 1.02 mmol, 93% yield). Purity ≥95% by LCMS (210, 254 nm); tR=0.74 m/z=254.05 [M+H]⁺

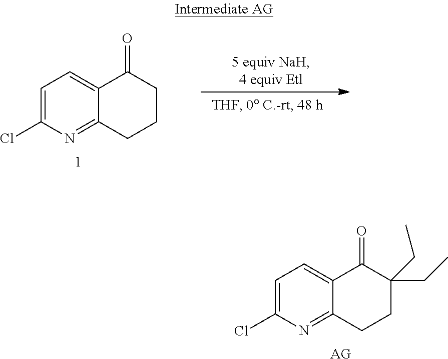

To 2-Chloro-7,8-dihydroquinolin-5(6H)-one (160 mg, 0.88 mmol) in THF (3 mL) was added sodium hydride (176.19 mg, 4.4 mmol) at 0° C. and stirred for 15 min. After addition of Ethyl iodide (0.22 mL, 3.52 mmol) at 0° C., the reaction mixture was stirred at room temperature for 48 h. The reaction was quenched with water, extracted with Et₂O, the organic layer was dried over Na₂SO₄ and concentrated in vacuo. Purification by ISCO flash chromatography (4 g, 5% EtOAc in hexanes) gave a yellow oil of 2-chloro-6,6-diethyl-7,8-dihydroquinolin-5-one AG (40 mg, 0.1683 mmol, 70% purity). LCMS (210, 254 nm); tR=1.65 m/z=238.09 [M+H]⁺

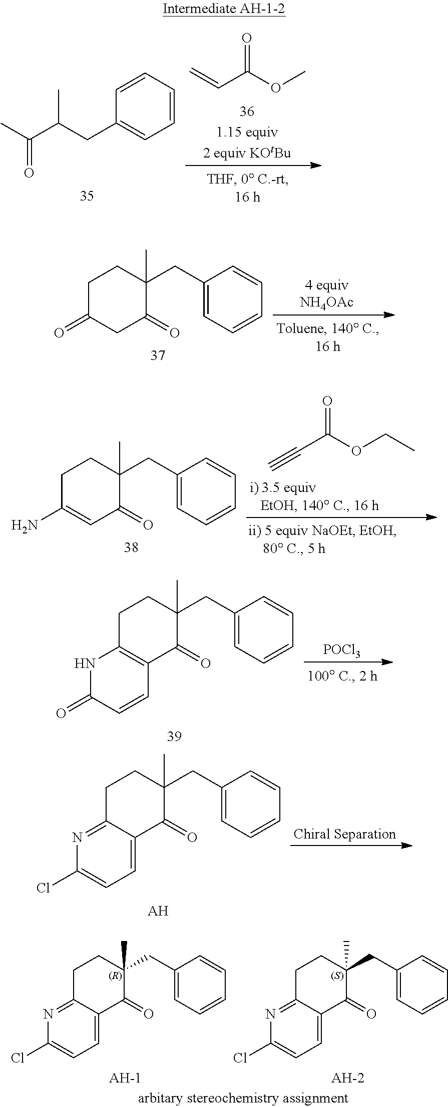

To a stirred solution of 3-methyl-4-phenylbutan-2-one (3.0 g, 18.49 mmol) in THF (75 mL) was added KO^{t}Bu (4.14 g, 36.96 mmol) at 0° C. and stirred for 15 min. To the resulting reaction mixture was added methyl acrylate (1.92 g, 22.32 mmol) at 0° C. and stirred at room temperature for 16 h. The reaction mixture was quenched with sat. aq. NH₄Cl and extracted with EtOAc, the organic layer was washed with brine, dried over Na₂SO₄ and concentrated in vacuo. Purification by ISCO flash chromatography (80 g, 50% EtOAc in hexanes) gave a yellow oil of 4-benzyl-4-methylcyclohexane-1,3-dione 37 (1.50 g, 6.94 mmol, 38% yield). MS (ESI) m/z [M+H]⁺: (Calculated. for: C₁₄H₁₆O₂: 216.28); Observed.: 216.98 [M+H]⁺

To a stirred solution of 4-benzyl-4-methylcyclohexane-1,3-dione (1.5 g, 6.944 mmol) in toluene (25 mL) was added ammonium acetate (2.13 g, 27.77 mmol) at room temperature and stirred for 5 min. The resulting reaction mixture was heated at 140° C. for 16 h. The reaction mixture was directly concentrated in vacuo. Purification by ISCO flash chromatography (40 g, 70% EtOAc in hexanes) gave a yellow solid of 3-amino-6-benzyl-6-methylcyclohex-2-en-1-one 38 (1.20 g, 5.57 mmol, 81% yield). MS (ESI) m/z [M+H]⁺: (Calculated. for: C₁₄H₁₇NO: 215.13); Observed.: 216.21 [M+H]⁺

To a stirred solution of 3-amino-6-benzyl-6-methylcyclohex-2-en-1-one (0.800 g, 3.720 mmol) in ethanol (4 mL) was added ethyl propiolate (6.0 mL, 13.07 mmol) at room temperature. The reaction mixture was heated at 140° C. and stirred for 16 h. After completion of the time, the reaction mixture was cooled at room temperature. To resulting reaction mixture was added ethanol (4 mL) followed by sodium ethoxide (6 mL, 18.60 mmol) at room temperature. The reaction mixture was heated at 80° C. for 5 h. The reaction mixture was directly concentrated in vacuo. Purification by ISCO flash chromatography (24 g, 80% EtOAc in hexanes) gave a yellow solid of 6-benzyl-6-methyl-7,8-dihydroquinoline-2,5 (1H,6H)-dione 39 (0.250 g, 0.935 mmol, 25% yield). MS (ESI) m/z [M+H]⁺: (Calculated. for: C₁₇H₁₇NO₂: 267.33); Observed.: 268.32 [M+H]⁺

To 6-benzyl-6-methyl-7,8-dihydroquinoline-2,5 (1H,6H)-dione (250 mg, 0.936 mmol) was added POCl₃ (2.5 mL) at room temperature. The reaction mixture was heated at 100° C. for 2 h. The reaction mixture was concentrated in vacuo, the obtained residue was dissolved in water and quenched with sat. aq. NaHCO₃. The aqueous layer was extracted with EtOAc, the organic layer was dried over Na₂SO₄ and concentrated in vacuo. Purification by ISCO flash chromatography (12 g, 70% EtOAc in hexanes) gave an off-white solid of 6-benzyl-2-chloro-6-methyl-7,8-dihydroquinolin-5(6H)-one AH (170 mg, 0.595 mmol, 64% yield) as a racemic mixture. Achiral HPLC: Peak-1: HPLC=49.63%, Rt=6.856 min; Peak-2: HPLC=50.36%, Rt=7.671 min

The resulting racemic mixture of AH (90 mg) was subjected to Chiral Prep HPLC to afford the colorless oils of AH-1 (Peak-1, 24.12 mg): MS (ESI) m/z [M+H]⁺: (Calculated. for: C₁₇H₁₆ClNO: 285.1); Observed.: 286.1 [M+H]⁺, HPLC: 100%, Rt=6.857 min, and AH-2 (Peak-2, 21.39 mg): MS (ESI) m/z [M+H]⁺: (Calculated. for: C₁₇H₁₆ClNO: 285.1); Observed.: 286.1 [M+H]⁺, HPLC: 99.55%, Rt=7.672 min.

### Chiral Prep HPLC Method:

Column: Chiral Pak-IG (30 mm×250 mm, 5μ),
Mobile Phase-A: 0.1% DEA in n-Hexane,
Mobile Phase-B: DCM: MeOH (1:1),
Mobile Phase-A: B-98:02,
Flow: 36 mL/min,
Loading: 16 mg/Inj/20 min

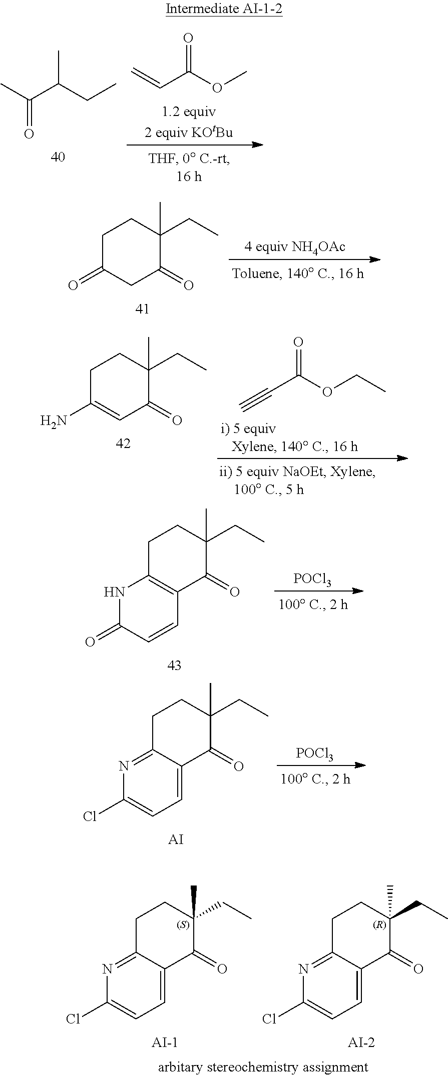

To a stirred solution of 3-methylpentan-2-one (10.0 g, 100 mmol) in THF (250 mL) was added KO^{t}Bu (22.40 g, 200 mmol) at 0° C. and stirred for 15 min. To the resulting reaction mixture was added methyl acrylate (10.32 g, 120 mmol) at 0° C. and stirred at room temperature for 16 h. The reaction mixture was quenched with sat. aq. NH₄Cl followed by 1M HCl (10 mL), extracted with EtOAc, the organic layer was washed with brine, dried over Na₂SO₄ and concentrated in vacuo. Purification by ISCO flash chromatography (120 g, 50% EtOAc in hexanes) gave a yellow oil of 4-ethyl-4-methylcyclohexane-1,3-dione 41 (5.80 g, 37.61 mmol, 37.66% yield). MS (ESI) m/z [M+H]⁺: (Calculated, for: C₉H₁₄O₂: 154.21); Observed.: 153.04 [M−1].

To a stirred solution of 4-ethyl-4-methylcyclohexane-1,3-dione (5.80 g, 37.66 mmol) in toluene (80 mL) was added ammonium acetate (11.5 g, 150.6 mmol) at room temperature and stirred for 5 min. The resulting reaction mixture was heated at 140° C. for 16 h. The reaction mixture was directly concentrated in vacuo. Purification by ISCO flash chromatography (80 g, 70% EtOAc in hexanes) gave a yellow solid of 3-amino-6-ethyl-6-methylcyclohex-2-en-1-one 42 (4.30 g, 28.06 mmol, 75% yield). MS (ESI) m/z [M+H]⁺: (Calculated. for: C₉H₁₅NO: 153.23); Observed.: 154.21 [M+H]⁺

To a stirred solution of 3-amino-6-ethyl-6-methylcyclohex-2-en-1-one (2.0 g, 13.07 mmol) in xylene (10 mL) was added ethyl propiolate (6.60 mL, 65.35 mmol) at room temperature. The reaction mixture was heated at 140° C. and stirred for 16 h. After completion of the time, the reaction mixture was cooled at room temperature. To resulting reaction mixture was added xylene (10 mL) followed by sodium ethoxide (21.0 mL, 65.35 mmol) at room temperature. The reaction mixture was heated at 100° C. for 5 h. The reaction mixture was directly concentrated in vacuo. Purification by ISCO flash chromatography (40 g, 70% EtOAc in hexanes) gave a yellow solid of 6-ethyl-6-methyl-7,8-dihydroquinoline-2,5(3H,6H)-dione 43 (1.10 g, 5.36 mmol, 41% yield). MS (ESI) m/z [M+H]⁺: (Calculated. for: C₁₂H₁₅NO₂: 205.26); Observed.: 206.32 [M+H]⁺

To 6-ethyl-6-methyl-7,8-dihydroquinoline-2,5(3H,6H)-dione (500 mg, 2.439 mmol) was added POCl₃ (5 mL) at room temperature. The reaction mixture was heated at 100° C. for 2 h. The reaction mixture was concentrated in vacuo, the obtained residue was dissolved in water and quenched with sat. aq. NaHCO₃. The aqueous layer was extracted with EtOAc, the organic layer was dried over Na₂SO₄ and concentrated in vacuo. Purification by ISCO flash chromatography (12 g, 50% EtOAc in hexanes) gave a thick yellow oil of 2-chloro-6-ethyl-6-methyl-7,8-dihydroquinolin-5(6H)-one AI (350 mg, 1.56 mmol, 64% yield).

The resulting racemic mixture of 2-chloro-6-ethyl-6-methyl-7,8-dihydroquinolin-5(6H)-one (350 mg) was subjected to Chiral Prep HPLC to afford the colorless oils of AI-1 (Peak-1, 120.83 mg): MS (ESI) m/z [M+H]⁺: (Calculated. for: C₁₂H₁₄ClNO: 223.70); Observed.: 224.1 [M+H]⁺, HPLC: 99.74%, Rt=8.778 min, and AI-2 (Peak-2, 169.85 mg): MS (ESI) m/z [M+H]⁺: (Calculated. for: C₁₂H₁₄ClNO: 223.70); Observed.: 224.1 [M+H]⁺, HPLC: 98.79%, Rt=8.515 min.

### Chiral Prep HPLC Method:

Column: Chiral Pak-IG (30 mm×250 mm, 5μ),
Mobile Phase-A: 0.1% DEA in n-Hexane,
Mobile Phase-B: DCM: MeOH (80:20),
Mobile Phase-A: B-98:02,
Flow: 36 mL/min,
Loading: 12 mg/Inj/20 min

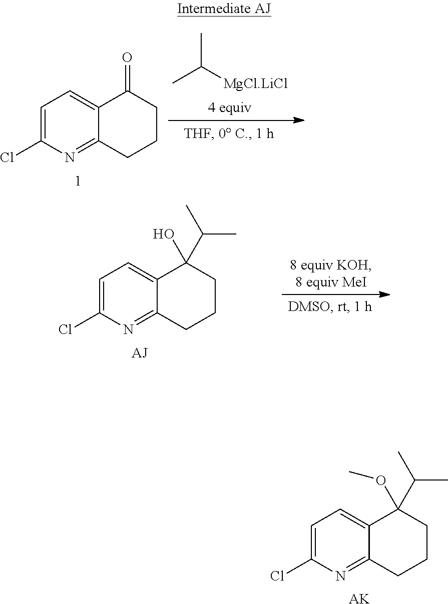

To 2-Chloro-7,8-dihydroquinolin-5(6H)-one (200 mg, 1.1 mmol) in THF (3.5 mL) at 0° C. was added chloro(isopropyl)magnesium;chlorolithium (3.39 mL, 4.4 mmol) and stirred for 1 h at the same temperature. The reaction mixture was quenched with sat. aq. NH₄Cl, extracted with EtOAc, the organic layer was dried over Na₂SO₄ and concentrated in vacuo. Purification by ISCO flash chromatography (12 g, 50% EtOAc in hexanes) gave a white solid of 2-chloro-5-isopropyl-7,8-dihydro-6H-quinolin-5-ol AJ (91 mg, 0.4032 mmol, 36% yield). Purity ≥95% by LCMS (210, 254 nm); tR=1.93 m/z=226.09 [M+H]⁺

To 2-chloro-5-isopropyl-7,8-dihydro-6H-quinolin-5-ol (91 mg, 0.4032 mmol) in DMSO (2 mL) was added KOH (210 mg, 3.23 mmol) followed by iodomethane (0.2 mL, 3.23 mmol) at room temperature and stirred for 1 h. Water was added to the reaction mixture, extracted with EtOAc, the organic layer was washed with brine, dried over Na₂SO₄ and concentrated in vacuo. Purification by ISCO flash chromatography (12 g, 30% EtOAc in hexanes) gave a white solid of 2-chloro-5-isopropyl-5-methoxy-7,8-dihydro-6H-quinoline AK (80 mg, 0.333 mmol, 82% yield). Purity ≥95% by LCMS (210, 254 nm); tR=1.74 m/z=240.04 [M+H]⁺

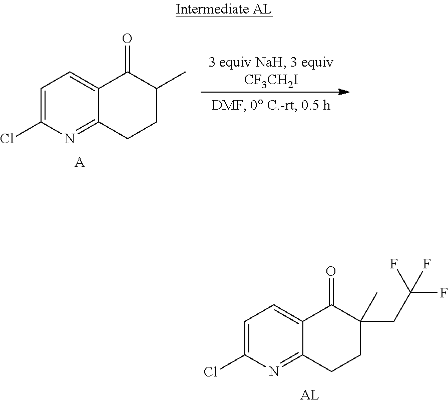

To 2-chloro-6-methyl-7,8-dihydro-6H-quinolin-5-one (120 mg, 0.61 mmol) in DMF (2.5 mL) was added sodium hydride (73 mg, 1.84 mmol) at 0° C. and stirred for 2 min. After addition of 1,1,1-Trifluoro-2-iodoethane (0.18 mL, 1.84 mmol) at 0° C., the reaction mixture was stirred at room temperature for 0.5 h. The reaction was quenched with water, extracted with EtOAc, the organic layer was dried over Na₂SO₄ and concentrated in vacuo. Purification by ISCO flash chromatography (12 g, 5% EtOAc in hexanes) gave a yellow oil of 2-chloro-6-methyl-6-(2,2,2-trifluoroethyl)-7,8-dihydroquinolin-5-one AL (25 mg, 0.090 mmol, 15% yield). Purity ≥95% by LCMS (210, 254 nm); tR=1.44 m/z=278.09 [M+H]⁺

### Example 1: Compound Syntheses

### Compound 1: 2-(3,5-difluoro-4-hydroxyphenyl)-7,8-dihydroquinolin-5(6H)-one

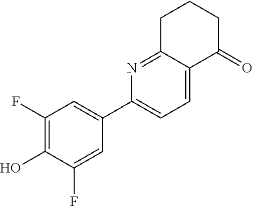 [SMILES: O=C1CCCc2nc(-c3cc(F)c(O)c(F)c3)ccc21]

Suzuki reaction: A mixture of 3,5-difluoro-4-hydroxyphenylboronic acid (101. mg, 0.58 mmol), 2-Chloro-7,8-dihydroquinolin-5 (6h)-one (105.48 mg, 0.58 mmol) and 2M aq. Potassium Carbonate (0.87 mL, 1.74 mmol) in Dioxane (3.2 mL) was degassed for 10 mins before adding XPhos palladacycle G2 (22.85 mg, 0.03 mmol). The reaction mixture was stirred at 90° C. for 2 h. After completion the mixture was diluted with EtOAc, poured into the separating funnel containing 1M HCl, organic layer was separated, dried over Na₂SO₄ and concentrated in vacuo. Purification by ISCO flash chromatography (12 g, 30% EtOAc in hexanes) gave a light yellow powder of 2-(3,5-difluoro-4-hydroxy-phenyl)-7,8-dihydro-6H-quinolin-5-one (50 mg 0.1817 mmol, 31% yield). Purity ≥95% by LCMS (210, 254 nm); tR=1.80 min, m/z=276.07 [M+H]⁺. ¹H NMR (400 MHZ, MeOD) δ 8.28 (d, J=8.3 Hz, 1H), 7.83-7.68 (m, 3H), 3.18 (t, J=6.4 Hz, 2H), 2.71 (t, J=6.4 Hz, 2H), 2.21 (p, J=6.4 Hz, 2H), ¹⁹F NMR (376 MHz, MeOD) δ −135.20.

### Compound 2—2-(3-fluoro-4-hydroxyphenyl)-7,8-dihydroquinolin-5(6H)-one

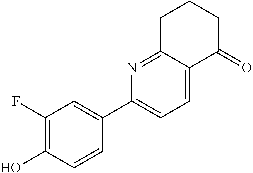 [SMILES: O=C1CCCc2nc(-c3ccc(O)c(F)c3)ccc21]

The title compound (45 mg, 0.121 mmol, 29% yield) was prepared following the procedures described in Example 1 substituting (3-fluoro-4-hydroxyphenyl)boronic acid in the Suzuki reaction step. Purity ≥95% by LCMS (210, 254 nm); tR=1.031 min, m/z=279.0923 [M+Na]⁺

### Compound 3—2-hydroxy-5-(5-oxo-5,6,7,8-tetrahydroquinolin-2-yl)benzonitrile

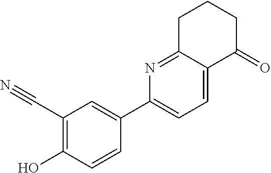 [SMILES: N#Cc1cc(-c2ccc3c(n2)CCCC3=O)ccc1O]

The title compound (51 mg, 0.19 mmol, 45% yield) was prepared following the procedures described in Example 1 substituting (3-cyano-4-hydroxyphenyl)boronic acid in the Suzuki reaction step. Purity ≥95% by LCMS (210, 254 nm); tR=1.737 min, m/z=265.0948 [M+H]⁺

### Compound 4—5-(6,6-dimethyl-5-oxo-5,6,7,8-tetrahydroquinolin-2-yl)-2-hydroxybenzonitrile

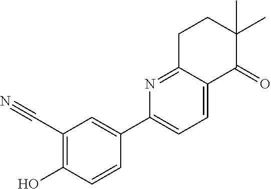 [SMILES: CC1(C)CCc2nc(-c3ccc(O)c(C#N)c3)ccc2C1=O]

The title compound (20 mg, 0.094 mmol, 27% yield) was prepared following the procedures described in Example 1 substituting (3-cyano-4-hydroxyphenyl)boronic acid and substituting 2-chloro-6,6-dimethyl-7,8-dihydroquinolin-5(6H)-one in the Suzuki reaction step. Purity ≥95% by LCMS (210, 254 nm); tR=1.10 m/z=293.12 [M+H]⁺. ¹H NMR (400 MHZ, MeOD) δ 8.38-8.31 (m, 2H), 8.23 (dd, J=8.8, 2.3 Hz, 1H), 7.84 (d, J=8.8 Hz, 1H), 7.09 (d, J=8.8 Hz, 1H), 3.23 (t, J=6.4 Hz, 2H), 2.08 (t, J=6.4 Hz, 2H), 1.23 (s, 6H). ¹⁹F NMR (376 MHZ, MeOD) δ −77.45.

### Compound 5—2-(3,5-difluoro-4-hydroxyphenyl)-6,6-dimethyl-7,8-dihydroquinolin-5(6H)-one

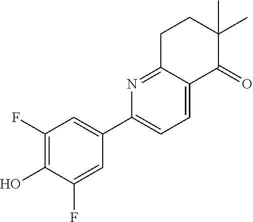 [SMILES: CC1(C)CCc2nc(-c3cc(F)c(O)c(F)c3)ccc2C1=O]

The title compound (100 mg, 0.33 mmol, 53% yield) was prepared following the procedures described in Example 1 substituting 2-chloro-6,6-dimethyl-7,8-dihydroquinolin-5(6H)-one in the Suzuki reaction step. Purity ≥95% by LCMS (210, 254 nm); tR=1.41 m/z=304.23 [M+H]⁺. ¹H NMR (400 MHZ, MeOD) δ 8.30 (d, J=8.3 Hz, 1H), 7.80 (d, J=8.3 Hz, 1H), 7.78-7.70 (m, 2H), 3.21 (t, J=6.4 Hz, 2H), 2.08 (t, J=6.4 Hz, 2H), 1.23 (s, 6H). ¹⁹F NMR (376 MHz, MeOD) δ −135.23.

### Compound 6—2-(3-fluoro-4-hydroxyphenyl)-6,6-dimethyl-7,8-dihydroquinolin-5(6H)-one

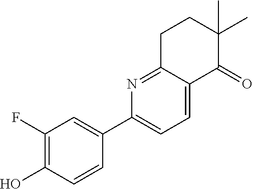 [SMILES: CC1(C)CCc2nc(-c3ccc(O)c(F)c3)ccc2C1=O]

The title compound (20 mg, 0.07 mmol, 22% yield) was prepared following the procedures described in Example 1 substituting (3-fluoro-4-hydroxyphenyl)boronic acid and 2-chloro-6,6-dimethyl-7,8-dihydroquinolin-5(6H)-one in the Suzuki reaction step. Purity ≥95% by LCMS (210, 254 nm); tR=0.90 m/z=286.12 [M+H]⁺. ¹H NMR (400 MHz, MeOD) δ 8.29 (d, J=8.3 Hz, 1H), 7.87 (dd, J=12.7, 2.4 Hz, 1H), 7.77 (m, 2H), 7.02 (t, J=8.8 Hz, 1H), 3.20 (t, J=6.5 Hz, 2H), 2.07 (t, J=6.5 Hz, 2H), 1.23 (s, 6H). ¹⁹F NMR (376 MHz, MeOD) δ −138.91.

### Compound 7—2-(3,5-difluoro-4-hydroxyphenyl)-6-methyl-7,8-dihydroquinolin-5(6H)-one

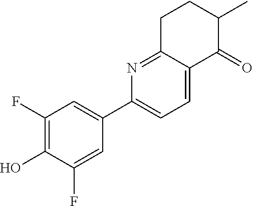 [SMILES: CC1CCc2nc(-c3cc(F)c(O)c(F)c3)ccc2C1=O]

The title compound (53 mg, 0.18 mmol, 59% yield) was prepared following the procedures described in Example 1 substituting 2-chloro-6-methyl-7,8-dihydroquinolin-5(6H)-one, obtained following intermediate A synthesis, in the Suzuki reaction step. Purity ≥95% by LCMS (210, 254 nm); tR=1.20 m/z=290.09 [M+H]⁺. H NMR (400 MHZ, MeOD) δ 8.29 (d, J=8.3 Hz, 1H), 7.82-7.68 (m, 3H), 3.27-3.17 (m, 2H), 2.70 (ddd, J=12.1, 6.8, 4.9 Hz, 1H), 2.33-2.27 (m, 1H), 1.99-1.84 (m, 1H), 1.27 (d, J=6.7 Hz, 3H). ¹⁹F NMR (376 MHz, MeOD) δ −135.23.

### Compound 8—2-(3,5-difluoro-4-hydroxyphenyl)-6-ethyl-7,8-dihydroquinolin-5(6H)-one

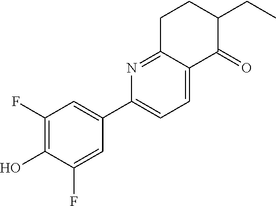 [SMILES: CCC1CCc2nc(-c3cc(F)c(O)c(F)c3)ccc2C1=O]

The title compound (10 mg, 0.033 mmol, 16% yield) was prepared following the procedures described in Example 1 substituting 2-chloro-6-ethyl-7,8-dihydroquinolin-5(6H)-one, obtained following intermediate A synthesis with ethyl iodide, in the Suzuki reaction step. Purity ≥95% by LCMS (210, 254 nm); tR=1.51 m/z=304.05 [M+H]⁺. ¹H NMR (400 MHZ, MeOD) δ 8.28 (d, J=8.3 Hz, 1H), 7.78 (d, J=8.3 Hz, 1H), 7.73 (dd, J=8.1, 1.9 Hz, 2H), 3.29-3.10 (m, 2H), 2.52 (dq, J=11.5, 5.5 Hz, 1H), 2.33 (dq, J=14.1, 4.8 Hz, 1H), 1.96 (tt, J=13.9, 5.0 Hz, 2H), 1.59 (dp, J=14.1, 7.5 Hz, 1H), 1.03 (t, J=7.5 Hz, 3H). ¹⁹F NMR (376 MHz, MeOD) δ −135.23

### Compound 9—6-benzyl-2-(3,5-difluoro-4-hydroxyphenyl)-7,8-dihydroquinolin-5(6H)-one

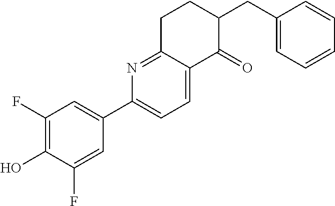 [SMILES: O=C1c2ccc(-c3cc(F)c(O)c(F)c3)nc2CCC1Cc1ccccc1]

The title compound (91 mg, 0.25 mmol, 45% yield) was prepared following the procedures described in Example 1 substituting 6-benzyl-2-chloro-7,8-dihydroquinolin-5(6H)-one, obtained following intermediate A synthesis with benzyl bromide, in the Suzuki reaction step. Purity ≥95% by LCMS (210, 254 nm); tR=1.86 m/z=366.26 [M+H]⁺. ¹H NMR (400 MHZ, MeOD) δ 8.30 (d, J=8.3 Hz, 1H), 7.82 (d, J=8.3 Hz, 1H), 7.77-7.67 (m, 2H), 7.33-7.28 (m, 4H), 7.22 (dp, J=8.7, 4.4, 3.8 Hz, 1H), 3.62 (dd, J=13.6, 5.2 Hz, 1H), 3.46-3.42 (m, 1H), 2.94 (dd, J=13.6, 9.6 Hz, 1H), 2.82 (ddd, J=17.3, 9.0, 4.5 Hz, 1H), 2.58 (ddd, J=17.3, 8.3, 4.7 Hz, 1H), 2.18 (ddt, J=13.8, 9.1, 4.4 Hz, 1H), 1.93 (p, J=7.7, 7.1 Hz, 1H). ¹⁹F NMR (376 MHz, MeOD) δ −135.23.

### Compound 10—2-(3,5-difluoro-4-hydroxyphenyl)-6-phenyl-7,8-dihydroquinolin-5(6H)-one

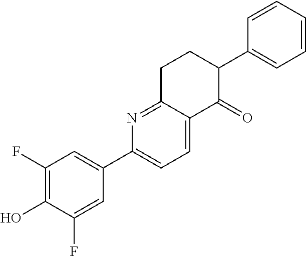 [SMILES: O=C1c2ccc(-c3cc(F)c(O)c(F)c3)nc2CCC1c1ccccc1]

The title compound (23 mg, 0.065 mmol, 26% yield) was prepared following the procedures described in Example 1 substituting 2-chloro-6-phenyl-7,8-dihydroquinolin-5(6H)-one, obtained following intermediate AE synthesis, in the Suzuki reaction step. Purity ≥95% by LCMS (210, 254 nm); tR=1.62 m/z=352.11 [M+H]⁺. ¹H NMR (400 MHZ, MeOD) δ 8.34 (d, J=8.4 Hz, 1H), 7.83 (d, J=8.4 Hz, 1H), 7.77 (dd, J=8.1, 2.0 Hz, 2H), 7.36-7.33 (m, 2H), 7.30-7.17 (m, 3H), 3.96 (dd, J=11.5, 4.9 Hz, 1H), 3.39-3.24 (m, 2H), 2.48 (dtd, J=18.1, 9.8, 8.7, 4.6 Hz, 2H). ¹⁹F NMR (376 MHz, MeOD) δ −135.19.

### Additional Compounds

The compounds shown in Table 1 were synthesized with methods identical or analogous to those described above. The Synthetic Example indicated in Table 1 refers to the compound identified above and corresponding synthetic method described therein. The requisite starting materials were commercially available (CA), described above, described in the literature, or readily synthesized by one skilled in the art of organic synthesis. The mass spectrometry data were obtained using TOF LC-MS Methods 1-3 as described above. LC-MS [M+H] means the 10 protonated mass of the free base of the compound.

TABLE 1
Additional Compounds and Characterization Data

| | | LC-MS | Retention | General | Intermediate |
|---|---|---|---|---|---|
| No. | Compound | [M + H] | time | Procedure | Example |
| 11 | 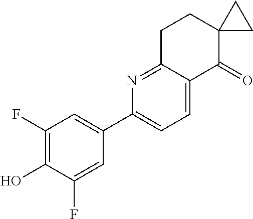 [SMILES: O=C1c2ccc(-c3cc(F)c(O)c(F)c3)nc2CCC12CC2] | 302.09 | 1.14 | 1 | G |
| 12 | 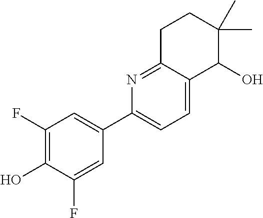 [SMILES: CC1(C)CCc2nc(-c3cc(F)c(O)c(F)c3)ccc2C1O] | 306.12 | 1.43 | 1 | B and D |
| 13 | 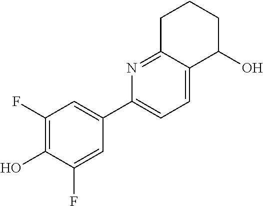 [SMILES: Oc1c(F)cc(-c2ccc3c(n2)CCCC3O)cc1F] | 278.00 | 1.23 | 1 | D |
| 14 | 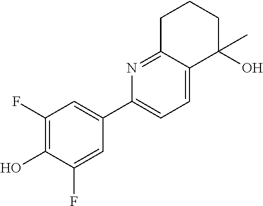 [SMILES: CC1(O)CCCc2nc(-c3cc(F)c(O)c(F)c3)ccc21] | 292.11 | 1.31 | 1 | AJ |
| 15 | 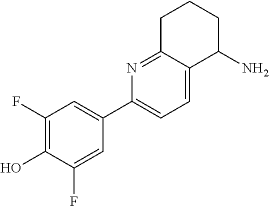 [SMILES: NC1CCCc2nc(-c3cc(F)c(O)c(F)c3)ccc21] | 277.13 | 1.18 | 1 | CA |
| 16 | 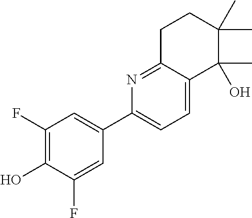 [SMILES: CC1(C)CCc2nc(-c3cc(F)c(O)c(F)c3)ccc2C1(C)O] | 320.17 | 1.47 | 1 | B and AJ |
| 17 | 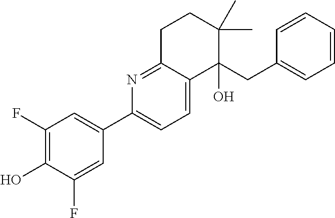 [SMILES: CC1(C)CCc2nc(-c3cc(F)c(O)c(F)c3)ccc2C1(O)Cc1ccccc1] | 396.18 | 1.85 | 1 | B and AJ |
| 18 | 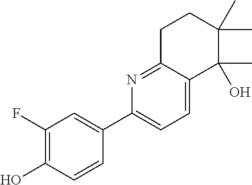 [SMILES: CC1(C)CCc2nc(-c3ccc(O)c(F)c3)ccc2C1(C)O] | 302.14 | 1.40 | 1 | B and AJ |
| 19 | 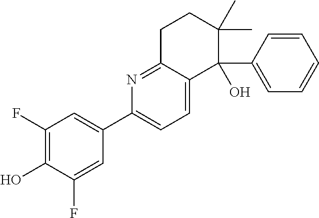 [SMILES: CC1(C)CCc2nc(-c3cc(F)c(O)c(F)c3)ccc2C1(O)c1ccccc1] | 382.17 | 1.85 | 1 | B and AJ |
| 20 | 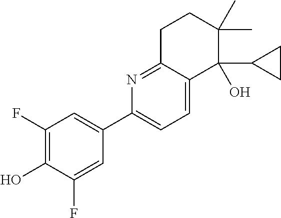 [SMILES: CC1(C)CCc2nc(-c3cc(F)c(O)c(F)c3)ccc2C1(O)C1CC1] | 346.17 | 1.61 | 1 | B and AJ |
| 21 | 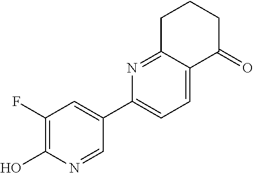 [SMILES: O=C1CCCc2nc(-c3cnc(O)c(F)c3)ccc21] | 259.08 | 1.528 | 1 | CA |
| 22 | 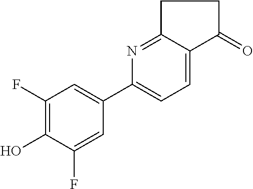 [SMILES: O=C1CCc2nc(-c3cc(F)c(O)c(F)c3)ccc21] | 262.07 | 1.75 | 1 | CA |
| 23 | 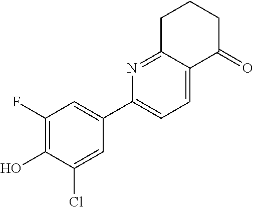 [SMILES: O=C1CCCc2nc(-c3cc(F)c(O)c(Cl)c3)ccc21] | 292.05 | 2.018 | 1 | CA |
| 24 | 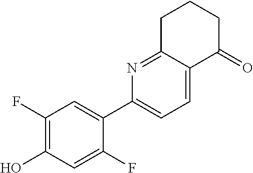 [SMILES: O=C1CCCc2nc(-c3cc(F)c(O)cc3F)ccc21] | 276.08 | 1.833 | 1 | CA |
| 25 | 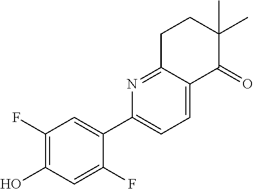 [SMILES: CC1(C)CCc2nc(-c3cc(F)c(O)cc3F)ccc2C1=O] | 304.11 | 2.146 | 1 | B |
| 26 | 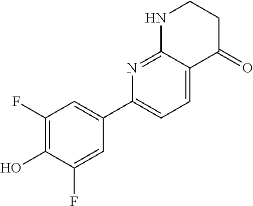 [SMILES: O=C1CCNc2nc(-c3cc(F)c(O)c(F)c3)ccc21] | 277.07 | 1.56 | 1 | CA |
| 27 | 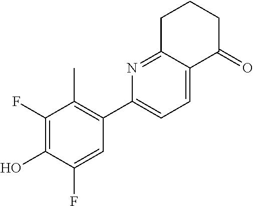 [SMILES: Cc1c(-c2ccc3c(n2)CCCC3=O)cc(F)c(O)c1F] | 290.1 | 1.73 | 1 | Q |
| 28 | 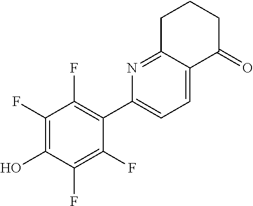 [SMILES: O=C1CCCc2nc(-c3c(F)c(F)c(O)c(F)c3F)ccc21] | 312.06 | 1.93 | 1 | P |
| 29 | 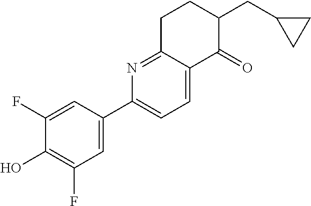 [SMILES: O=C1c2ccc(-c3cc(F)c(O)c(F)c3)nc2CCC1CC1CC1] | 330.12 | 1.74 | 1 | A |
| 30 | 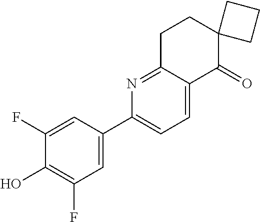 [SMILES: O=C1c2ccc(-c3cc(F)c(O)c(F)c3)nc2CCC12CCC2] | 316.12 | 2.29 | 1 | A |
| 31 | 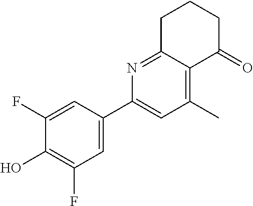 [SMILES: Cc1cc(-c2cc(F)c(O)c(F)c2)nc2c1C(=O)CCC2] | 290.09 | 1.81 | 3 | L |
| 32 | 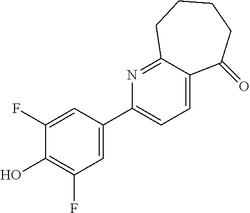 [SMILES: O=C1CCCCc2nc(-c3cc(F)c(O)c(F)c3)ccc21] | 290.09 | 1.84 | 1 | I |
| 33 | 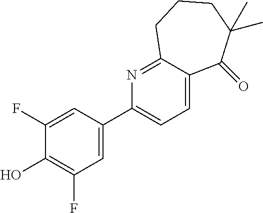 [SMILES: CC1(C)CCCc2nc(-c3cc(F)c(O)c(F)c3)ccc2C1=O] | 318.07 | 1.53 | 1 | J |
| 34 | 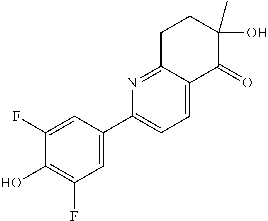 [SMILES: CC1(O)CCc2nc(-c3cc(F)c(O)c(F)c3)ccc2C1=O] | 306.09 | 1.73 | 1 | U |
| 35 | 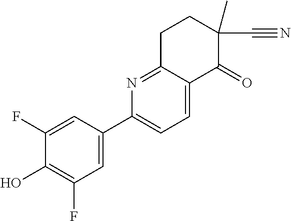 [SMILES: CC1(C#N)CCc2nc(-c3cc(F)c(O)c(F)c3)ccc2C1=O] | 315.09 | 1.10 | 2 | X |
| 36 | 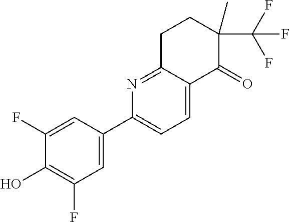 [SMILES: CC1(C(F)(F)F)CCc2nc(-c3cc(F)c(O)c(F)c3)ccc2C1=O] | 358.08 | 1.64 | 2 | AA |
| 37 | 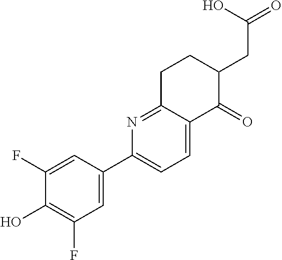 [SMILES: O=C(O)CC1CCc2nc(-c3cc(F)c(O)c(F)c3)ccc2C1=O] | 334.16 | 1.68 | 1 | A |
| 38 | 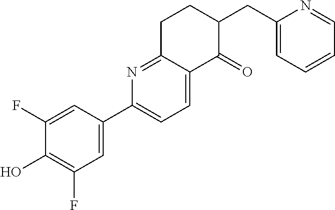 [SMILES: O=C1c2ccc(-c3cc(F)c(O)c(F)c3)nc2CCC1Cc1ccccn1] | 367.13 | 1.53 | 1 | A |
| 39 | 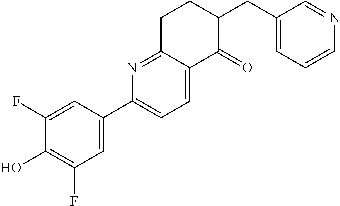 [SMILES: O=C1c2ccc(-c3cc(F)c(O)c(F)c3)nc2CCC1Cc1cccnc1] | 367.12 | 1.57 | 1 | A |
| 40 | 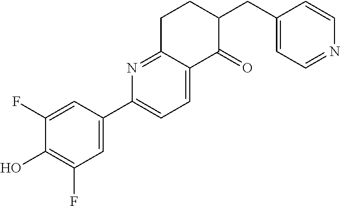 [SMILES: O=C1c2ccc(-c3cc(F)c(O)c(F)c3)nc2CCC1Cc1ccncc1] | 367.12 | 1.56 | 1 | A |
| 41 | 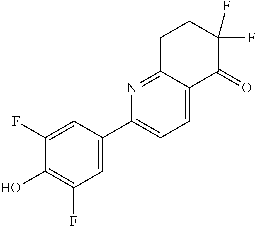 [SMILES: O=C1c2ccc(-c3cc(F)c(O)c(F)c3)nc2CCC1(F)F] | 312.06 | 1.28 | 1 | R |
| 42 | 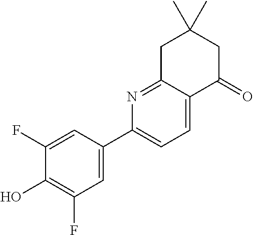 [SMILES: CC1(C)CC(=O)c2ccc(-c3cc(F)c(O)c(F)c3)nc2C1] | 304.11 | 2.13 | 1 | CA |
| 43 | 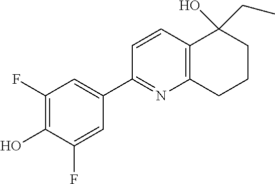 [SMILES: CCC1(O)CCCc2nc(-c3cc(F)c(O)c(F)c3)ccc21] | 306.01 | 1.42 | 1 | AJ |
| 44 | 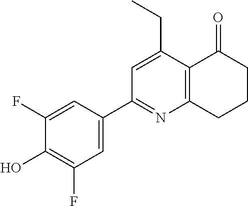 [SMILES: CCc1cc(-c2cc(F)c(O)c(F)c2)nc2c1C(=O)CCC2] | 304.20 | 2.00 | 3 | L |
| 45 | 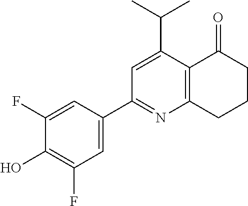 [SMILES: CC(C)c1cc(-c2cc(F)c(O)c(F)c2)nc2c1C(=O)CCC2] | 318.13 | 2.11 | 3 | L |
| 46 | 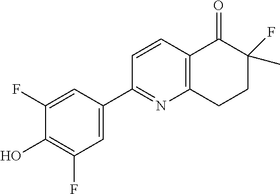 [SMILES: CC1(F)CCc2nc(-c3cc(F)c(O)c(F)c3)ccc2C1=O] | 308.08 | 1.17 | 2 | W |
| 47 | 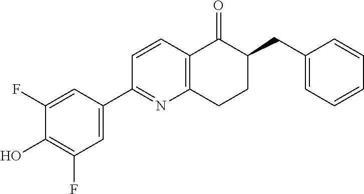 [SMILES: O=C1c2ccc(-c3cc(F)c(O)c(F)c3)nc2CC[C@@H]1Cc1ccccc1] | 366.13 | 2.37 | 1 | A |
| 48 | 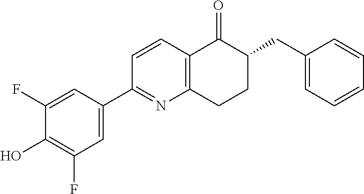 [SMILES: O=C1c2ccc(-c3cc(F)c(O)c(F)c3)nc2CC[C@H]1Cc1ccccc1] | 366.13 | 2.37 | 1 | A |
| 49 | 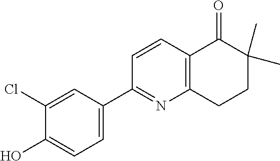 [SMILES: CC1(C)CCc2nc(-c3ccc(O)c(Cl)c3)ccc2C1=O] | 302.09 | 2.23 | 1 | B |
| 50 | 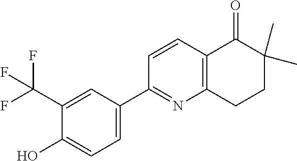 [SMILES: CC1(C)CCc2nc(-c3ccc(O)c(C(F)(F)F)c3)ccc2C1=O] | 336.12 | 2.31 | 1 | B |
| 51 | 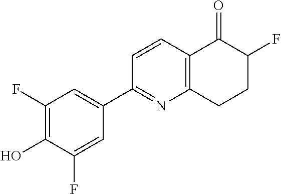 [SMILES: O=C1c2ccc(-c3cc(F)c(O)c(F)c3)nc2CCC1F] | 293.98 | 0.94 | 2 | AB |
| 52 | 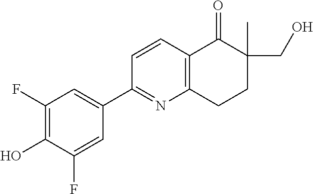 [SMILES: CC1(CO)CCc2nc(-c3cc(F)c(O)c(F)c3)ccc2C1=O] | 320.19 | 1.79 | 1 | S |
| 53 | 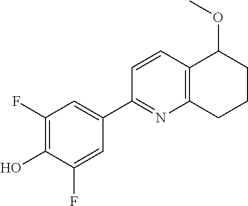 [SMILES: COC1CCCc2nc(-c3cc(F)c(O)c(F)c3)ccc21] | 292.19 | 1.62 | 1 | E |
| 54 | 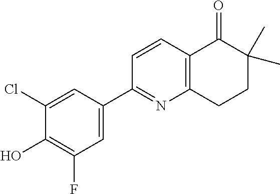 [SMILES: CC1(C)CCc2nc(-c3cc(F)c(O)c(Cl)c3)ccc2C1=O] | 320.11 | 2.33 | 1 | B |
| 55 | 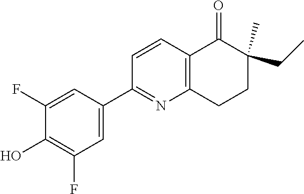 [SMILES: CC[C@@]1(C)CCc2nc(-c3cc(F)c(O)c(F)c3)ccc2C1=O] | 318.13 | 2.36 | 1 | AI-1 |
| 56 | 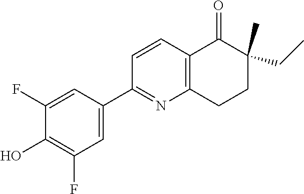 [SMILES: CC[C@]1(C)CCc2nc(-c3cc(F)c(O)c(F)c3)ccc2C1=O] | 318.13 | 2.37 | 1 | AI-2 |
| 57 | 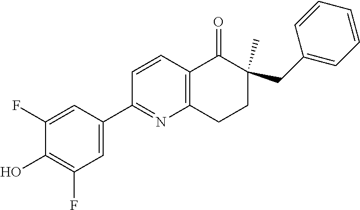 [SMILES: C[C@]1(Cc2ccccc2)CCc2nc(-c3cc(F)c(O)c(F)c3)ccc2C1=O] | 380.15 | 2.51 | 1 | AH-1 |
| 58 | 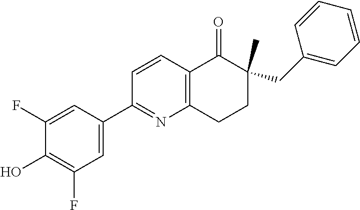 [SMILES: C[C@@]1(Cc2ccccc2)CCc2nc(-c3cc(F)c(O)c(F)c3)ccc2C1=O] | 380.15 | 2.49 | 1 | AH-2 |
| 59 | 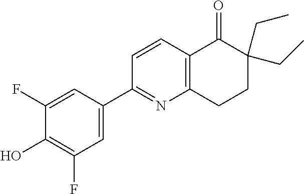 [SMILES: CCC1(CC)CCc2nc(-c3cc(F)c(O)c(F)c3)ccc2C1=O] | 332.15 | 2.49 | 1 | AG |
| 60 | 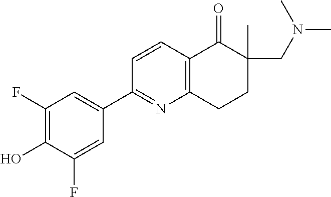 [SMILES: CN(C)CC1(C)CCc2nc(-c3cc(F)c(O)c(F)c3)ccc2C1=O] | 347.15 | 1.50 | 2 | Z |
| 61 | 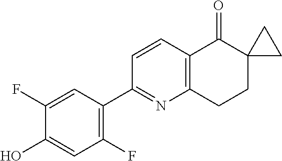 [SMILES: O=C1c2ccc(-c3cc(F)c(O)cc3F)nc2CCC12CC2] | 302.09 | 1.24 | 1 | G |
| 62 | 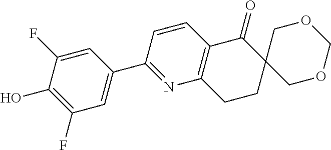 [SMILES: O=C1c2ccc(-c3cc(F)c(O)c(F)c3)nc2CCC12COCOC2] | 348.10 | 0.91 | 1 | AF |
| 63 | 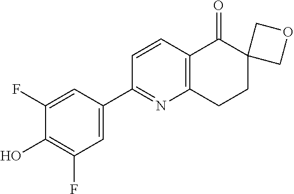 [SMILES: O=C1c2ccc(-c3cc(F)c(O)c(F)c3)nc2CCC12COC2] | 318.06 | 0.76 | 1 | F |
| 64 | 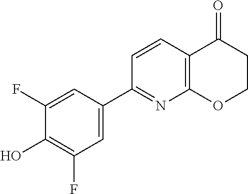 [SMILES: O=C1CCOc2nc(-c3cc(F)c(O)c(F)c3)ccc21] | 278.06 | 1.79 | 1 | CA |
| 65 | 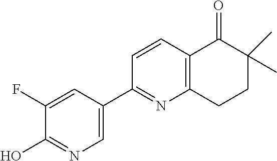 [SMILES: CC1(C)CCc2nc(-c3cnc(O)c(F)c3)ccc2C1=O] | 287.11 | 1.80 | 1 | B |
| 66 | 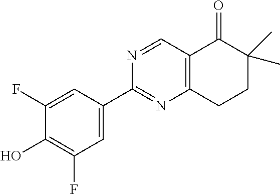 [SMILES: CC1(C)CCc2nc(-c3cc(F)c(O)c(F)c3)ncc2C1=O] | 305.18 | 1.40 | hand | AC |
| 67 | 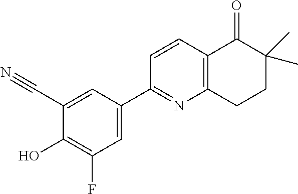 [SMILES: CC1(C)CCc2nc(-c3cc(F)c(O)c(C#N)c3)ccc2C1=O] | 311.11 | 1.45 | 1 | B |
| 68 | 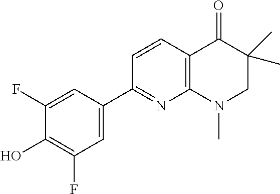 [SMILES: CN1CC(C)(C)C(=O)c2ccc(-c3cc(F)c(O)c(F)c3)nc21] | 319.12 | 1.60 | 1 | AD-2 |
| 69 | 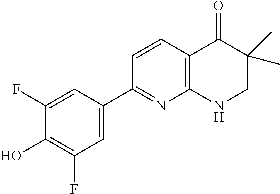 [SMILES: CC1(C)CNc2nc(-c3cc(F)c(O)c(F)c3)ccc2C1=O] | 305.11 | 1.89 | 1 | AD-1 |
| 70 | 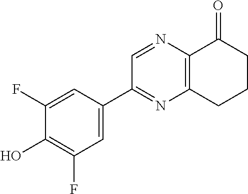 [SMILES: O=C1CCCc2nc(-c3cc(F)c(O)c(F)c3)cnc21] | 277.07 | 1.73 | 1 | K |
| 71 | 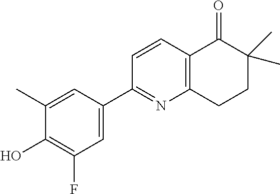 | 300.14 | 1.27 | 1 | B and N |
| 72 | 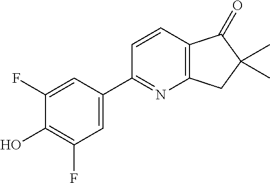 [SMILES: CC1(C)Cc2nc(-c3cc(F)c(O)c(F)c3)ccc2C1=O] | 290.10 | 1.19 | 1 | H |
| 73 | 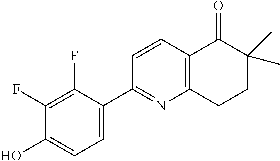 [SMILES: CC1(C)CCc2nc(-c3ccc(O)c(F)c3F)ccc2C1=O] | 304.11 | 1.29 | 1 | B |
| 74 | 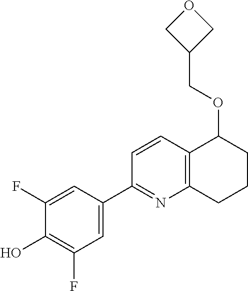 [SMILES: Oc1c(F)cc(-c2ccc3c(n2)CCCC3OCC2COC2)cc1F] | 348.08 | 1.50 | 1 | E |
| 75 | 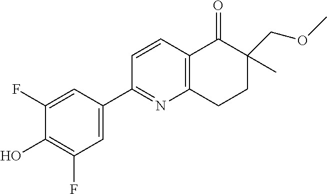 [SMILES: COCC1(C)CCc2nc(-c3cc(F)c(O)c(F)c3)ccc2C1=O] | 334.14 | 1.33 | 1 | T |
| 76 | 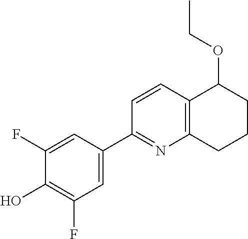 [SMILES: CCOC1CCCc2nc(-c3cc(F)c(O)c(F)c3)ccc21] | 306.00 | 1.67 | 1 | E |
| 77 | 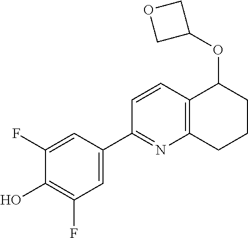 [SMILES: Oc1c(F)cc(-c2ccc3c(n2)CCCC3OC2COC2)cc1F] | 334.14 | 1.50 | 1 | E |
| 78 | 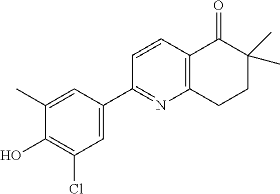 | 316.04 | 1.86 | 1 | B |
| 79 | 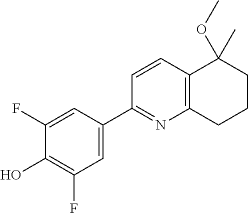 [SMILES: COC1(C)CCCc2nc(-c3cc(F)c(O)c(F)c3)ccc21] | 305.88 | 0.987 | 1 | AK |
| 80 | 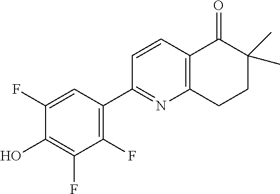 [SMILES: CC1(C)CCc2nc(-c3cc(F)c(O)c(F)c3F)ccc2C1=O] | 321.94 | 2.08 | 1 | B and O |
| 81 | 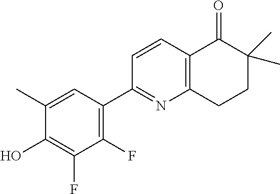 | 318.13 | 1.41 | 1 | B and M |
| 82 | 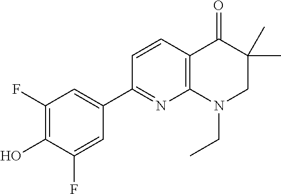 [SMILES: CCN1CC(C)(C)C(=O)c2ccc(-c3cc(F)c(O)c(F)c3)nc21] | 333.14 | 1.66 | 1 | AD-2 |
| 83 | 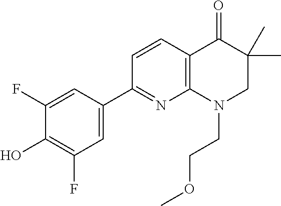 [SMILES: COCCN1CC(C)(C)C(=O)c2ccc(-c3cc(F)c(O)c(F)c3)nc21] | 363.15 | 1.46 | 1 | AD-2 |
| 84 | 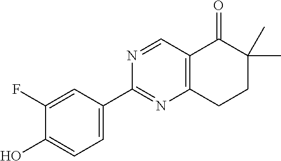 [SMILES: CC1(C)CCc2nc(-c3ccc(O)c(F)c3)ncc2C1=O] | 286.30 | 1.25 | 1 | AC |
| 85 | 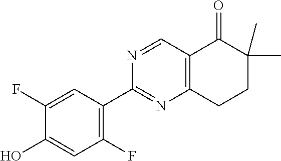 [SMILES: CC1(C)CCc2nc(-c3cc(F)c(O)cc3F)ncc2C1=O] | 305.11 | 1.14 | 1 | AC |
| 86 | 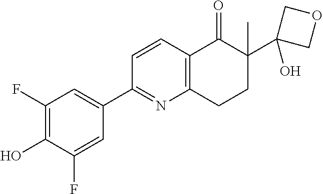 [SMILES: CC1(C2(O)COC2)CCc2nc(-c3cc(F)c(O)c(F)c3)ccc2C1=O] | 362.11 | 1.69 | 2 | Y |
| 87 | 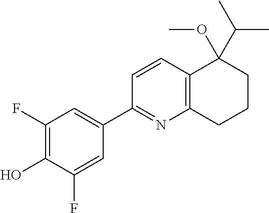 [SMILES: COC1(C(C)C)CCCc2nc(-c3cc(F)c(O)c(F)c3)ccc21] | 333.37 | 0.71 | 1 | AK |
| 88 | 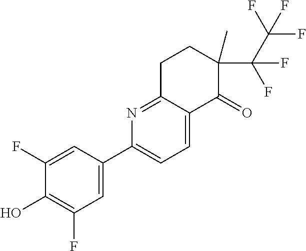 [SMILES: CC1(C(F)(F)C(F)(F)F)CCc2nc(-c3cc(F)c(O)c(F)c3)ccc2C1=O] | 408.10 | 1.79 | 2 | AA |
| 89 | 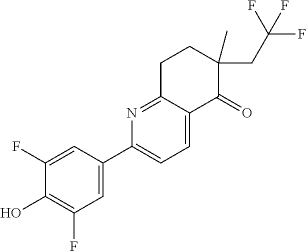 [SMILES: CC1(CC(F)(F)F)CCc2nc(-c3cc(F)c(O)c(F)c3)ccc2C1=O] | 372.15 | 1.55 | 1 | AL |
| 90 | 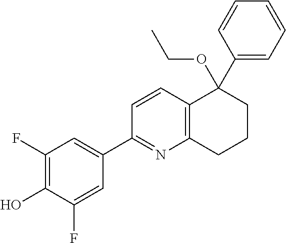 [SMILES: CCOC1(c2ccccc2)CCCc2nc(-c3cc(F)c(O)c(F)c3)ccc21] | 382.17 | 1.19 | 1 | AK |
| 91 | 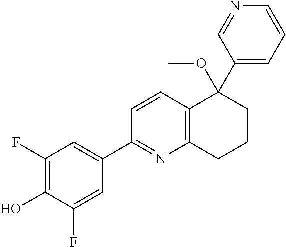 [SMILES: COC1(c2cccnc2)CCCc2nc(-c3cc(F)c(O)c(F)c3)ccc21] | 369.1805 | 1.43 | 1 | AK |
| 92 | 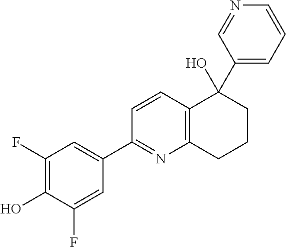 [SMILES: Oc1c(F)cc(-c2ccc3c(n2)CCCC3(O)c2cccnc2)cc1F] | 355.15 | 1.22 | 1 | AJ |
| 93 | 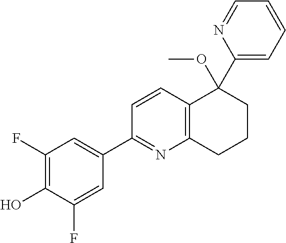 [SMILES: COC1(c2ccccn2)CCCc2nc(-c3cc(F)c(O)c(F)c3)ccc21] | 369.17 | 1.49 | 1 | AK |
| 94 | 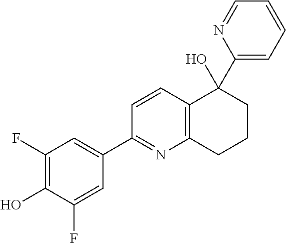 [SMILES: Oc1c(F)cc(-c2ccc3c(n2)CCCC3(O)c2ccccn2)cc1F] | 355.14 | 1.30 | 1 | AJ |
| 95 | 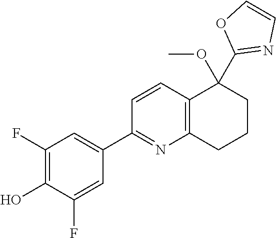 [SMILES: COC1(c2ncco2)CCCc2nc(-c3cc(F)c(O)c(F)c3)ccc21] | 359.12 | 1.64 | 1 | AK |

### Example 2: 3β-HSD1 Inhibition Assays

Compounds were demonstrated to enzymatically inhibit human 3β-HSD1 using a colorimetric NADH detection assay. Enzymatic turnover was quantified through NADH generation by purified, recombinant human 3β-HSD1 (expressed in Sf9 insect cells) in the presence of steroidal substrate trans-Dehydroandrosterone (DHEA) that was placed slightly above the experimentally determined Km and saturating amounts of co-factor Nicotinamide adenine dinucleotide (NAD⁺). The sequence of recombinant human 3β-HSD1 used is MHHHHHHENLYFQGTGWSCLVTGAGGFLGQRIIRLLVKEKELKEIRVLDKAFGPELREE FSKLQNKTKLTVLEGDILDEPFLKRACQDVSVIIHTACIIDVFGVTHRESIMNVNVKGTQL LLEACVQASVPVFIYTSSIEVAGPNSYKEIIQNGHEEEPLENTWPAPYPHSKKLAEKAVLA ANGWNLKNGGTLYTCALRPMYTYGEGSRFLSASINEALNNNGILSSVGKFSTVNPVYVG NVAWAHILALRALQDPKKAPSIRGQFYYISDDTPHQSYDNLNYTLSKEFGLRLDSRWSF PLSLMYWIGFLLEIVSFLLRPIYTYRPPFNRHIVTLSNSVFTFSYKKAQRDLAYKPLYSWE EAKQKTVEWVGSLVDRHKETLKSKTQ (SEQ ID NO: 3). DHEA and NAD+ were enzymatically converted to androstenedione and NADH, respectively. Final NADH concentrations were spectroscopically quantified using a commercially available Amplite™ Colorimetric NADH Assay Kit which utilizes an NADH probe with an absorbance wavelength of 460 nm. Enzymatic assays with a final volume of 25 μL were ran in a 384-well, Greiner Bio-One UV-STAR® assay plate in 50 mM TRIS pH 8.0 assay buffer. Compound IC₅₀ values were experimentally determined in duplicate using a 10-point concentration response curve (CRC) with a top concentration of 50 μM and a bottom concentration of 25 pM. Compound stocks at 10 mM in DMSO were serial diluted 5-fold in DMSO to generate an ECHO® compatible source plate. 125 nL of DMSO, Trilostane, or serial diluted compounds were then acoustically transferred into an empty UV-STAR® assay plate to give a final assay concentration of 0.5% v/v DMSO. The stamp volume and dilution scheme can be adjusted to accommodate compound potency. The assay can tolerate up to a 5% v/v DMSO final assay concentration. 15 μL of 1 μg purified 3β-HSD1 and 50 μM DHEA (final assay volume concentration) was dispensed per well into the stamped plate, centrifuged for 1 minute at 1000 RPM, covered, and allowed to incubate for 15 minutes at room temp. Enzymatic reactions were initiated upon the addition of 10 μL of NAD⁺ diluted in assay buffer to give a final assay volume concentration of 2 mM. The assay plate was centrifuged for 1 minute at 1000 RPM, covered, and incubated at room temperature for 1 hour. 25 μL Amplite™ Colorimetric NADH Assay Kit detection solution (prepared as recommended) was added to all reaction wells, the plate centrifuged for 1 minute at 1000 RPM, covered, and incubated for 15 minutes. Final absorbance values (λ=460 nm) are spectroscopically determined on BioTek® Cytation5 or Synergy4 plate readers. The background signal was determined by averaging 32 fully inhibited reactions (Trilostane treated) and subtracted across the plate. From the background subtracted values, the non-inhibited enzyme signal (DMSO treated) was averaged from 32 control reactions. The percent inhibition value was calculated by: % inhibition=(1−((background subtracted inhibited reaction)/(background subtracted non-inhibited average)))*100. The resulting percent inhibition was then plotted in duplicate against corresponding concentration values. The resulting CRCs were fit with a four parameter, variable slope log (inhibitor) vs response equation where Y=bottomᵥₐₗᵤₑ+ (topᵥₐₗᵤₑ−bottomᵥₐₗᵤₑ)/(1+10\hat{ }((log IC₅₀−X)*Hillslope)) to determine the IC₅₀ values for tested compounds.

TABLE 3
In vitro NADH inhibition of representative compounds

| Compound | IC₅₀ (nM) |
|---|---|
| 1 | 62 |
| 2 | 45 |
| 3 | 651 |
| 4 | 1861 |
| 5 | 71 |
| 6 | 51 |
| 7 | 64 |
| 8 | 105 |
| 9 | 37 |
| 10 | 79 |
| 11 | 68 |
| 12 | 2563 |
| 13 | 748 |
| 14 | 339 |
| 15 | 3442 |
| 16 | 376 |
| 17 | 227 |
| 18 | 504 |
| 19 | 314 |
| 20 | 331 |
| 21 | 6100 |
| 22 | 297 |
| 23 | 49 |
| 24 | 83 |
| 25 | 84 |
| 26 | 218 |
| 27 | 367 |
| 28 | 3000 |
| 29 | 130 |
| 30 | 303 |
| 31 | 991 |
| 32 | 90 |
| 33 | 292 |
| 34 | 446 |
| 35 | 92 |
| 36 | 59 |
| 37 | 2844 |
| 38 | 272 |
| 39 | 249 |
| 40 | 212 |
| 41 | 79 |
| 42 | 287 |
| 43 | 365 |
| 44 | 1281 |
| 45 | 8613 |
| 46 | 42 |
| 47 | 177 |
| 48 | 19 |
| 49 | 26 |
| 50 | 1498 |
| 51 | 149 |
| 52 | 222 |
| 53 | 204 |
| 54 | 107 |
| 55 | 89 |
| 56 | 110 |
| 57 | 78 |
| 58 | 55 |
| 59 | 71 |
| 60 | 1168 |
| 61 | 127 |
| 62 | 541 |
| 63 | 370 |
| 64 | 433 |
| 65 | >20000 |
| 66 | 414 |
| 67 | 1884 |
| 68 | 308 |
| 69 | 470 |
| 70 | 7638 |
| 71 | 67 |
| 72 | 221 |
| 73 | 122 |
| 74 | 441 |
| 75 | 338 |
| 76 | 389 |
| 77 | 1071 |
| 78 | 2168 |
| 79 | 38 |
| 80 | 152 |
| 81 | 326 |
| 82 | 326 |
| 83 | 556 |
| 84 | 288 |
| 85 | 1545 |
| 86 | 1422 |
| 87 | 45 |
| 88 | 78 |
| 89 | 188 |
| 90 | 52 |
| 91 | 25 |
| 92 | 68 |
| 93 | 131 |
| 94 | 238 |
| 95 | 100 |

### Example 3. Cellular Viability of Human Tumor Cell Lines

C4-2 cells were routinely maintained in RPMI+2 mM L-Glutamine+1× Anti-Anti+10% FBS. C4-2 cells were harvested by trypsinization for 90 seconds in 0.25% Trypsin-EDTA, counted with a Vi-Cell Blu cell counter (Beckman Coulter), and diluted into Complete Viability Media (RPMI+2 mM L-Glutamine+1× Anti-Anti+1% FBS+500 mM DHEA) at a density of 10,000 cells/mL. 12-point, two-fold dilution series of test compounds (starting at 10 mM) were prepared in 100% DMSO, and then 450 nL of the dilution series were stamped in triplicate into 384-well, white, opaque bottom, TC-treated CulturPlate™ (Perkin Elmer 6007680) using acoustic dispensing (Labcyte Echo 550). 50 μL/well of the diluted C4-2 cells (500 cells/well) were added directly to the plates containing dispensed compounds, yielding a final [DMSO] of 0.9% and a final top [compound] of 90 μM. The plates were centrifuged for 1 min at 500×g and then incubated for 6 days at 37° C., 5% CO₂. Cell density at the end of the assay was measured using 35 μL/well CellTiter-Glo® (Promega) per the manufacturer's recommendations, and total luminescence was measured on a Cytation 5 plate reader. Raw luminescence values were normalized from 0 (empty Viability Media+0.9% DMSO, no cells) to 100 (Cells+0.9% DMSO, maximum proliferation), triplicate values were averaged, and dose-response curves were fit and G150 values extracted using variable slope nonlinear regression in GraphPad Prism. Z′ values for all test plates was greater than 0.5, the positive control compound D4-Abiraterone was reproducible (GI₅₀=2.4±0.4 μM, n=7), and at least two biological replicates were completed for key compounds.

TABLE 4
Growth Inhibition (GI₅₀ in μM) for representative compounds
tested against C4-2 prostate cancer cells.

| Compound | GI₅₀ (μM) |
|---|---|
| 1 | 53 |
| 2 | 59 |
| 5 | 26 |
| 7 | 37 |
| 8 | 16 |
| 9 | 6 |
| 10 | 10 |
| 11 | 10 |
| 12 | 61 |
| 16 | 20 |
| 20 | 30 |

### Example 4. Cell Line Steroid Metabolism

Cells were seeded and incubated in 12-well plates with 0.15 million cells/well in RPMI-1640 medium with 10% FBS for ˜24 h at 37° C. Culture medium was then replaced with serum-free RPMI-1640 and a mixture of radioactive ([3H]-labeled) and non-radioactive androgens (final concentration, 25 nM; ˜600,000 cpm/well; PerkinElmer, Waltham, MA) plus inhibitor compounds at specified concentrations was added. Aliquots of medium were collected at 24 and 48 hour time points, then extracted with ethyl acetate:isooctane (1:1) and concentrated under nitrogen gas.

High-performance liquid chromatography (HPLC) analysis was performed on a Waters 1525 HPLC system (Waters Corp., Milford, MA). Dried samples were reconstituted in 50% methanol and injected into the HPLC. Steroids were separated on a Luna 150×4.6 mm, 3.0 μm particle size C18 reverse-phase column (Phenomenex, Torrance, CA) with a methanol/water gradient at 50° C. Column effluent was mixed with Liquiscint scintillation cocktail (National Diagnostics, Atlanta, GA) and analyzed using a β-RAM model 4 in-line radioactivity detector (LabLogic, Brandon, FL).

TABLE S
Androstenedione Synthesis Inhibition (IC₅₀ in nM) for
representative compounds tested in C4-2 prostate cancer cells

| Compound | IC₅₀ (nM) |
|---|---|
| 1 | 83 |
| 2 | 14 |
| 5 | 1.4 |
| 7 | 7.2 |
| 9 | 10 |
| 11 | 4.8 |
| 25 | 1 |
| 41 | 68 |
| 55 | 0.7 |
| 59 | 3.8 |

### Example 5. Activity of Compounds Using CRPC C4-2 Flank Tumor Model in Mice

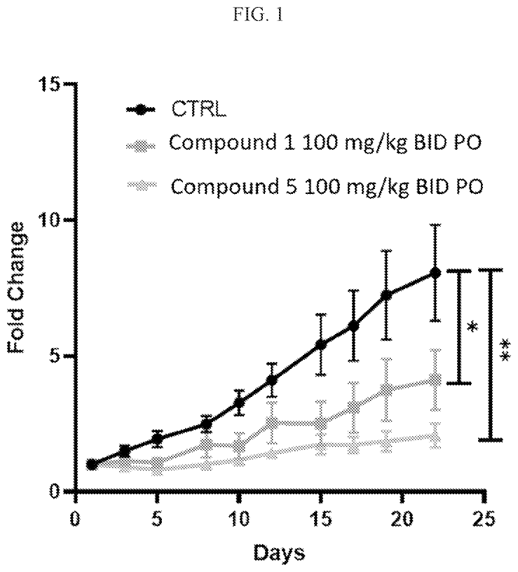

The mouse study was performed under a protocol approved by the Institutional Animal Care and Use Committee of the Cleveland Clinic Lerner Research Institute. All NOD scid gamma (NSG) male mice (6 to 8 weeks old) were purchased from the Jackson Laboratory. 10⁷ C4-2 cells were injected subcutaneously into mice with matrigel (total injection volume 100 μL). Once tumors reached 100 mm³ (length×width×height×0.52), mice were surgically orchiectomized and implanted with 5 mg 90-day sustained-release DHEA pellets (Innovative Research of America) to mimic human adrenal DHEA production in patients with CRPC, then the mice were arbitrarily (but not strictly randomized) assigned to vehicle (n=6), Compound 1 (n=6), or Compound 5 (n=6). The mice were given vehicle, Compound 1 (100 mg/kg in water with 20% Captisol), or Compound 5 (100 mg/kg in water with 20% Captisol) by oral gavage twice daily. The data are shown in FIG. 1, and demonstrate reduction in tumor volume for both test compounds compared to vehicle control.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I): 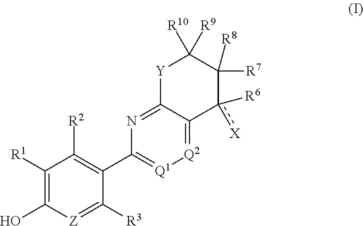
or a pharmaceutically acceptable salt thereof, wherein:
R¹ is halo, cyano, or C₁-C₄ haloalkyl;
Z is selected from CR⁴ and N;
R², R³, and R⁴ are independently selected from hydrogen, halo, and C₁-C₄ alkyl;
Q¹ is CH or N;
Q₂ is CR⁵ or N;
R⁵ is selected from hydrogen, C₁-C₄ alkyl, halo, and C₁-C₄ haloalkyl;
--- represents the presence or absence of a bond, wherein, when --- represents the presence of a bond, X is oxo and R⁶ is absent; and wherein, when --- represents the absence of a bond, X is selected from —OR^{a} and —NR^{b}R^{c}, and R⁶ is selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, and aryl-C₁-C₄-alkyl;
Y is selected from —CH₂—, —NR^{d}—, —O—, —S—, —CH₂CH₂—, —NHCH₂—, —OCH₂—, —SCH₂—, and a bond; and
R⁷, R⁸, R⁹, and R¹⁰ are each independently selected from hydrogen, C₁-C₆ alkyl, C₂-C₆ alkenyl, C₂-C₆ alkynyl, C₁-C₆ alkoxy, hydroxy, cyano, halo, C₃-C₆ cycloalkyl, monocyclic 3- to 6-membered heterocyclyl having one heteroatom selected from O and N, aryl, heteroaryl, C₁-C₄-alkoxy-C₁-C₆-alkyl, hydroxy-C₁-C₆-alkyl, halo-C₁-C₆-alkyl, carboxy-C₁-C₆-alkyl, amino-C₁-C₆-alkyl, C₃-C₆-cycloalkyl-C₁-C₄-alkyl, aryl-C₁-C₄-alkyl, and heteroaryl-C₁-C₄-alkyl, wherein R⁷ and R⁸, together with the carbon atom to which they are attached, are optionally taken together to form a 3- to 6-membered ring; and
R^{a}, R^{b}, R^{c}, and R^{d} are each independently selected from hydrogen, C₁-C₄ alkyl, C₁-C₄-alkoxy-C₁-C₄-alkyl, heterocyclyl, and heterocyclyl-C₁-C₄-alkyl;
wherein each cycloalkyl, heterocyclyl, aryl, and heteroaryl is independently unsubstituted or substituted with 1, 2, or 3 substituents independently selected from C₁-C₆ alkyl, C₁-C₆ alkoxy, hydroxy, cyano, halo, and C₃-C₆ cycloalkyl.

2. The compound of claim 1, or a pharmaceutically acceptable salt thereof, wherein R¹ is fluoro, chloro, cyano, or trifluoromethyl.

3. The compound of claim 1 or claim 2, or a pharmaceutically acceptable salt thereof, wherein R¹ is fluoro or cyano.

4. The compound of any one of claims 1-3, or a pharmaceutically acceptable salt thereof, wherein R² and R³ are each independently selected from hydrogen, fluoro, and methyl.

5. The compound of any one of claims 1-4, or a pharmaceutically acceptable salt thereof, wherein R² and R³ are each independently selected from hydrogen and fluoro.

6. The compound of any one of claims 1-5, or a pharmaceutically acceptable salt thereof, wherein R² and R³ are each hydrogen.

7. The compound of any one of claims 1-6, or a pharmaceutically acceptable salt thereof, wherein Z is CR⁴, and R⁴ is selected from hydrogen, halo, and methyl.

8. The compound of any one of claims 1-7, or a pharmaceutically acceptable salt thereof, wherein Z is CR⁴, and R⁴ is selected from hydrogen and halo.

9. The compound of any one of claims 1-6, or a pharmaceutically acceptable salt thereof, wherein Z is N.

10. The compound of any one of claims 1-9, or a pharmaceutically acceptable salt thereof, wherein Q¹ is CH.

11. The compound of any one of claims 1-9, or a pharmaceutically acceptable salt thereof, wherein Q¹ is N.

12. The compound of any one of claims 1-11, or a pharmaceutically acceptable salt thereof, wherein Q² is CR⁵, and R⁵ is selected from hydrogen and C₁-C₄-alkyl.

13. The compound of any one of claims 1-12, or a pharmaceutically acceptable salt thereof, wherein Q² is CR⁵, and R⁵ is hydrogen.

14. The compound of any one of claims 1-11, or a pharmaceutically acceptable salt thereof, wherein Q² is N.

15. The compound of any one of claims 1-14, or a pharmaceutically acceptable salt thereof, wherein --- represents the presence of a bond, X is oxo, and R⁶ is absent.

16. The compound of any one of claims 1-14, or a pharmaceutically acceptable salt thereof, wherein --- represents the absence of a bond, X is selected from —OR^{a} and —NR^{b}R^{c}, and R⁶ is selected from hydrogen, C₁-C₃ alkyl, C₃-C₄ cycloalkyl, phenyl, a monocyclic 5- or 6-membered heteroaryl having 1 or 2 heteroatoms independently selected from N, O, and S, and phenyl-C₁-C₂-alkyl, wherein R^{a} is selected from hydrogen, C₁-C₃ alkyl, heterocyclyl, and heterocyclyl-C₁-C₄-alkyl, and R^{b} and R^{c} are each hydrogen, wherein the heterocyclyl is a monocyclic 4- to 6-membered heterocyclyl having one heteroatom selected from O and N.

17. The compound of any one of claims 1-14, or a pharmaceutically acceptable salt thereof, wherein --- represents the absence of a bond; X is selected from —OH and —NH₂; and R⁶ is selected from hydrogen, C₁-C₈ alkyl, C₃-C₄ cycloalkyl, phenyl, pyridyl, oxazolyl, and phenyl-C₁-C₂-alkyl.

18. The compound of any one of claims 1-17, or a pharmaceutically acceptable salt thereof, wherein Y is selected from —CH₂—, —NR^{d}—, —O—, —CH₂CH₂—, and a bond, wherein R^{d} is selected from hydrogen, C₁-C₄ alkyl, and C₁-C₄-alkoxy-C₁-C₄-alkyl.

19. The compound of any one of claims 1-18, or a pharmaceutically acceptable salt thereof, wherein Y is selected from —CH₂—, —NH—, —CH₂CH₂—, and a bond.

20. The compound of any one of claims 1-19, or a pharmaceutically acceptable salt thereof, wherein R⁷ and R⁸ are each independently selected from hydrogen, C₁-C₃ alkyl, hydroxy, cyano, halo, C₃-C₆ cycloalkyl, a monocyclic 3- to 6-membered heterocyclyl having one heteroatom selected from O and N, aryl, C₁-C₂-alkoxy-C₁-C₃-alkyl, hydroxy-C₁-C₃-alkyl, halo-C₁-C₃-alkyl, carboxy-C₁-C₃-alkyl, amino-C₁-C₃-alkyl, C₃-C₄-cycloalkyl-C₁-C₂-alkyl, aryl-C₁-C₂-alkyl, and heteroaryl-C₁-C₂-alkyl, wherein the cycloalkyl and the heterocyclyl are each independently unsubstituted or substituted with one substituent selected from hydroxy, methyl, halo, and cyano.

21. The compound of any one of claims 1-20, or a pharmaceutically acceptable salt thereof, wherein R⁷ and R⁸ are each independently selected from hydrogen, C₁-C₃ alkyl, hydroxy, cyano, halo, C₃-C₆ cycloalkyl, aryl, halo-C₁-C₃-alkyl, carboxy-C₁-C₃-alkyl, C₃-C₄-cycloalkyl-C₁-C₂-alkyl, aryl-C₁-C₂-alkyl, and heteroaryl-C₁-C₂-alkyl.

22. The compound of any one of claims 1-19, or a pharmaceutically acceptable salt thereof, wherein R⁷ and R⁸, together with the carbon atom to which they are attached, are taken together to form a 3- to 6-membered cycloalkyl or a 3- to 6-membered monocyclic heterocyclyl having one or two oxygen atoms.

23. The compound of any one of claims 1-19, or a pharmaceutically acceptable salt thereof, wherein R⁷ and R⁸, together with the carbon atom to which they are attached, are taken together to form a 3- or 4-membered cycloalkyl.

24. The compound of any one of claims 1-23, or a pharmaceutically acceptable salt thereof, wherein R⁹ and R¹⁰ are each independently hydrogen or C₁-C₄ alkyl.

25. The compound of any one of claims 1-24, or a pharmaceutically acceptable salt thereof, wherein R⁹ and R¹⁰ are each independently hydrogen or methyl.

26. The compound of claim 1, selected from the group consisting of: 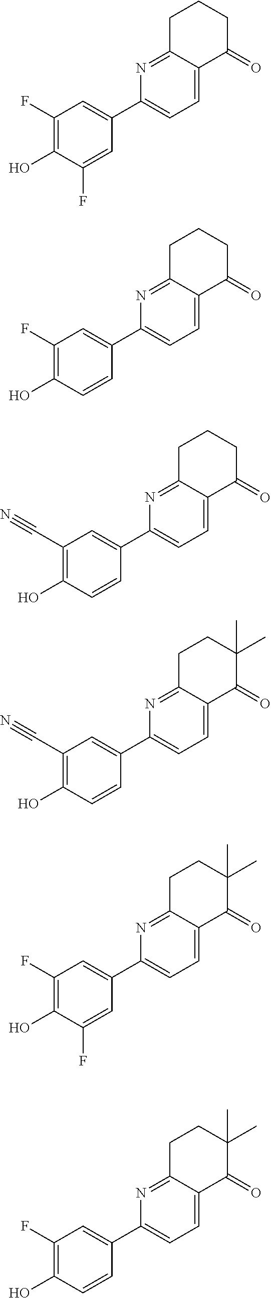 [SMILES: O=C1CCCc2nc(-c3cc(F)c(O)c(F)c3)ccc21 ; O=C1CCCc2nc(-c3ccc(O)c(F)c3)ccc21 ; N#Cc1cc(-c2ccc3c(n2)CCCC3=O)ccc1O ; CC1(C)CCc2nc(-c3ccc(O)c(C#N)c3)ccc2C1=O ; CC1(C)CCc2nc(-c3cc(F)c(O)c(F)c3)ccc2C1=O ; CC1(C)CCc2nc(-c3ccc(O)c(F)c3)ccc2C1=O] 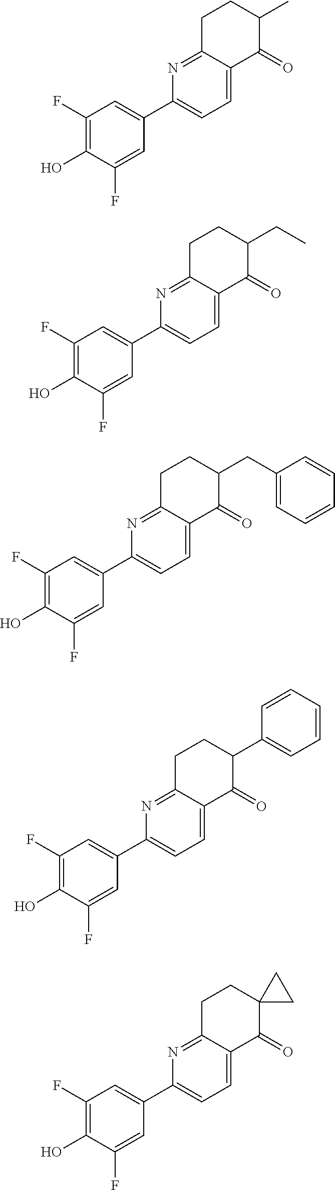 [SMILES: CC1CCc2nc(-c3cc(F)c(O)c(F)c3)ccc2C1=O ; CCC1CCc2nc(-c3cc(F)c(O)c(F)c3)ccc2C1=O ; O=C1c2ccc(-c3cc(F)c(O)c(F)c3)nc2CCC1Cc1ccccc1 ; O=C1c2ccc(-c3cc(F)c(O)c(F)c3)nc2CCC1c1ccccc1 ; O=C1c2ccc(-c3cc(F)c(O)c(F)c3)nc2CCC12CC2] 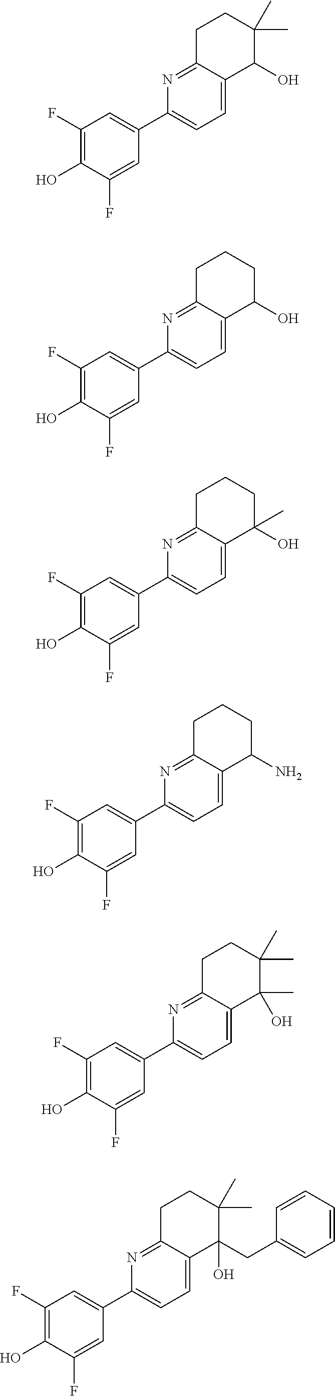 [SMILES: CC1(C)CCc2nc(-c3cc(F)c(O)c(F)c3)ccc2C1O ; Oc1c(F)cc(-c2ccc3c(n2)CCCC3O)cc1F ; CC1(O)CCCc2nc(-c3cc(F)c(O)c(F)c3)ccc21 ; NC1CCCc2nc(-c3cc(F)c(O)c(F)c3)ccc21 ; CC1(C)CCc2nc(-c3cc(F)c(O)c(F)c3)ccc2C1(C)O ; CC1(C)CCc2nc(-c3cc(F)c(O)c(F)c3)ccc2C1(O)Cc1ccccc1] 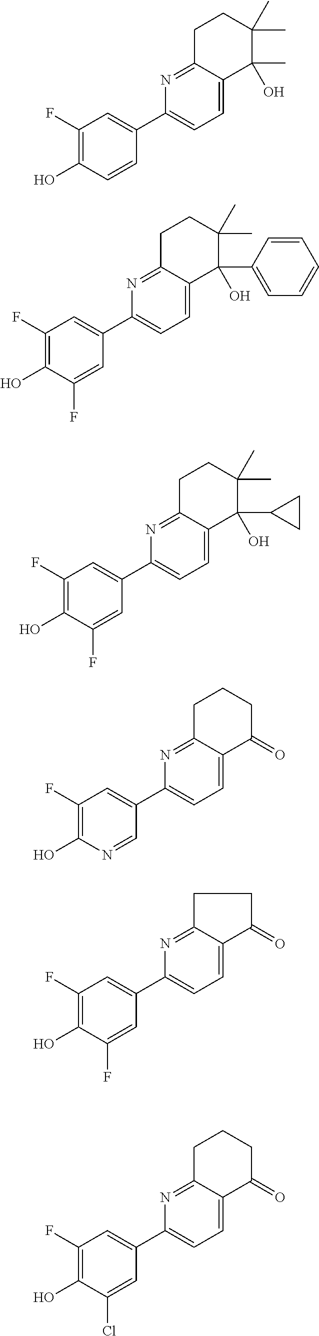 [SMILES: CC1(C)CCc2nc(-c3ccc(O)c(F)c3)ccc2C1(C)O ; CC1(C)CCc2nc(-c3cc(F)c(O)c(F)c3)ccc2C1(O)c1ccccc1 ; CC1(C)CCc2nc(-c3cc(F)c(O)c(F)c3)ccc2C1(O)C1CC1 ; O=C1CCCc2nc(-c3cnc(O)c(F)c3)ccc21 ; O=C1CCc2nc(-c3cc(F)c(O)c(F)c3)ccc21 ; O=C1CCCc2nc(-c3cc(F)c(O)c(Cl)c3)ccc21] 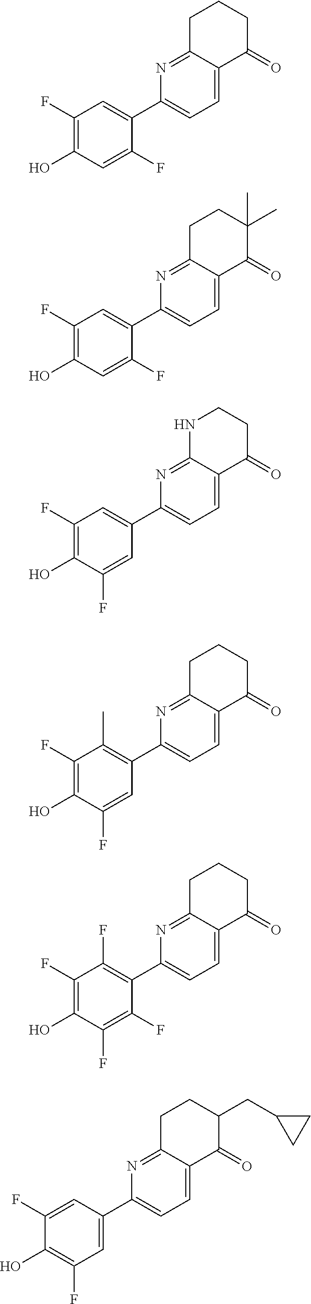 [SMILES: O=C1CCCc2nc(-c3cc(F)c(O)cc3F)ccc21 ; CC1(C)CCc2nc(-c3cc(F)c(O)cc3F)ccc2C1=O ; O=C1CCNc2nc(-c3cc(F)c(O)c(F)c3)ccc21 ; Cc1c(-c2ccc3c(n2)CCCC3=O)cc(F)c(O)c1F ; O=C1CCCc2nc(-c3c(F)c(F)c(O)c(F)c3F)ccc21 ; O=C1c2ccc(-c3cc(F)c(O)c(F)c3)nc2CCC1CC1CC1] 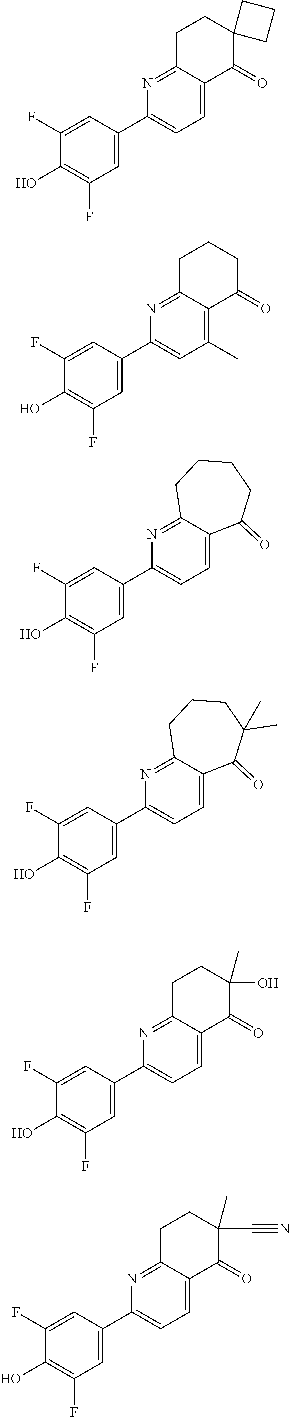 [SMILES: O=C1c2ccc(-c3cc(F)c(O)c(F)c3)nc2CCC12CCC2 ; Cc1cc(-c2cc(F)c(O)c(F)c2)nc2c1C(=O)CCC2 ; O=C1CCCCc2nc(-c3cc(F)c(O)c(F)c3)ccc21 ; CC1(C)CCCc2nc(-c3cc(F)c(O)c(F)c3)ccc2C1=O ; CC1(O)CCc2nc(-c3cc(F)c(O)c(F)c3)ccc2C1=O ; CC1(C#N)CCc2nc(-c3cc(F)c(O)c(F)c3)ccc2C1=O] 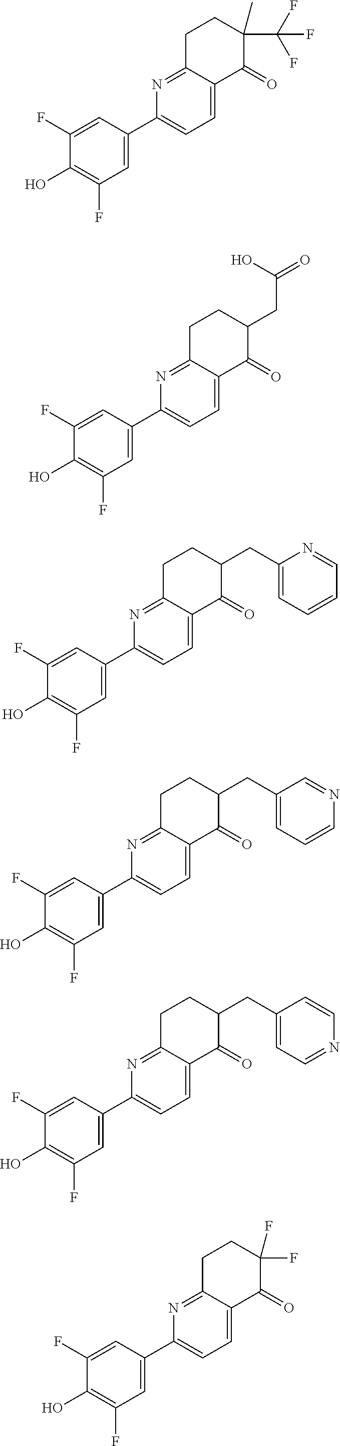 [SMILES: CC1(C(F)(F)F)CCc2nc(-c3cc(F)c(O)c(F)c3)ccc2C1=O ; O=C(O)CC1CCc2nc(-c3cc(F)c(O)c(F)c3)ccc2C1=O ; O=C1c2ccc(-c3cc(F)c(O)c(F)c3)nc2CCC1Cc1ccccn1 ; O=C1c2ccc(-c3cc(F)c(O)c(F)c3)nc2CCC1Cc1cccnc1 ; O=C1c2ccc(-c3cc(F)c(O)c(F)c3)nc2CCC1Cc1ccncc1 ; O=C1c2ccc(-c3cc(F)c(O)c(F)c3)nc2CCC1(F)F] 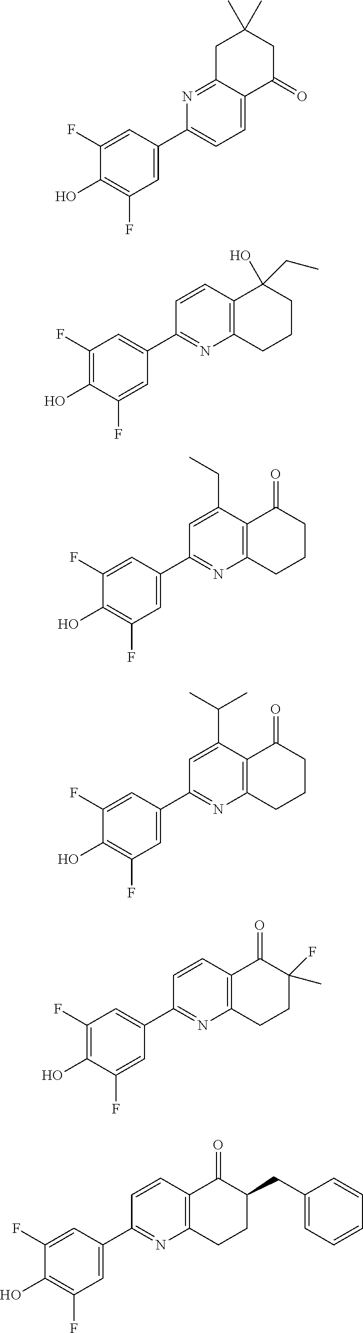 [SMILES: CC1(C)CC(=O)c2ccc(-c3cc(F)c(O)c(F)c3)nc2C1 ; CCC1(O)CCCc2nc(-c3cc(F)c(O)c(F)c3)ccc21 ; CCc1cc(-c2cc(F)c(O)c(F)c2)nc2c1C(=O)CCC2 ; CC(C)c1cc(-c2cc(F)c(O)c(F)c2)nc2c1C(=O)CCC2 ; CC1(F)CCc2nc(-c3cc(F)c(O)c(F)c3)ccc2C1=O ; O=C1c2ccc(-c3cc(F)c(O)c(F)c3)nc2CC[C@@H]1Cc1ccccc1] 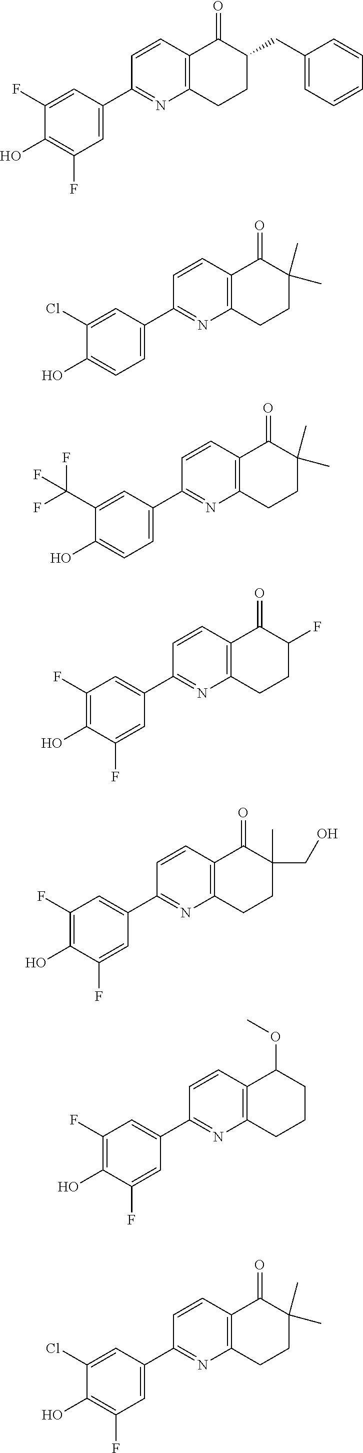 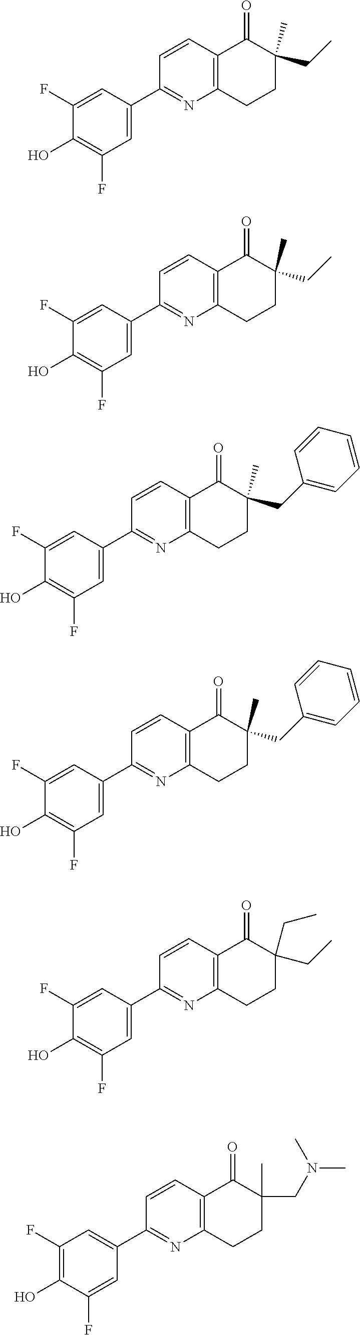 [SMILES: CC[C@@]1(C)CCc2nc(-c3cc(F)c(O)c(F)c3)ccc2C1=O ; CC[C@]1(C)CCc2nc(-c3cc(F)c(O)c(F)c3)ccc2C1=O ; C[C@]1(Cc2ccccc2)CCc2nc(-c3cc(F)c(O)c(F)c3)ccc2C1=O ; C[C@@]1(Cc2ccccc2)CCc2nc(-c3cc(F)c(O)c(F)c3)ccc2C1=O ; CCC1(CC)CCc2nc(-c3cc(F)c(O)c(F)c3)ccc2C1=O ; CN(C)CC1(C)CCc2nc(-c3cc(F)c(O)c(F)c3)ccc2C1=O] 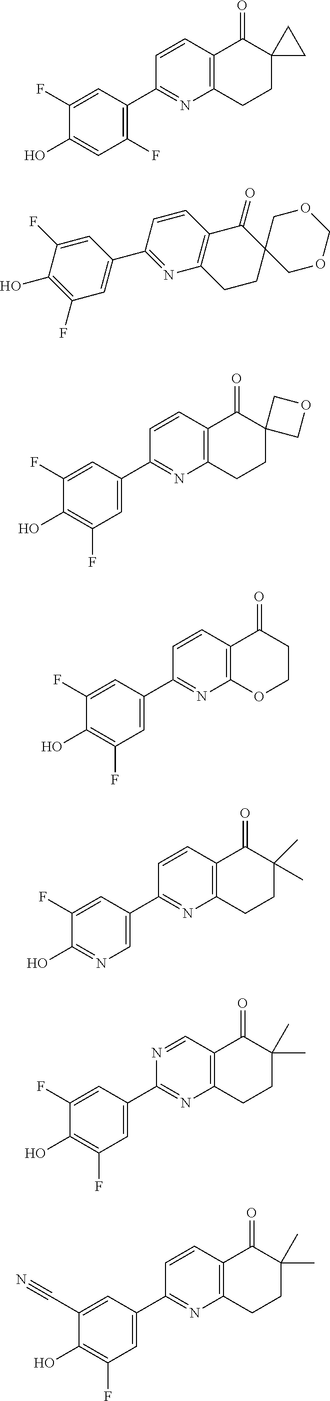 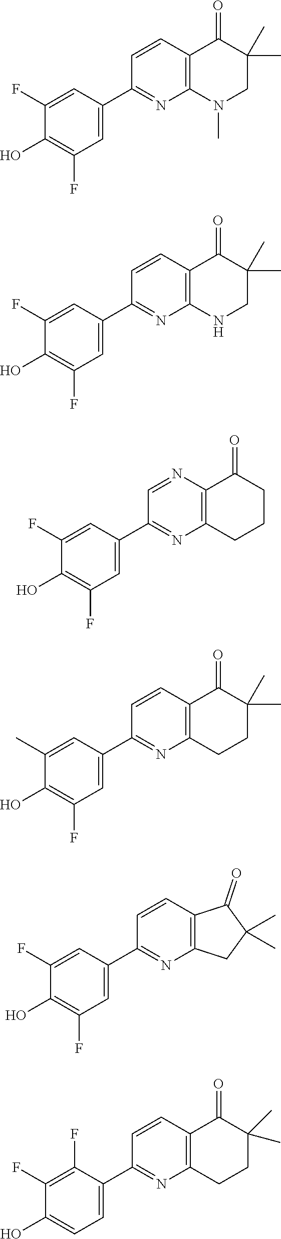 [SMILES: CN1CC(C)(C)C(=O)c2ccc(-c3cc(F)c(O)c(F)c3)nc21 ; CC1(C)CNc2nc(-c3cc(F)c(O)c(F)c3)ccc2C1=O ; O=C1CCCc2nc(-c3cc(F)c(O)c(F)c3)cnc21 ; Cc1cc(-c2ccc3c(n2)CCC(C)(C)C3=O)cc(F)c1O ; CC1(C)Cc2nc(-c3cc(F)c(O)c(F)c3)ccc2C1=O ; CC1(C)CCc2nc(-c3ccc(O)c(F)c3F)ccc2C1=O] 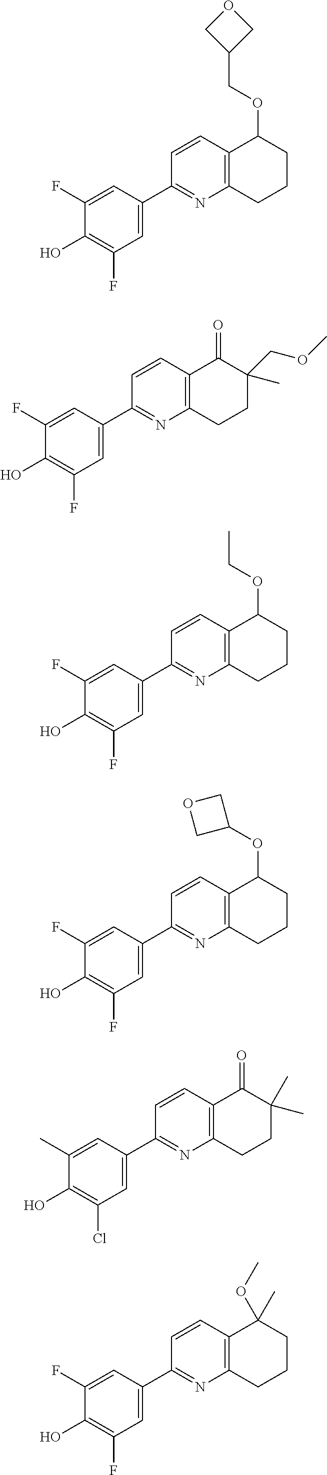 [SMILES: Oc1c(F)cc(-c2ccc3c(n2)CCCC3OCC2COC2)cc1F ; COCC1(C)CCc2nc(-c3cc(F)c(O)c(F)c3)ccc2C1=O ; CCOC1CCCc2nc(-c3cc(F)c(O)c(F)c3)ccc21 ; Oc1c(F)cc(-c2ccc3c(n2)CCCC3OC2COC2)cc1F ; Cc1cc(-c2ccc3c(n2)CCC(C)(C)C3=O)cc(Cl)c1O ; COC1(C)CCCc2nc(-c3cc(F)c(O)c(F)c3)ccc21] 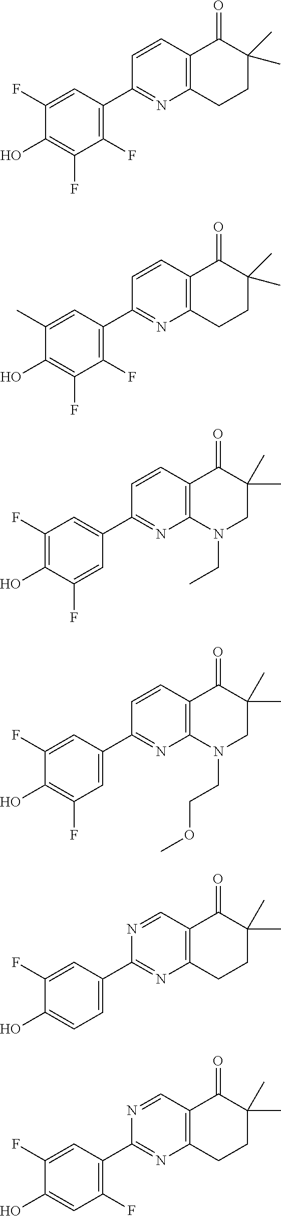 [SMILES: CC1(C)CCc2nc(-c3cc(F)c(O)c(F)c3F)ccc2C1=O ; Cc1cc(-c2ccc3c(n2)CCC(C)(C)C3=O)c(F)c(F)c1O ; CCN1CC(C)(C)C(=O)c2ccc(-c3cc(F)c(O)c(F)c3)nc21 ; COCCN1CC(C)(C)C(=O)c2ccc(-c3cc(F)c(O)c(F)c3)nc21 ; CC1(C)CCc2nc(-c3ccc(O)c(F)c3)ncc2C1=O ; CC1(C)CCc2nc(-c3cc(F)c(O)cc3F)ncc2C1=O] 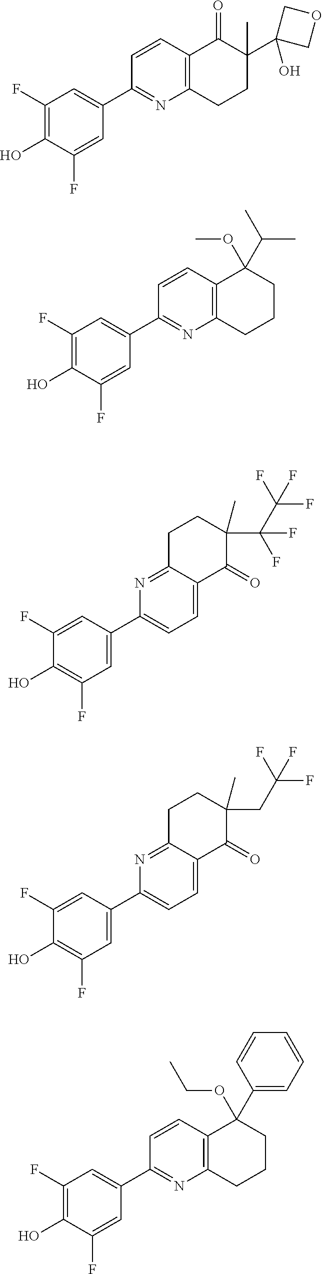 [SMILES: CC1(C2(O)COC2)CCc2nc(-c3cc(F)c(O)c(F)c3)ccc2C1=O ; COC1(C(C)C)CCCc2nc(-c3cc(F)c(O)c(F)c3)ccc21 ; CC1(C(F)(F)C(F)(F)F)CCc2nc(-c3cc(F)c(O)c(F)c3)ccc2C1=O ; CC1(CC(F)(F)F)CCc2nc(-c3cc(F)c(O)c(F)c3)ccc2C1=O ; CCOC1(c2ccccc2)CCCc2nc(-c3cc(F)c(O)c(F)c3)ccc21] 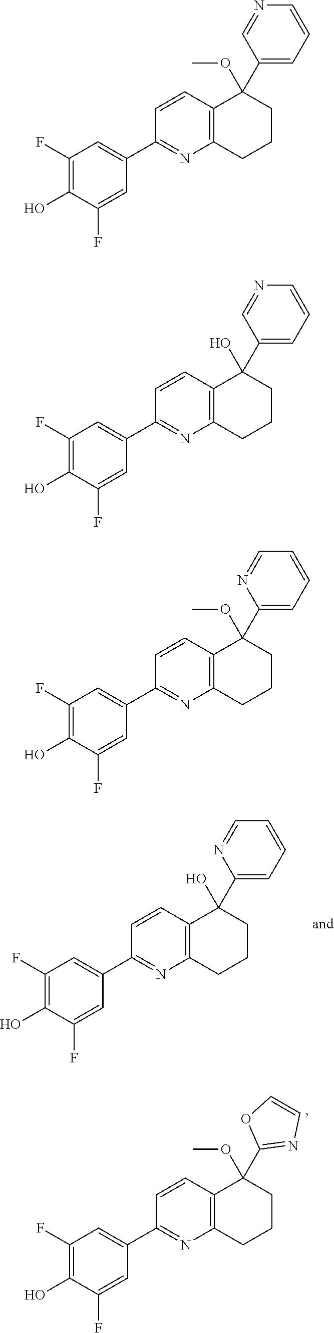 [SMILES: COC1(c2cccnc2)CCCc2nc(-c3cc(F)c(O)c(F)c3)ccc21 ; Oc1c(F)cc(-c2ccc3c(n2)CCCC3(O)c2cccnc2)cc1F ; COC1(c2ccccn2)CCCc2nc(-c3cc(F)c(O)c(F)c3)ccc21 ; Oc1c(F)cc(-c2ccc3c(n2)CCCC3(O)c2ccccn2)cc1F ; COC1(c2ncco2)CCCc2nc(-c3cc(F)c(O)c(F)c3)ccc21]
and pharmaceutically acceptable salts thereof.

27. A pharmaceutical composition comprising a compound of any one of claims 1-26, or a pharmaceutically acceptable salt thereof, and a pharmaceutically acceptable carrier.

28. A method of treating cancer in a subject in need thereof, comprising administering to the subject a therapeutically effective amount of a compound of any one of claims 1-26, or a pharmaceutically acceptable salt thereof.

29. The method of claim 28, wherein the cancer is prostate cancer, breast cancer, or endometrial cancer.

30. The method of claim 28, wherein the cancer is castration-resistant prostate cancer.

31. The method of any one of claims 28-30, further comprising treating the subject with one or more additional therapies.

32. The method of claim 31, wherein the one or more additional therapies are selected from surgery, chemotherapy, radiation therapy, hormone therapy, immunotherapy, cryotherapy, and thermotherapy, or any combination thereof.

33. A method of inhibiting cancer cell proliferation, comprising contacting cancer cells with a compound of any one of claims 1-26, or a pharmaceutically acceptable salt thereof, in an amount effective to inhibit the cancer cell proliferation.

34. The method of claim 33, wherein the cancer cells are prostate cancer cells, breast cancer cells, or endometrial cancer cells.

35. A method of inhibiting the activity of 3β-hydroxysteroid dehydrogenase in a sample, comprising contacting the sample with a compound of any one of claims 1-26, or a pharmaceutically acceptable salt thereof, in an amount effective to inhibit the activity of 3β-hydroxysteroid dehydrogenase.

36. A method of treating cancer in a subject in need thereof, the method comprising:
a) determining that a cytosine nucleotide is present at position 1245 of the HSD3β1 gene or a threonine is present at position 367 of the 3βHSD1 protein by assaying a biological sample from the subject, and
b) administering a therapeutically effective amount of the compound of any one of claims 1-26, or a pharmaceutically acceptable salt thereof, to the subject.

37. The method of claim 36, wherein the cancer is prostate cancer, breast cancer, or endometrial cancer.

38. The method of claim 36 or claim 37, wherein the biological sample comprises cancer cells.
